SEQUENCE LISTING (33 sequences)

SEQ ID NO: 1 - DNA, 20 bp, Artificial Sequence
  ARID1A forward
tcatgcccaaccttcgtatc

SEQ ID NO: 2 - DNA, 19 bp, Artificial Sequence
  ARID1A reverse
gatggctgctgggagtatg

SEQ ID NO: 3 - DNA, 20 bp, Artificial Sequence
  HDAC1 forward
aatgaagcctcaccgaatcc

SEQ ID NO: 4 - DNA, 21 bp, Artificial Sequence
  HDAC1 reverse
gctgtggtacttggtcatctc

SEQ ID NO: 5 - DNA, 24 bp, Artificial Sequence
  HDAC2 forward
catgacccataacttgctgttaaa

SEQ ID NO: 6 - DNA, 24 bp, Artificial Sequence
  HDAC2 reverse
atctggtcttattgaccgtagaaa

SEQ ID NO: 7 - DNA, 20 bp, Artificial Sequence
  HDAC3 forward
catagcctggtcctgcatta

SEQ ID NO: 8 - DNA, 22 bp, Artificial Sequence
  HDAC3 reverse
ctctctgcaggaagtcaatgta

SEQ ID NO: 9 - DNA, 20 bp, Artificial Sequence
  HDAC4 forward
cagaagcagcagatccagag

SEQ ID NO: 10 - DNA, 17 bp, Artificial Sequence
  HDAC4 reverse
tgcttcatggccagcat

SEQ ID NO: 11 - DNA, 18 bp, Artificial Sequence
  HDAC5 forward
aagagccatgcccagttc

SEQ ID NO: 12 - DNA, 17 bp, Artificial Sequence
  HDAC5 reverse
ctcctgctgcagttgct

SEQ ID NO: 13 - DNA, 22 bp, Artificial Sequence
  HDAC6 forward
tggtgttggatgagcagttaaa

SEQ ID NO: 14 - DNA, 18 bp, Artificial Sequence
  HDAC6 reverse
aggacacgcagcgatcta

SEQ ID NO: 15 - DNA, 21 bp, Artificial Sequence
  HDAC7 forward
gcttctccacaaggacaagag

SEQ ID NO: 16 - DNA, 22 bp, Artificial Sequence
  HDAC7 reverse
gttgggatggactgttctttct

SEQ ID NO: 17 - DNA, 22 bp, Artificial Sequence
  HDAC8 forward
gcattcagtttcacctccaaag

SEQ ID NO: 18 - DNA, 21 bp, Artificial Sequence
  HDAC8 reverse
tacactgtagtaccgtccctt

SEQ ID NO: 19 - DNA, 20 bp, Artificial Sequence
  HDAC9 forward
aggacagacctcaggatgat

SEQ ID NO: 20 - DNA, 21 bp, Artificial Sequence
  HDAC9 reverse
ctgcttctggatttgttgctg

SEQ ID NO: 21 - DNA, 21 bp, Artificial Sequence
  HDAC10 forward
gcacagcccagagtatgtatc

SEQ ID NO: 22 - DNA, 21 bp, Artificial Sequence
  HDAC10 reverse
gaagtagatggcgtcgaactg

SEQ ID NO: 23 - DNA, 21 bp, Artificial Sequence
  HDAC11 forward
gtctacaaccgccacatctac

SEQ ID NO: 24 - DNA, 24 bp, Artificial Sequence
  HDAC11 reverse
cttgatgttcctctccaccttatc

SEQ ID NO: 25 - DNA, 21 bp, Artificial Sequence
  p53 forward - ORF
ccagatgaagctcccagaatg

SEQ ID NO: 26 - DNA, 20 bp, Artificial Sequence
  p53 reverse - ORF
tgggaagggacagaagatga

SEQ ID NO: 27 - DNA, 23 bp, Artificial Sequence
  p53 forward - 3′ UTR
ggagtaggacataccagcttaga

SEQ ID NO: 28 - DNA, 23 bp, Artificial Sequence
  p53 reverse - 3′ UTR
cctacctagaatgtggctgattg

SEQ ID NO: 29 - DNA, 19 bp, Artificial Sequence
  B2M forward
ggcattcctgaagctgaca

SEQ ID NO: 30 - DNA, 23 bp, Artificial Sequence
  B2M reverse
cttcaatgtcggatggatgaaac

SEQ ID NO: 31 - DNA, 22 bp, Artificial Sequence
  HDAC6 promoter forward
actaagactccttccactcaga

SEQ ID NO: 32 - DNA, 21 bp, Artificial Sequence
  HDAC6 promoter reverse
actatggtggtacacaggaaa

SEQ ID NO: 33 - DNA, 23 bp, Artificial Sequence
  ARID1A gRNA sequence
cgggttgcccaggctgctggcgg

(19) United States
(12) Patent Application Publication
(10) Pub. No.: US 2019/0192521 A1
(43) Pub. Date: Jun. 27, 2019

(54) Methods of Treating Arid1A-Mutated Cancers With HDAC6 Inhibitors and EZH2 Inhibitors

(71) Applicant: The Wistar Institute of Anatomy and Biology, Philadelphia, PA (US)

(72) Inventors: Rugang Zhang, Elkins Park, PA (US); Benjamin Bitler, Elkins Park, PA (US)

(21) Appl. No.: 16/325,836

(22) PCT Filed: Jul. 24, 2017
(86) PCT No.: PCT/US2017/043521

§ 371 Date: Feb. 15, 2019

Related U.S. Application Data
(60) Provisional application No. 62/375,153, filed on Aug. 15, 2016.

(51) Int. Cl. A61K 31/519; A61K 31/422; A61K 31/167; A61K 31/66; A61K 31/4045; A61K 31/496; A61K 31/5377; A61K 31/444; A61K 31/4439; A61K 31/437; A61P 35/00

(52) U.S. Cl. CPC A61K 31/519; A61K 31/422; A61K 31/167; A61K 31/66; A61K 31/4045; A61P 35/00; A61K 31/5377; A61K 31/444; A61K 31/4439; A61K 31/437; A61K 31/496

(57) ABSTRACT

In some embodiments, therapeutic treatments for a disease such as a cancer are disclosed, including pharmaceutical compositions and methods of using pharmaceutical compositions for treating the cancer, wherein the cancer is an ARID1A-mutated cancer. In some embodiments, the therapeutic treatments disclosed include methods of treating ARID1A-mutated cancer in a subject comprising the step of administering a therapeutically effective dose of a histone deacetylase 6 (HDAC6) inhibitor to the subject, including a human subject. In some embodiments, the HDAC6 inhibitors are administered in conjunction with a therapeutically effective dose of an enhancer of zeste homolog 2 (EZH2) inhibitor.

### FIELD OF THE INVENTION

Therapeutic treatments of ARID1A-mutated cancers, such as ARID1A-mutated ovarian cancers, are disclosed.

### BACKGROUND OF THE INVENTION

Ovarian cancer is currently the eight most common cancer among women, but is the fifth leading cause of cancer-related death. SEER Cancer Statistics Factsheets: Ovary Cancer. National Cancer Institute, Bethesda, Md., 2013. Despite the recent advances in targeted therapy in different types of cancer, the mortality rate for patients with ovarian cancer has declined only slightly over the last 40 years. Cancer Facts and Figures 2016, American Cancer Society, Atlanta, 2016. Most ovarian cancer patients continue to be treated with chemotherapeutic agents, such as platinum-based antineoplastic drugs and taxanes, but the five-year overall survival rate remains less than 50%. Epigenetic regulation has been shown to play an important role in ovarian cancer and its subtypes, and the identification of both epigenetic categories of ovarian cancers and targeted treatments for these categories is critical for improvement of survival.

SWI/SNF (switch/sucrose non-fermentable) chromatin remodeling complexes regulate gene transcription by altering chromatin structure through hydrolysis of adenosine triphosphate (ATP), and are dysregulated in different types of cancer. Wilson and Roberts, Nat. Rev. Cancer 2011, 11, 481-92. Mutations in genes encoding subunits of the SWI/SNF complexes collectively occur in ˜20% of all human cancers. Kadoch et al. Nat. Genet. 2013, 45, 592-601. For example, saturation analysis of The Cancer Genome Atlas (TCGA) cancer mutational profile reveals that the ARID1A subunit of the SWI/SNF complex shows one of the highest mutation rates among epigenetic regulators. The AT-rich interactive domain-containing protein 1A (ARID1A) subunit of the SWI/SNF complex shows high mutation rates among epigenetic regulators, and is mutated in over 50% of ovarian clear cell carcinomas and 30% of ovarian endometrioid carcinomas. Lawrence et al. Nature 2014, 505, 495-501; Jones et al. Science 2010, 330, 228-231; Wiegand et al. N Engl. J. Med. 2010, 363, 1532-1543. ARID1A mutation is also a known genetic driver of ovarian cancer. Chandler et al. Nature Commun. 2015, 6, 6118; Guan et al. J. Natl. Cancer Inst. 2014, 106, 1-4; Zhai et al. J. Pathol. 2016, 238, 21-30. ARID1A and TP53 mutations are typically mutually exclusive in ovarian cancer. Guan et al. Cancer Research 2011, 71, 6718-6727. Because TP53 helps conserve genomic stability, ARID1A-mutated ovarian cancers often lack genomic instability. However, therapeutic approaches to harness the genetic characteristic of ARID1A-mutated cancers remain unavailable.

The histone deacetylase (HDAC) enzymes are a class of enzymes responsible for deacetylation of N-acetyl lysine residues on histones, affecting the ability of histones to wrap deoxyribonucleic acid and ultimately the regulation of DNA expression. Approximately eighteen HDACs are currently known, with diverse roles and functions, and of which many are under investigation as potential targets for cancer therapies. West and Johnstone, J. Clin. Invest. 2014, 124, 30-39; Mottamal et al. Molecules 2015, 20, 3898-3941. The known HDACs have been classified into four major classes based on homology, known as Classes I, II, III, and IV. Classes I, II, and IV are Zn²⁺-dependent metalloproteins, whereas Class III is a nicotinamide adenine dinucleotide (NAD⁺)-dependent enzyme family (also known as the sirtuin family). Within each class, the individual HDACs have unique functions and properties. For example, histone deacetylase 6 (HDAC6), which belongs to a subclass of class II (class IIb), is unique compared to other HDACs because of its cytoplasmic functionality and because it does not appear to directly deacetylate histones. Li et al. FEBS J. 2013, 280, 775-793. HDAC6 expression is increased in a number of cancer types, including ovarian cancer. Bazzaro et al. Clin. Cancer Res. 2008, 14, 7340-7347.

A large number of HDAC inhibitors have been developed and explored as potential cancer targets. Specific small molecule HDAC6 inhibitors have been developed only recently, and are being tested in human clinical trials for hematopoietic malignancies such as lymphoma and myeloma. Santo et al. Blood 2012, 119, 2579-2589; Mottamal et al. Molecules 2015, 20, 3898-3941. Pan-HDAC inhibitors capable of inhibiting HDAC6 as well as other HDAC family members, such as vorinostat, have also been developed and tested in the clinic. West and Johnstone, J. Clin. Invest. 2014, 124, 30-39. However, the inhibition of HDAC6 activity in ARID1A-mutant cancers has not been explored.

Polycomb-repressive complex 2 (PRC2) is a multiprotein complex that negatively regulates the expression of large numbers of genes by generating a silencing histone modification (H3K27me3) through its catalytic subunit enhancer of zeste homolog 2 (EZH2). Many of the genes regulated by PRC2 are involved in cancer progression, and dysregulation of PRC2 function is observed in many different types of cancer including EOC. Recent studies have shown that SWI/SNF and PRC2 complexes play an antagonistic role in tumorigenesis. Wilson et al. Cancer Cell 2010, 184, 316-328. EZH2 is highly expressed in many cancers, including breast cancer, prostate cancer, and lymphoma, and is frequently associated with tumor progression and poor outcomes. Furthermore, mutated forms of EZH2, including somatic heterozygous mutations of the Y641 and A677 residues of the catalytic SET domain, are observed in some cancers including diffuse large B-cell lymphoma (DLBCL) and follicular lymphoma. Morin et al. Nature 2011, 476, 298-303; Ryan et al. PLoS ONE 2011, 6, e28585; Morin et al. Nature Genet. 2010, 42, 181-185.

A number of selective small molecule EZH2 inhibitors have been identified and progressed into clinical development. Momparler and Côté, Expert Opin. Investig. Drugs 2015, 24, 1031-43; Melnick, Cancer Cell 2012, 22, 569-70. EZH2 inhibitors are currently being investigated for the treatment of cancers exhibiting overexpression of EZH2, including B cell lymphomas such as DLBCL, Germinal center B-cell DLBCL (GCB-DLBCL), and non-Hodgkin's lymphoma, follicular lymphoma, multiple myeloma, INI1-negative tumors, synovial sarcoma, breast cancer, prostate cancer, and other solid tumors. For example, the EZH2 inhibitor tazemetostat (EPZ-6438) has potent activity against EZH2-mutated non-Hodgkin's lymphoma. Knutson et al. Mol. Cancer. Ther. 2014, 13, 842-854.

The present invention provides the unexpected finding that HDAC6 inhibitors, alone or in combination with EZH2 inhibitors, may be used to effectively treat ARID1A-mutated cancers, including ARID1A-mutated ovarian cancers, ARID1A-mutated non-small-cell lung cancers, and ARID1A-mutated renal cancers.

### SUMMARY OF THE INVENTION

In an embodiment, the invention includes a method of treating a cancer in a human subject having a mutation in the ARID1A gene, comprising the step of administering a therapeutically effective dose of a HDAC6 inhibitor to the human subject in need thereof.

In an embodiment, the invention includes a method of treating a cancer in a human subject having a mutation in the ARID1A gene, comprising the step of administering a therapeutically effective dose of a HDAC6 inhibitor to the human subject in need thereof, wherein the cancer is selected from the group consisting of ovarian cancer, non-small-cell lung cancer, and renal cancer.

In an embodiment, the invention includes a method of treating a cancer in a human subject having a mutation in the ARID1A gene, comprising the step of administering a therapeutically effective dose of a HDAC6 inhibitor to the human subject in need thereof, wherein the cancer is ovarian cancer.

In an embodiment, the invention includes a method of treating a cancer in a human subject having a mutation in the ARID1A gene, comprising the step of administering a therapeutically effective dose of a HDAC6 inhibitor to the human subject in need thereof, wherein the cancer is epithelial ovarian cancer.

In an embodiment, the invention includes a method of treating a cancer in a human subject having a mutation in the ARID1A gene, comprising the step of administering a therapeutically effective dose of a HDAC6 inhibitor to the human subject in need thereof, wherein the cancer is ovarian clear cell carcinoma.

In an embodiment, the invention includes a method of treating a cancer in a human subject having a mutation in the ARID1A gene, comprising the step of administering a therapeutically effective dose of a HDAC6 inhibitor to the human subject in need thereof, further comprising detecting the presence of the mutation in the ARID1A gene in a tissue sample isolated from the human subject.

In an embodiment, the invention includes a method of treating a cancer in a human subject having a mutation in the ARID1A gene, comprising the step of administering a therapeutically effective dose of a HDAC6 inhibitor to the human subject in need thereof, further comprising detecting the presence of the mutation in the ARID1A gene in a tissue sample isolated from the human subject, wherein the human subject in need thereof has been selected from human subjects suffering from a cancer who do not have a mutation in the ARID1A gene.

In an embodiment, the invention includes a method of treating a cancer in a human subject having a mutation in the ARID1A gene, comprising the step of administering a therapeutically effective dose of a HDAC6 inhibitor to the human subject in need thereof, wherein the HDAC6 inhibitor is selected from the group consisting of rocilinostat:

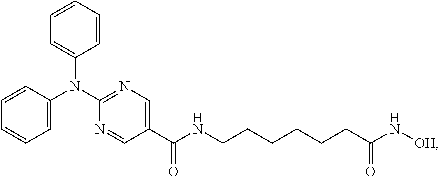 [SMILES: O=C(CCCCCCNC(=O)c1cnc(N(c2ccccc2)c2ccccc2)nc1)NO]

ACY-241:

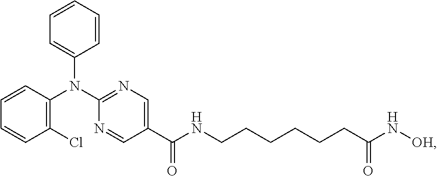 [SMILES: O=C(CCCCCCNC(=O)c1cnc(N(c2ccccc2)c2ccccc2Cl)nc1)NO]

CAY10603:

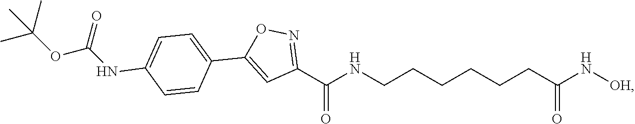 [SMILES: CC(C)(C)OC(=O)Nc1ccc(-c2cc(C(=O)NCCCCCCC(=O)NO)no2)cc1]

Tubastatin A:

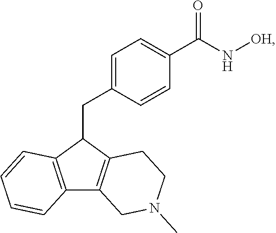 [SMILES: CN1CCC2=C(C1)c1ccccc1C2Cc1ccc(C(=O)NO)cc1]

HPOB:

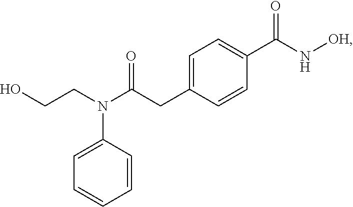 [SMILES: O=C(NO)c1ccc(CC(=O)N(CCO)c2ccccc2)cc1]

tubacin:

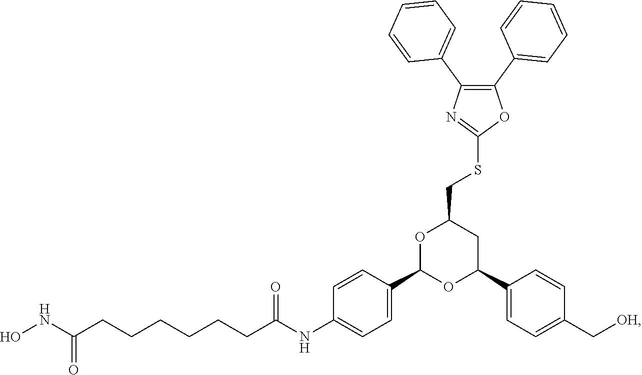 [SMILES: O=C(CCCCCCC(=O)Nc1ccc([C@H]2O[C@@H](CSc3nc(-c4ccccc4)c(-c4ccccc4)o3)C[C@@H](c3ccc(CO)cc3)O2)cc1)NO]

BATCP:

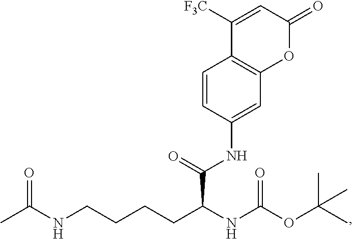 [SMILES: CC(=O)NCCCC[C@H](NC(=O)OC(C)(C)C)C(=O)Nc1ccc2c(C(F)(F)F)cc(=O)oc2c1]

panobinostat:

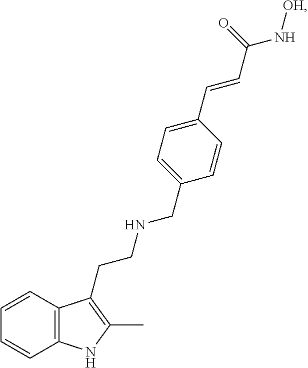 [SMILES: Cc1[nH]c2ccccc2c1CCNCc1ccc(/C=C/C(=O)NO)cc1]

and vorinostat:

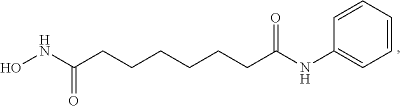 [SMILES: O=C(CCCCCCC(=O)Nc1ccccc1)NO]

and pharmaceutically acceptable salts, solvates, hydrates, cocrystals, or prodrugs thereof.

In an embodiment, the invention includes a method of treating a cancer in a human subject having a mutation in the ARID1A gene, comprising the step of administering a therapeutically effective dose of a HDAC6 inhibitor to the human subject in need thereof, further comprising the step of administering a second therapeutically effective dose of an enhancer of zeste homolog 2 (EZH2) inhibitor to the human subject.

In an embodiment, the invention includes a method of treating a cancer in a human subject having a mutation in the ARID1A gene, comprising the step of administering a therapeutically effective dose of a HDAC6 inhibitor to the human subject in need thereof, further comprising the step of administering a second therapeutically effective dose of EZH2 inhibitor to the human subject, wherein the HDAC6 inhibitor is administered to the subject concurrently with the administration of the EZH2 inhibitor.

In an embodiment, the invention includes a method of treating a cancer in a human subject having a mutation in the ARID1A gene, comprising the step of administering a therapeutically effective dose of a HDAC6 inhibitor to the human subject in need thereof, further comprising the step of administering a second therapeutically effective dose of EZH2 inhibitor to the human subject, wherein the HDAC6 inhibitor is administered to the subject before administration of the EZH2 inhibitor.

In an embodiment, the invention includes a method of treating a cancer in a human subject having a mutation in the ARID1A gene, comprising the step of administering a therapeutically effective dose of a HDAC6 inhibitor to the human subject in need thereof, further comprising the step of administering a second therapeutically effective dose of EZH2 inhibitor to the human subject, wherein the HDAC6 inhibitor is administered to the mammal after administration of the EZH2 inhibitor.

In an embodiment, the invention includes a method of treating a cancer in a human subject having a mutation in the ARID1A gene, comprising the step of administering a therapeutically effective dose of a HDAC6 inhibitor to the human subject in need thereof, further comprising the step of administering a second therapeutically effective dose of EZH2 inhibitor to the human subject, wherein the EZH2 inhibitor is selected from the group consisting of (S)-1-(sec-butyl)-N-((4,6-dimethyl-2-oxo-1,2-dihydropyridin-3-yl)methyl)-3-methyl-6-(6-(piperazin-1-yl)pyridin-3-yl)-1H-indole-4-carboxamide (GSK126):

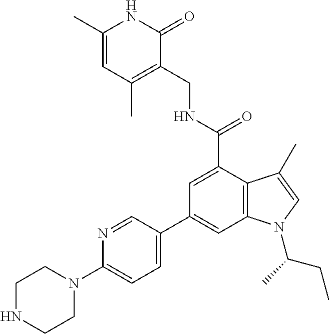 [SMILES: CC[C@H](C)n1cc(C)c2c(C(=O)NCc3c(C)cc(C)[nH]c3=O)cc(-c3ccc(N4CCNCC4)nc3)cc21]

tazemetostat:

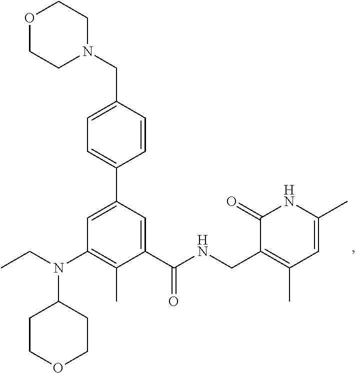 [SMILES: CCN(c1cc(-c2ccc(CN3CCOCC3)cc2)cc(C(=O)NCc2c(C)cc(C)[nH]c2=O)c1C)C1CCOCC1]

(R,Z)-1-(1-(1-(ethylsulfonyl)piperidin-4-yl)ethyl)-N-((2-hydroxy-4-methoxy-6-methylpyridin-3-yl)methyl)-2-methyl-1H-indole-3-carbimidic acid (CPI-169):

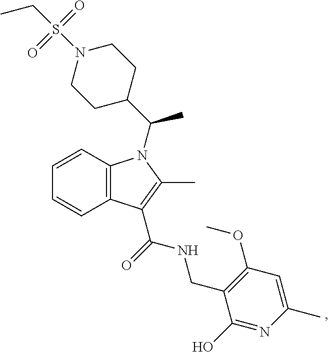 [SMILES: CCS(=O)(=O)N1CCC([C@@H](C)n2c(C)c(C(=O)NCc3c(OC)cc(C)nc3O)c3ccccc32)CC1]

1-cyclopentyl-N-((4,6-dimethyl-2-oxo-1,2-dihydropyridin-3-yl)methyl)-6-(4-(morpholinomethyl)phenyl)-1H-indazole-4-carboxamide (EPZ-5687):

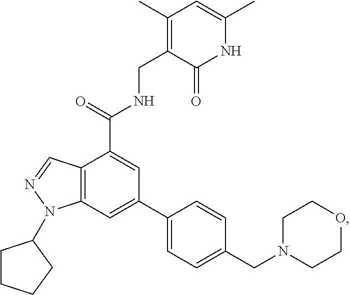 [SMILES: Cc1cc(C)c(CNC(=O)c2cc(-c3ccc(CN4CCOCC4)cc3)cc3c2cnn3C2CCCC2)c(=O)[nH]1]

N-((4,6-dimethyl-2-oxo-1,2-dihydropyridin-3-yl)methyl)-3-(ethyl((1R,4R)-4-((2-methoxyethyl)(methyl)amino)cyclohexyl)amino)-2-methyl-5-(3-morpholinoprop-1-yn-1-yl)benzamide (EPZ-11989):

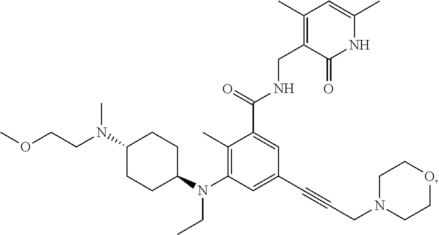 [SMILES: CCN(c1cc(C#CCN2CCOCC2)cc(C(=O)NCc2c(C)cc(C)[nH]c2=O)c1C)[C@H]1CC[C@H](N(C)CCOC)CC1]

1-isopropyl-N-((6-methyl-2-oxo-4-propyl-1,2-dihydropyridin-3-yl)methyl)-6-(2-(4-methylpiperazin-1-yOpyridin-4-yl)-1H-indazole-4-carboxamide (GSK343):

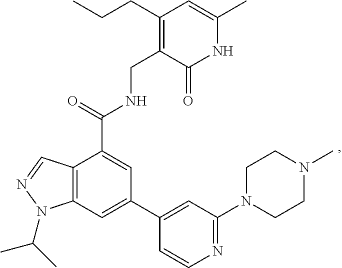 [SMILES: CCCc1cc(C)[nH]c(=O)c1CNC(=O)c1cc(-c2ccnc(N3CCN(C)CC3)c2)cc2c1cnn2C(C)C]

N-((4,6-dimethyl-2-oxo-1,2-dihydropyridin-3-yl)methyl)-1-isopropyl-3-methyl-6-(6-(4-methylpiperazin-1-yl)pyridin-3-yl)-1H-indole-4-carboxamide (GSK503):

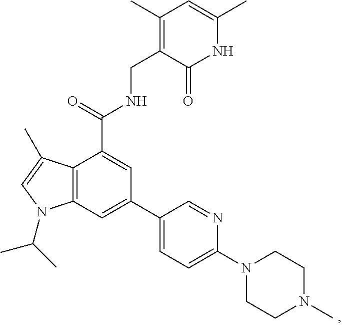 [SMILES: Cc1cc(C)c(CNC(=O)c2cc(-c3ccc(N4CCN(C)CC4)nc3)cc3c2c(C)cn3C(C)C)c(=O)[nH]1]

1-isopropyl-6-(6-(4-isopropylpiperazin-1-yl)pyridin-3-yl)-N-((6-methyl-2-oxo-4-propyl-1,2-dihydropyridin-3-yl)methyl)-1H-indazole-4-carboxamide (UNC-1999):

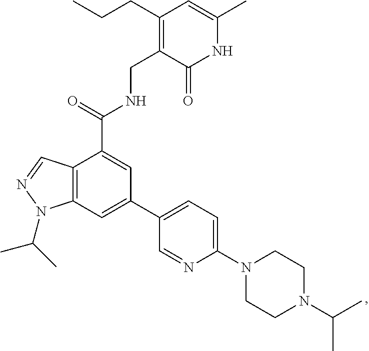 [SMILES: CCCc1cc(C)[nH]c(=O)c1CNC(=O)c1cc(-c2ccc(N3CCN(C(C)C)CC3)nc2)cc2c1cnn2C(C)C]

6-cyano-N-((4,6-dimethyl-2-oxo-1,2-dihydropyridin-3-yl)methyl)-1-(pentan-3-yl)-1H-indole-4-carboxamide (Ell):

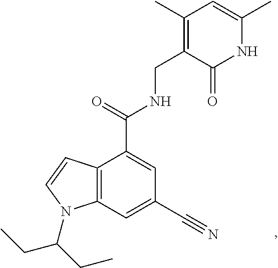 [SMILES: CCC(CC)n1ccc2c(C(=O)NCc3c(C)cc(C)[nH]c3=O)cc(C#N)cc21]

(1S,2R,5R)-5-(4-amino-1H-imidazo[4,5-c]pyridin-1-yl)-3-(hydroxymethyl)-3-cyclopentene-1,2-diol (DZNep):

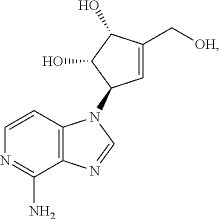 [SMILES: Nc1nccc2c1ncn2[C@@H]1C=C(CO)[C@@H](O)[C@H]1O]

sinefungin:

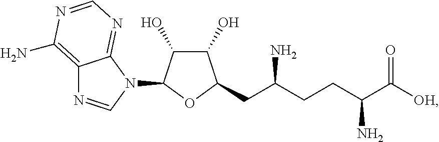 [SMILES: Nc1ncnc2c1ncn2[C@@H]1O[C@H](C[C@@H](N)CC[C@H](N)C(=O)O)[C@@H](O)[C@H]1O]

and pharmaceutically acceptable salts, solvates, hydrates, cocrystals, or prodrugs thereof.

### BRIEF DESCRIPTION OF THE DRAWINGS

The foregoing summary, as well as the following detailed description of the invention, will be better understood when read in conjunction with the appended drawings.

FIG. 1 illustrates expression of ARID1A as determined by immunoblot. GAPDH expression was used as a loading control. ARID1A wild-type RMG1 cells were transduced with lentivirus encoding shARID1A or control. ARID1A-inactivated cells are selectively sensitive to HDAC6 knockdown. FIG. 2, FIG. 3, and FIG. 4 show additional results of this series of experiments.

FIG. 2 illustrates mRNA expression. ARID1A knockdown or control RMG1 cells were transduced with lentivirus encoding shRNA to each of the 11 individual HDACs. RNA was isolated from the indicated cells and subjected to qRT-PCR for the indicated HDACs.

FIG. 3 illustrates a scatterplot of the integrated density normalized to control. The x-axis indicates changes in cell growth induced by individual shHDACs in control ARID1A wild-type treated cells, while the y-axis indicates changes in cell growth induced by the same shHDACs in shARID1A-expressing cells. The indicated cells were plated in 24-well plates in quadruplicates and subjected to colony formation assay for 12 days and stained with crystal violet. Integrated density was measured with NIH Image J software as a surrogate for cell growth.

FIG. 4 illustrates representative images of colony formation for the indicated shHDAC6 expressing cells.

FIG. 5 illustrates immunoblot results for a panel of ovarian clear cell carcinoma cell lines with known ARID1A mutational status were infected with lentivirus encoding two individual shHDAC6 or control. Knockdown of HDAC6 expression was determined by immunoblot. GAPDH expression was used as a loading control. HDAC6 inhibition selectively suppresses the growth of cells with ARID1A mutation.

FIG. 6 illustrates results from experiments performed as in FIG. 5, but the indicated cells were grown in 3D using Matrigel extracellular matrix for 12 days. Shown are representative images of acini formed by the indicated cells. Scale bar=75 AU determined by NIH Image J software.

FIG. 7 illustrates results from the experiments of FIG. 6, but diameters of 50 acini from each of the indicated groups were quantified. Error bars represent standard error of the mean.

FIG. 8 illustrates % growth results for the indicated ovarian clear cell carcinoma cell lines with known ARID1A mutational status, which were treated with the indicated concentration of HDAC6 inhibitor rocilinostat (ACY1215) or vehicle control in a colony formation assay. After 12 days culture, integrated density of colony formed by the indicated cells was quantified using NIH Image J software, and values were normalized to vehicle control. Data represent triplicates from independent experiments. Error bars represent S.E.M. The p-value was calculated via two-tailed t-test.

FIG. 9 illustrates the finding that ARID1A-mutated cells are more sensitive to the HDAC6 inhibitor CAY10603 compared with ARID1A wild-type cells. Indicated ovarian clear cell carcinoma cell lines with known ARID1A mutational status were treated with the indicated concentration of HDAC6 inhibitor CAY10603 or vehicle control in a colony formation assay. After 12 days culture, integrated density of colony formed by the indicated cells was quantified using NIH Image J software, and values were normalized to vehicle control. Data represent triplicates from independent experiments. Error bars represent S.E.M.

FIG. 10 illustrates the finding that HDAC6 inhibition induces apoptosis in ARID1A-inactivated cells. ARID1A-mutated TOV21G cells were treated with 1.25 μM of the HDAC6 inhibitor rocilinostat (ACY1215). Cells were collected and stained for apoptotic markers Annexin V. Apoptotic cells were quantified. Error bars represent standard error of the mean. The p-value was calculated via a two-tailed t-test.

FIG. 11 illustrates the results of the experiments of FIG. 10, but examined for apoptotic markers cleaved caspase 3 and cleaved PARP p85 by immunoblot. GAPDH expression was used as a loading control.

FIG. 12 shows that HDAC6 inhibitors induce apoptosis in ARID1A-mutated cells. ARID1A-mutated OVISE cells were treated with the HDAC6 inhibitor rocilinostat (ACY1215) (1.25 μM, 48 hours). Cells were collected, incubated with an Annexin V-FITC antibody and subjected to flow cytometry analysis. Annexin V-positive cells (apoptotic) were measured with FloJo software. Mean of three independent experiments with S.E.M.

FIG. 13 shows results as in FIG. 12, but for ARID1A wild-type RMG1 cells.

FIG. 14 shows results as in FIG. 12, but for ARID1A-mutated TOV21G cells treated with another HDAC6 inhibitor CAY10603.

FIG. 15 shows results as in FIG. 12, but for ARID1A-mutated OVISE cells treated with another HDAC6 inhibitor CAY10603.

FIG. 16 shows the results of ARID1A wild-type RMG1 cells expressing shARID1A or control treated with 1.25 μM rocilinostat (ACY1215). Expression of apoptotic markers cleaved caspase 3 and cleaved PARP p85 by immunoblot. GAPDH expression was used as a loading control.

FIG. 17 shows that apoptosis was also induced by HDAC6 knockdown in ARID1A knockdown cells but not in control ARID1A wild-type cells.

FIG. 18 shows that ARID1A represses HDAC6 expression. Expression of HDAC6 mRNA in ARID1A wild-type RMG1 cells with or without shARID1A expression were determined by qRT-PCR. Mean of three independent experiments with S.E.M. is shown.

FIG. 19 shows the results of the experiments as in FIG. 18, but examined for ARID1A and HDAC6 protein expression by immunoblot. GAPDH expression was used as a loading control.

FIG. 20 shows ARID1A mRNA expression in ARID1A-mutated TOV21G cells with or without wild-type ARID1A expression was examined by qRT-PCR.

FIG. 21 shows results for the experiments in FIG. 20, but for HDAC6 mRNA expression.

FIG. 22 shows results for the experiments in FIG. 20, but examined for ARID1A and HDAC6 protein expression by immunoblot. GAPDH expression was used as a loading control.

FIG. 23 illustrates representative images of immunohistochemical staining of ARID1A and HDAC6 on consecutive sections of endometrioid tumors developed from Apc^{-/-}/Pten^{-/-} or Apc^{-/-}/Pten^{-/-}/Arid1a^{-/-} conditional genetic mouse models. Scale bar=100 μm.

FIG. 24 demonstrates that ARID1A is enriched at the human HDAC6 gene promoter. ARID1A chromatin immunoprecipitation followed by next generation sequencing (ChIP-seq) and input tracks at the human HDAC6 gene promoter based on publically available ChIP-seq data (GSE69568).

FIG. 25 illustrates a quantification of the results of FIG. 23. Histological score (H-score) was calculated based on 5 separate fields for 3 different tumors from each of the indicated groups.

FIG. 26 shows the results of experiments wherein ARID1A wild-type RMG1 cells with or without ARID1A knockdown were subjected to chromatin immunoprecipitation (ChIP) analysis using anti-ARID1A antibodies. An isotype matched IgG was used as a negative control. The ChIP products were subjected to qPCR analysis using primers specific for the human HDAC6 gene promoter. Data is representative of 3 independent experiments. Error bars represent S.E.M. p-value calculated via two-tailed t-test.

FIG. 27 shows the results of experiments wherein ARID1A wild-type RMG1 cells with or without ARID1A knockdown were subjected to chromatin immunoprecipitation (ChIP) analysis using anti-PolII antibodies. An isotype matched IgG was used as a negative control. The ChIP products were subjected to qPCR analysis using primers specific for the human HDAC6 gene promoter. Data is representative of 3 independent experiments. Error bars represent S.E.M. p-value calculated via two-tailed t-test.

FIG. 28 illustrates that the selectivity of HDAC6 inhibition against ARID1A inactivation depends on p53 and HDAC6 deacetylase lysine 120 residues on p53. ARID1A-mutated TOV21G cells with or without shp53 expression were examined for p53 mRNA expression by qRT-PCR.

FIG. 29 illustrates results as in FIG. 28, but examined for p53 protein expression by immunoblot. GAPDH expression was used as a loading control.

FIG. 30 illustrates the results of treating the indicated cells with increasing does of the HDAC6 inhibitor rocilinostat (ACY1215) and assaying for growth in a colony formation assay for 12 days. Shown are representative images of colonies formed by the indicated cells.

FIG. 31 shows dose responsive curves of ARID1A-mutated TOV21G cells with or without p53 knockdown treated with the indicated concentration of rocilinostat (ACY1215) for 12 days in colony formation assay. Growth inhibition was calculated based on integrated density as measured in NIH ImageJ, and values were normalized to vehicle control. Data is representative of 3 independent experiments. Error bars represent S.E.M. The p-value was calculated via two-tailed t-test.

FIG. 32 illustrates that the selectivity of HDAC6 inhibitor against ARID1A-mutated cells is p53 dependent. ARID1A-mutated OVISE cells with or without shp53 expression were examined for p53 mRNA expression by qRT-PCR. Growth inhibition was calculated based on integrated density as measured in NIH ImageJ, and values were normalized to vehicle control. Data is representative of 3 independent experiments. Error bars represent S.E.M. The p-value was calculated via two-tailed t-test.

FIG. 33 illustrates results for the experiments of FIG. 32, but examined for p53 protein expression by immunoblot. GAPDH expression was used as a loading control.

FIG. 34 illustrates dose responsive curves of OVISE cells with or without p53 knockdown treated with the indicated concentration of rocilinostat (ACY1215) for 12 days in colony formation assay. Growth inhibition was calculated based on integrated density as measured in NIH ImageJ, and values were normalized to vehicle control. Data is representative of 3 independent experiments. Error bars represent S.E.M. p-value calculated via two-tailed t-test.

FIG. 35 illustrates the results of ARID1A-mutated TOV21G cells treated with vehicle control or the HDAC6 inhibitor rocilinostat (ACY1215) (1.25 μM). Expression of the indicated proteins was determined by immunoblot. GAPDH expression was used as a loading control.

FIG. 36 illustrates the results of analyzing ARID1A-mutated TOV21G cells expressing the indicated shHDAC6 or control for expression of p53K120Ac, total p65, and HDAC6 by immunoblot. GAPDH expression was used as a loading control.

FIG. 37 shows the results of treating ARID1A wild-type RMG1 with or without shARID1A expression with vehicle control or the HDAC6 inhibitor rocilinostat (ACY1215) (1.25 μM). Expression of the indicated proteins was examined by immunoblot. GAPDH expression was used as a loading control.

FIG. 38 shows the results of an in vitro deacetylase assay using purified human HDAC6 protein and a p53K120Ac containing peptide as a substrate. Deacetylation reactions were detected by discontinuous liquid chromatography-mass spectrometry (LC-MS). The negative control assay was run in the presence of the HDAC6 inhibitor rocilinostat (ACY1215). All assays were performed in triplicates.

FIG. 39 shows that HDAC6 inhibition decreases mitochondria membrane potential in an ARID1A status dependent manner. ARID1A-mutated TOV21G cells were treated with vehicle control, shControl, shHDAC6, rocilinostat (ACY1215, 1.25 μM), or CAY10603 (312 nM). RNA was extracted and utilized for next generation sequencing (RNA-seq). Expression of p53 target genes known to regulate apoptosis such as BAX, PUMA and NOXA, and known to regulate cell cycle arrest such as CDKN1A were not altered by HDAC6 inhibition in RNA-seq analysis.

FIG. 40 shows the results of ARID1A wild-type RMG1 cells with or without shARID1A expression treated with vehicle control (red), FCCP (positive control; blue) or rocilinostat (ACY1215, 1.25 μM; green). The mitochondria membrane potential was quantified by TMRE using FACS analysis. Data was collected via FACS and is representative of 3 independent experiments.

FIG. 41 shows a quantification of the results of FIG. 40. Error bars represent standard error mean. The p-value was calculated via two-tailed t-test.

FIG. 42 illustrates that apoptosis induced by HDAC6 inhibition in ARD1A-mutated cells correlates with mitochondrial localization of p53K120Ac and a decrease in mitochondrial membrane potential. ARID1A-mutated TOV21G cells were treated with vehicle control or the HDAC6 inhibitor rocilinostat (ACY1215, 1.25 μM) were fixed and subjected to immunofluorescence staining using antibodies against p53K120Ac (red), TOM20 (green, a mitochondrial marker) and DAPI (blue, nuclei). Images were captured using confocal microscopy.

FIG. 43 shows confocal images were processed and co-localization between p53K120AC and TOM20 was quantified using Lecia Application Suite X (LASX) software. Mean of three independent experiments with S.E.M.

FIG. 44 illustrates the results of treating ARID1A-mutated TOV21G cells with vehicle control or the HDAC6 inhibitor rocilinostat (ACY1215, 1.25 μM), which were fractionated to isolate mictochondria and cytosol. Expression of p53K120Ac, mitochondrial marker TOM20, and total p53 in the indicated fractions was examined by immunoblot. GAPDH expression was used as a loading control.

FIG. 45 shows the results as in FIG. 44, but examined for mitochondrial membrane potential by FACS.

FIG. 46 shows a quantification of the results of FIG. 45. Mean of five experimental replicates with S.E.M. p-value calculated via two-tailed t-test.

FIG. 47 shows a model for the mechanism by which HDAC6 inhibition promotes apoptosis in ARID1A-mutated cells.

FIG. 48 shows that HDAC6 inhibition significantly inhibits tumor growth ARID1A-mutated, but not wild-type, tumors. Luciferase-expressing ARID1A-mutated TOV21G cells were orthotopically transplanted into the ovarian bursa sac of SCID/nude female mice. Tumors were allowed to establish for 14 days before randomized into two groups (n=6/group). Mice were treated with vehicle control or HDAC6 inhibitor (rocilinostat/ACY1215, 50 mg/kg) daily for 21 days. Representative images of control and rocilinostat (ACY1215) treated mice at the end of treatment.

FIG. 49 shows additional results of the experiments shown in FIG. 48. During treatment at indicated time interval mice were imaged based on luciferase expression to monitor tumor growth. Total flux (photons/sec) is graphed. *p=0.0313.

FIG. 50 shows that HDAC6 inhibition improves the survival of mice bearing ARID1A-mutated ovarian tumors. ARID1A-mutated TOV21G cells were orthotopically transplanted into the ovarian bursa sac of SCID/nude female mice. Tumors were allowed to establish for 2 weeks before the mice were randomized into two different treatment groups (n=5/group). Mice were treated with vehicle control or the HDAC6 inhibitor rocilinostat (ACY1215) (50 mg/kg) daily for an additional 3 weeks. After stopping the treatment, the mice from the indicated groups were followed for survival. Shown is the Kaplan-Meier survival curves for ACY1215 or vehicle control treated mice. The p-value was calculated by log-rank test.

FIG. 51 shows results of the study in FIG. 50, but at the end of treatment, the mice were euthanized (n=6/group). Shown are representative images of reproductive tracts with tumors from control or rocilinostat (ACY1215) treated mice. Scale bar=2 cm.

FIG. 52 shows results of the study in FIG. 50, but the tumor weight was measured as a surrogate for tumor burden from the control and rocilinostat (ACY1215) treated mice.

FIG. 53 shows results of the study in FIG. 50, examined for disseminated tumor nodules in the peritoneal cavity. Representative images of disseminated tumor nodules in control and rocilinostat (ACY1215) treated mice. Asterisks (*) indicate the disseminated tumor nodules in peritoneal cavity.

FIG. 54 shows a quantification of the results in FIG. 53. The number of disseminated tumor nodules in peritoneal cavity was quantified.

FIG. 55 shows results as in FIG. 48 and FIG. 49, but with luciferase expressing ARID1A wild-type RMG1 cells.

FIG. 56 shows results as in FIG. 48 and FIG. 49, but with luciferase expressing ARID1A wild-type RMG1 cells.

FIG. 57 shows results as in FIG. 55, but the weight of tumors dissected from control and rocilinostat (ACY1215) treated mice was measured at the end of treatment as a surrogate for tumor burden.

FIG. 58 shows results as in FIG. 55, but counted for disseminated tumor nodules in the indicated treatment groups.

FIG. 59 shows consecutive sections of tumors dissected from the indicated treatment groups subjected to immunohistochemical staining for HDAC6, Ki67, cleaved caspase 3 and p53K120AC. Scale bar=100 μm.

FIG. 60 shows a quantification of the results in FIG. 59. Histological score (H-score) was calculated for 5 separate fields from 6 tumors from 6 individual mice from each of the indicated groups. Error bars represent S.E.M. The p-value was calculated via two-tailed t-test.

FIG. 61 shows results from the study in FIG. 51. The consecutive sections of tumors dissected from the indicated treatment groups were subjected to immunohistochemical staining for HDAC6, Ki67, cleaved caspase 3 and p53K120AC. Scale bar=100 μm.

FIG. 62 shows a quantification of the results in FIG. 59. Histological score (H-score) was calculated for 5 separate fields from 6 tumors from 6 individual mice from each of the indicated groups. Error bars represent S.E.M. The p-values were calculated via two-tailed t-test.

FIG. 63 illustrates Western blot results that show that a switch from BRG1 to BRM catalytic subunit underlies the up-regulation of anti-apoptotic HDAC6 in EZH2 inhibitor resistant cells. GSK126 was used as the EZH2 inhibitor in this study. Actin was used as a control.

FIG. 64 illustrates ARID1A protein expression in parental and ARID1A CRIPSR OVCA429 cells.

FIG. 65 illustrates a colony formation assay using the indicated OVCA429 cells with or without HDAC6 knockdown.

FIG. 66 illustrates the quantification of a colony formation assay using the indicated OVCA429 cells with or without HDAC6 knockdown.

FIG. 67 illustrates the expression of HDAC6, FLAG and a loading control β-actin in ARID1A-mutated TOV21G cells expressing a shHDAC and concurrent expression of FLAG-tagged shRNA resistant wildtype HDAC6 or a catalytically inactive H216/611A mutant; the indicated cells were subjected to colony formation assay and integrated density was measured.

FIG. 68 illustrates n=4 independent experiments described in FIG. 67.

FIG. 69 illustrates that the IC₅₀ of HDAC6 inhibitor ACY1215 is significantly higher in ARID1A wildtype than in mutated cells.

FIG. 70 illustrates the expression of ARID1A and a loading control β-actin in the indicated primary cultures of human ovarian clear cell carcinomas determined by immunoblot.

FIG. 71 illustrates the HDAC6 inhibitor ACY1215 dose response curves of primary clear cell ovarian tumour cultures with (VOA4841) and without (XVOA295) ARID1A expression; n=3 independent experiments.

FIG. 72 illustrates control and ARID1A CRISPR OVCA429 cells treated with or without 1.25 μM ACY1215 in a colony formation assay.

FIG. 73 illustrates the quantification of ARID1A CRISPR OVCA429 cells treated with or without 1.25 μM ACY1215 in a colony formation assay; n=4 independent experiments.

FIG. 74 illustrates percent apoptosis in various cell lines quantified by FACS based on Annexin V staining.

FIG. 75 illustrates percent apoptosis of in the indicated primary clear cell ovarian tumour cultures quantified by FACS based on Annexin V staining.

FIG. 76 illustrates expression of ARID1A and a loading control β-actin in TOV21G cells with or without wildtype ARID1A restoration.

FIG. 77 illustrates percent apoptosis based on Annexin V staining in ARID1A-mutated TOV21G cells with or without wildtype ARID1A restoration and treated with 1.25 μM ACY1215 or DMSO controls for 96 hrs.

FIG. 78 illustrates percent apoptosis based on Annexin V staining in ARID1A mutated TOV21G cells treated with 1.25 μM ACY1215, 20 μM pan-caspase inhibitor Q-VD-Oph, or a combination for 48 hrs.

FIG. 79 illustrates percent apoptosis based on Annexin V staining in ARID1A mutated TOV21G cells treated with 1.25 μM ACY1215 for 48 hrs with or without knockdown of caspase 3 or caspase 9.

FIG. 80 illustrates the human HDAC6 gene promoter activity in ARID1A wildtype RMG1 cells with or without ARID1A knockdown; n=3 independent experiments.

FIG. 81 illustrates ARID1A wildtype parental and knockout OVCA429 cells were examined for expression of HDAC6 mRNA; n=3 independent experiments.

FIG. 82 illustrates ARID1A wildtype parental and knockout OVCA429 cells were examined for or ARID1A, HDAC6 and (3-actin protein expression.

FIG. 83 illustrates the relative HDAC6 mRNA expression in ARID1A wildtype (n=12) and mutated (n=7) human ovarian clear cell carcinoma specimens; Mann-Whitney test was used to compare the two groups.

FIG. 84 illustrates ARID1A wildtype RMG1 cells with or without ARID1A knockdown were subjected to ChIP analysis for the HDAC6 gene promoter using BRG1; n=4 independent experiments; an isotype matched IgG was used as a control.

FIG. 85 illustrates ARID1A wildtype RMG1 cells with or without ARID1A knockdown were subjected to ChIP analysis for the HDAC6 gene promoter using Pol II; n=7 independent experiments; an isotype matched IgG was used as a control.

FIG. 86 illustrates ARID1A mutated TOV21G cells with or without wildtype ARID1A restoration were subjected to ChIP analysis for the HDAC6 gene promoter using antibodies against ARID1A; n=3 independent experiments; an isotype matched IgG was used as a control.

FIG. 87 illustrates ARID1A mutated TOV21G cells with or without wildtype ARID1A restoration were subjected to ChIP analysis for the HDAC6 gene promoter using anti-BRG1; n=4 independent experiments; an isotype matched IgG was used as a control.

FIG. 88 illustrates ARID1A mutated TOV21G cells with or without wildtype ARID1A restoration were subjected to ChIP analysis for the HDAC6 gene promoter using Pol II; n=4 independent experiments; an isotype matched IgG was used as a control.

FIG. 89 illustrates ARID1A-mutated TOV21G cells with or without p53 knockdown were examined for TP53 mRNA expression; n=3 independent experiments.

FIG. 90 illustrates ARID1A-mutated TOV21G cells with or without p53 knockdown were examined for p53 and GAPDH protein expression.

FIG. 91 illustrates dose responsive curves of the indicated cells treated with the HDAC6 inhibitor ACY1215; ARID1A wildtype RMG1 and OVCA429 cells were used as controls for comparison; n=4 independent experiments; error bars represent S.E.M. P-value calculated via two-tailed t-test.

FIG. 92 illustrates ARID1A-mutated TOV21G with or without p53 knockdown treated with ACY1215 (1.25 μM) or DMSO controls for 48 hours; percent apoptosis was quantified by FACS analysis based on Annexin V staining; n=3 independent experiments.

FIG. 93 illustrates ARID1A-mutated TOV21G treated with vehicle DMSO control or the HDAC6 inhibitor ACY1215 (1.25 μM); expression of the indicated proteins was determined.

FIG. 94 illustrates ARID1A mutated TOV21G cells were treated with vehicle DMSO control or the HDAC6 inhibitor ACY1215 (1.25 μM) were fractionated to isolate mitochondria and cytosol; expression of p53K120Ac, total p53, HDAC6, mitochondrial marker TOM20, and TIP60 that is known to acetylate p53K120 residue 31 in the indicated fractions was examined by immunoblot. GAPDH expression was used as a loading control.

FIG. 95 illustrates ARID1A-mutated TOV21G cells with or without endogenous p53 knockdown by a shp53 that targets the 3′ UTR region of the human TP53 gene together with a lentivirus encoding a control, wildtype p53 or a p53K12OR mutant—immunoblotting of p53 and GAPDH in the indicated cells.

FIG. 96 illustrates ARID1A-mutated TOV21G cells with or without endogenous p53 knockdown by a shp53 that targets the 3′ UTR region of the human TP53 gene together with a lentivirus encoding a control, wildtype p53 or a p53K12OR mutant—the indicated cells were treated with or without ACY1215 and examined for mitochondrial membrane potential by FACS; n=3 independent experiments.

FIG. 97 illustrates ARID1A-mutated TOV21G cells with or without endogenous p53 knockdown by a shp53 that targets the 3′ UTR 505 region of the human TP53 gene together with a lentivirus encoding a control, wildtype p53 or a p53K12OR mutant—the percentage of surviving cells of the indicated cells treated with ACY1215 was determined by colony formation assay; n=4 independent experiments.

FIG. 98 illustrates that HDAC6 knockdown showed the highest selectivity against ARID1A knockdown with the least growth inhibitory effects on controls.

FIG. 99 illustrates that HDAC6 knockdown was selective against ARID1A-mutated ovarian clear cell and endometrioid cancer cell lines in the Project Achilles synthetic lethality screen database.

FIG. 100 and FIG. 101 illustrate that the observed growth inhibition depends on the enzymatic activity of HDAC6 because the growth inhibition was rescued by a wildtype HDAC6 but not a catalytically inactive H216/611A mutant.

FIG. 102, FIG. 103, and FIG. 104 illustrate that the primary clear cell ovarian tumour cultures without ARID1A expression are more sensitive to ACY1215 compared to those with ARID1A expression; the IC50 values of ACY1215 in primary cells are comparable to those observed in cell lines; restoration of wildtype ARID1A in ARID1A-mutated TOV21G cells reduced the sensitivity of these cells to ACY1215.

FIG. 105 and FIG. 106 illustrate that ARID1A knockout significantly increased the sensitivity of ARID1A wildtype OVCA429 cells to ACY1215.

FIG. 107 illustrates that knockdown of other SWI/SNF subunits such as BRG1 1 did not increase ACY1215 sensitivity.

FIG. 108 illustrates that knockdown of other SWI/SNF subunits such as BRG1 1 did not increase ACY1215 sensitivity correlates with a compensation of BRG1 loss by the mutually exclusive catalytic subunit BRM.

FIG. 109 and FIG. 110 illustrate that a pan-caspase inhibitor Q-VD-Oph or knockdown of intrinsic apoptotic pathway initiator caspase 9 or effector caspase 3 significantly suppressed the apoptosis induced by ACY1215.

FIG. 111 and FIG. 112 illustrate that knockdown of Caspase 8, the caspase of the extrinsic apoptotic pathway 23, did not affect the apoptosis induced by ACY1215.

FIG. 113 illustrates that BRG1 knockdown did not affect repression of HDAC6 by wildtype ARID1A restoration in ARID1A-mutated cells.

FIG. 114 illustrates that HDAC6 is the only HDAC that is unregulated by ARID1A knockdown in ARID1A wildtype RMG1 cells and downregulated by wildtype ARID1A restoration in ARID1A mutated TOV21G cells.

FIG. 115 illustrates that cells derived from Apc−/−/Pten−/−/Arid1a−/− tumours are more sensitive to ACY1215 compared with those derived from Apc−/−/Pten−/− tumours.

FIG. 116 illustrates that HDAC6 was the only class II HDAC that is expressed at significantly higher levels in ARID1A-mutated compared to wildtype primary human clear cell ovarian carcinomas.

FIG. 117 and FIG. 118 illustrate that ARID1A expression negatively correlates with HDAC6 expression in both clear cell and endometrioid ovarian cancer cell lines and laser capture microdissected specimens based on database mining.

FIG. 119 and FIG. 122 illustrate that ARID1A knockdown reduced its association with the HDAC6 gene promoter, which correlated with a decrease in BRG1, an increase in RNA polymerase II (Pol II) and an increase in acetylated histone H3′s association with the HDAC6 gene promoter.

FIG. 120 and FIG. 121 illustrate that ARID1A knockdown reduced its association with the HDAC6 gene promoter, supporting the notion that ARID1A directly suppresses HDAC6 transcription.

FIG. 123 illustrates that knockdown of p53 expression significantly impaired the apoptosis and growth inhibition induced by the HDAC6 inhibitor CAY10603 in ARID1A-mutated TOV21G cells.

FIG. 124 and FIG. 125 illustrate that immunofluorescence analysis revealed that HDAC6 inhibition induced a significant increase in co-localization of p53K120Ac and the mitochondrial marker TOM20 or HDAC6.

FIG. 126 illustrates that ACY1215 significantly suppressed the tumour growth in the conditional Arid1a−/−/Pik3caH1047R genetic clear cell ovarian tumour mouse model.

### BRIEF DESCRIPTION OF THE SEQUENCE LISTING

SEQ ID NO:1 is a nucleotide sequence for a quantitative polymerase chain reaction (qPCR) primer.

SEQ ID NO:2 is a nucleotide sequence for a qPCR primer.

SEQ ID NO:3 is a nucleotide sequence for a qPCR primer.

SEQ ID NO:4 is a nucleotide sequence for a qPCR primer.

SEQ ID NO:5 is a nucleotide sequence for a qPCR primer.

SEQ ID NO:6 is a nucleotide sequence for a qPCR primer.

SEQ ID NO:7 is a nucleotide sequence for a qPCR primer.

SEQ ID NO:8 is a nucleotide sequence for a qPCR primer.

SEQ ID NO:9 is a nucleotide sequence for a qPCR primer.

SEQ ID NO:10 is a nucleotide sequence for a qPCR primer.

SEQ ID NO:11 is a nucleotide sequence for a qPCR primer.

SEQ ID NO:12 is a nucleotide sequence for a qPCR primer.

SEQ ID NO:13 is a nucleotide sequence for a qPCR primer.

SEQ ID NO:14 is a nucleotide sequence for a qPCR primer.

SEQ ID NO:15 is a nucleotide sequence for a qPCR primer.

SEQ ID NO:16 is a nucleotide sequence for a qPCR primer.

SEQ ID NO:17 is a nucleotide sequence for a qPCR primer.

SEQ ID NO:18 is a nucleotide sequence for a qPCR primer.

SEQ ID NO:19 is a nucleotide sequence for a qPCR primer.

SEQ ID NO:20 is a nucleotide sequence for a qPCR primer.

SEQ ID NO:21 is a nucleotide sequence for a qPCR primer.

SEQ ID NO:22 is a nucleotide sequence for a qPCR primer.

SEQ ID NO:23 is a nucleotide sequence for a qPCR primer.

SEQ ID NO:24 is a nucleotide sequence for a qPCR primer.

SEQ ID NO:25 is a nucleotide sequence for a qPCR primer.

SEQ ID NO:26 is a nucleotide sequence for a qPCR primer.

SEQ ID NO:27 is a nucleotide sequence for a qPCR primer.

SEQ ID NO:28 is a nucleotide sequence for a qPCR primer.

SEQ ID NO:29 is a nucleotide sequence for a qPCR primer.

SEQ ID NO:30 is a nucleotide sequence for a qPCR primer.

SEQ ID NO:31 is a nucleotide sequence for chromatin immunoprecipitation (ChIP).

SEQ ID NO:32 is a nucleotide sequence for ChIP.

SEQ ID NO:33 is the ARID1A gRNA sequence.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entireties.

### Definitions

The terms “co-administration,” “co-administering,” “administered in combination with,” “administering in combination with,” “simultaneous,” and “concurrent,” as used herein, encompass administration of two or more active pharmaceutical ingredients to a human subject so that both active pharmaceutical ingredients and/or their metabolites are present in the human subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present is also encompassed in the methods of the invention.

The terms “active pharmaceutical ingredient” and “drug” include EZH2 inhibitors and HDAC6 inhibitors.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the human subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit in a human subject. A prophylactic effect includes delaying or eliminating the appearance of a disease or condition, delaying or eliminating the onset of symptoms of a disease or condition, slowing, halting, or reversing the progression of a disease or condition, or any combination thereof.

The term “pharmaceutically acceptable salt” refers to salts derived from a variety of organic and inorganic counter ions known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid and phosphoric acid. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs, can also be incorporated into the described compositions and methods.

“Prodrug” is intended to describe a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers the advantages of solubility, tissue compatibility or delayed release in a mammalian organism (see, e.g., Bundgaard, Design of Prodrugs, Elsevier, Amsterdam, 1985). The term “prodrug” is also intended to include any covalently bonded carriers, which release the active compound in vivo when administered to a subject. Prodrugs of an active compound, as described herein, may be prepared by modifying functional groups present in the active compound in such a way that the modifications are cleaved, either in routine manipulation or in vivo, to yield the active parent compound. Prodrugs include, for example, compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetates, formates and benzoate derivatives of an alcohol, various ester derivatives of a carboxylic acid, or acetamide, formamide and benzamide derivatives of an amine functional group in the active compound.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this invention.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, from 0% to 10%, from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Alkyl” refers to a straight or branched hydrocarbon chain radical consisting solely of carbon and hydrogen atoms, containing no unsaturation, having from one to ten carbon atoms (e.g., (C₁-₁₀)alkyl or C₁-₁₀ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocycyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂-₁₀)alkenyl or C₂-₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂-₁₀)alkynyl or C₂-₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(Ra)₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C₃-₁₀)cycloalkyl or C₃-₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R)₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a (C₁-₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R)₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R₂ of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4] dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Nitro” refers to the —NO₂ radical.

“Oxa” refers to the —O— radical.

“Oxo” refers to the ═O radical.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In some embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or enantiomers can be prepared by asymmetric syntheses. See, for example, Jacques et al. Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1R)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, 3rd Edition, John Wiley & Sons, New York (1999).

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)-(optionally substituted heteroaryl) and —S(O)-(optionally substituted heterocycloalkyl).

“Sulfonyl” refers to groups that include —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

Compounds used in the methods of the invention also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

### Methods of Treating Cancers and Other Diseases

The compositions and methods described herein can be used in a method for treating diseases. In an embodiment, they are for use in treating hyperproliferative disorders. They may also be used in treating other disorders as described herein and in the following paragraphs.

In some embodiments, the hyperproliferative disorder is cancer. In some embodiments, the hyperproliferative disorder is an ARID1A-mutated cancer. In some embodiments, the ARID1A-mutated cancer is selected from the group consisting of ovarian cancer, non-small-cell lung cancer, and renal cancer. In some embodiments, the ARID1A-mutated cancer is selected from the group consisting of gastric cancer, hepatocellular cancer, breast cancer, ovarian clear cell carcinoma, uterine endometriod carcinoma, uterine clear cell carcinoma, pancreatic cancer, transitional cell carcinoma of the bladder, Waldenstrom's macroglobulinemia, anplastic thyroid carcinoma, colon cancer, lung cancer, cervical adenocarcinoma, bile duct carcinoma, prostate cancer, and medulloblastoma.

In an embodiment, the hyperproliferative disorder is EZH2-mutated cancer. EZH2-mutated cancers are described, e.g., in WO 2015/128837 A1, the disclosure of which is incorporated by reference herein. EZH2-mutated cancers include point mutations, such as the alanine-to-valine mutation at residue 687 of EZH2 (the A687V mutation).

Efficacy of the compounds and combinations of compounds described herein in treating, preventing and/or managing the indicated diseases or disorders can be tested using various models known in the art, which provide guidance for treatment of human disease. For example, models for determining efficacy of treatments for ovarian cancer are described, e.g., in Mullany et al. Endocrinology 2012, 153, 1585-92; and Fong et al. J. Ovarian Res. 2009, 2, 12. Models for determining efficacy of treatments for pancreatic cancer are described in Herreros-Villanueva et al. World J. Gastroenterol. 2012, 18, 1286-1294. Models for determining efficacy of treatments for breast cancer are described, e.g., in Fantozzi, Breast Cancer Res. 2006, 8, 212. Models for determining efficacy of treatments for melanoma are described, e.g., in Damsky et al. Pigment Cell & Melanoma Res. 2010, 23, 853-859. Models for determining efficacy of treatments for lung cancer are described, e.g., in Meuwissen et al. Genes & Development, 2005, 19, 643-664. Models for determining efficacy of treatments for lung cancer are described, e.g., in Kim, Clin. Exp. Otorhinolaryngol. 2009, 2, 55-60; and Sano, Head Neck Oncol. 2009, 1, 32. Models for determining efficacy in B cell lymphomas, such as diffuse large B cell lymphoma (DLBCL), include the PiBCL1 murine model with BALB/c (haplotype H-2d) mice. Illidge et al. Cancer Biother. & Radiopharm. 2000, 15, 571-80. Efficacy of treatments for Non-Hodgkin's lymphoma may be assessed using the 38C13 murine model with C3H/HeN (haplotype 2-Hk) mice or alternatively the 38C13 Her2/neu model. Timmerman et al. Blood 2001, 97, 1370-77; Penichet et al. Cancer Immunolog. Immunother. 2000, 49, 649-662. Efficacy of treatments for chronic lymphocytic leukemia (CLL) may be assessed using the BCL1 model using BALB/c (haplotype H-2d) mice. Dutt et al. Blood 2011, 117, 3230-29.

### HDAC6 Inhibitors

In an embodiment, the invention includes a method of treating a cancer in a human subject suffering from an ARID1A mutated cancer, the method comprising the step of administering a therapeutically effective dose of an HDAC6 inhibitor to the human subject. The HDAC6 inhibitor may be any HDAC6 inhibitor known in the art, including selective HDAC6 inhibitors and pan-HDAC inhibitors that inhibit HDAC6 as well as other HDACs. Suitable HDAC6 inhibitors are described, for example, in West and Johnstone, J. Clin. Invest. 2014, 124, 30-39; Mottamal et al. Molecules 2015, 20, 3898-3941. In particular, the HDAC6 inhibitor is an HDAC6 inhibitor described in more detail in the following paragraphs.

In an embodiment, the HDAC6 inhibitor is rocilinostat, also known as ACY-1215 or ACY1215 (Acetylon Pharmaceuticals, Inc.), which has the chemical name 2-(diphenylamino)-N-(7-(hydroxyamino)-7-oxoheptyl)pyrimidine-5-carboxamide (Formula (1)):

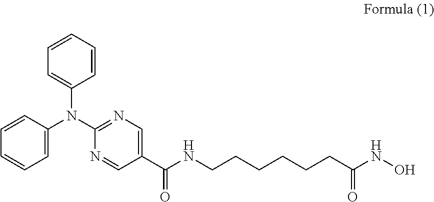 [SMILES: O=C(CCCCCCNC(=O)c1cnc(N(c2ccccc2)c2ccccc2)nc1)NO]

or a pharmaceutically acceptable salt, solvate, hydrate, cocrystal, tautomer, or prodrug thereof. Rocilinostat is commercially available. The synthesis and properties of rocilinostat and other suitable HDAC6 inhibitors are described in, e.g., U.S. Pat. Nos. 8,148,526; 8,394,810; and 8,609,678; the disclosures of which are incorporated by reference herein.

In an embodiment, the HDAC6 inhibitor is ACY-241 or ACY241 (Acetylon Pharmaceuticals, Inc.), which has the chemical name (Formula (2)):

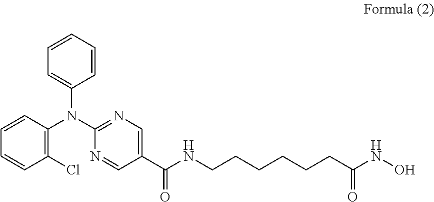 [SMILES: O=C(CCCCCCNC(=O)c1cnc(N(c2ccccc2)c2ccccc2Cl)nc1)NO]

or a pharmaceutically acceptable salt, solvate, hydrate, cocrystal, tautomer, or prodrug thereof. ACY-241 is commercially available. The synthesis and properties of ACY-241 and other suitable HDAC6 inhibitors are described in, e.g., U.S. Pat. Nos. 8,148,526; 8,394,810; and 8,609,678; the disclosures of which are incorporated by reference herein.

In an embodiment, the HDAC6 inhibitor is a compound of Formula (3):

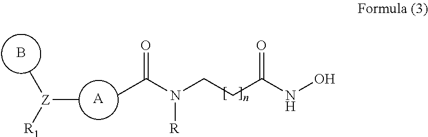

or a pharmaceutically acceptable salt, solvate, hydrate, cocrystal, tautomer, or prodrug thereof, wherein:
Z is N or CR*, wherein R* is an optionally substituted alkyl, an optionally substituted acyl, an optionally substituted aryl or an optionally substituted heteroaryl;
ring A is an optionally substituted aryl or an optionally substituted heteroaryl;
ring B is an optionally substituted aryl or an optionally substituted heteroaryl;
R₁ is (i) H, alkyl, haloalkyl, alkenyl, aryl, arylalkyl, heteroaryl, heterocyclic, carbocyclic, C(O)—R₂, C(O)O—R₂, or S(O)ₚ, each of which may be optionally substituted; or (ii) when Z is CR*, R₁ may be optionally substituted branched alkyl, OR₃, or N(R₃)(R₃), —CH₂CH₂OH, OCH₂CH₂OH, SH, or thio alkoxy;
or ring B and R₁ may together with the atom to which each is attached, form an optionally substituted heterocyclic, or an optionally substituted heteroaryl;
or R* and R₁together with the atom to which each is attached, may form an optionally substituted carbocyclic, optionally substituted heterocyclic, optionally substituted aryl or optionally substituted heteroaryl ring;
R is H or an optionally substituted alkyl; or R and ring A may be joined to form a fused bicyclic ring which may be optionally substituted;
each R₂ is independently alkyl, cycloalkyl, heterocycloalkyl, aryl, or heteroaryl, each of which is optionally substituted;
each R₃ is independently alkyl, cycloalkyl, heterocycloalkyl, aryl, or heteroaryl, each of which is optionally substituted;
n is 4, 5, 6, 7 or 8; and
p is 0, 1, or 2.

In one embodiment, the ring A is phenyl, naphthyl, anthracenyl, pyridinyl, pyrimidinyl, pyrazinyl, indolyl, imidazolyl, oxazolyl, furyl, thienyl, thiazolyl, triazolyl, isoxazolyl, quinolinyl, pyrrolyl, pyrazolyl, or 5,6,7,8-tetrahydroisoquinoline; each of which may be optionally substituted.

In another embodiment, the ring B is phenyl, naphthyl, anthracenyl, pyridinyl, pyrimidinyl, pyrazinyl, indolyl, imidazolyl, oxazolyl, furyl, thienyl, thiazolyl, triazolyl, isoxazolyl, quinolinyl, pyrrolyl, pyrazolyl, or 5,6,7,8-tetrahydroisoquinoline; each of which may be optionally substituted.

In other embodiments, R₁ is H, optionally substituted alkyl, optionally substituted aryl, or optionally substituted heteroaryl, or R₁ is OH or alkoxy.

In some embodiments, R₁ is H, methyl, ethyl, propyl, i-propyl, butyl, i-butyl, t-butyl, pentyl, hexyl, phenyl, naphthyl, pyridinyl, OH, OCH₃, OCH₂CH₃, O—Pr, O-iPr, O—Bu, O-sBu, or O-tBu; each of which may be optionally substituted.

In some embodiments, R₁ is OH, alkoxy, NH₂, NH(alkyl), N(alkyl)(alkyl), NH-aryl, NH-hetroaryl, N(aryl)(aryl), N(aryl)(heteroaryl), or N(heteroaryl)(heteroaryl).

In some embodiments, the carbonyl and the Z group attached to ring A are disposed para to each other.

In some embodiments, the carbonyl and Z group attached to ring A are disposed meta to each other.

In some embodiments, the carbonyl and the Z group attached to ring A are disposed ortho to each other.

The synthesis and properties of compounds of Formula (3) and other suitable HDAC6 inhibitors are described in, e.g., U.S. Pat. Nos. 8,148,526; 8,394,810; and 8,609,678; the disclosures of which are incorporated by reference herein.

In an embodiment, the HDAC6 inhibitor is a compound of Formula (4):

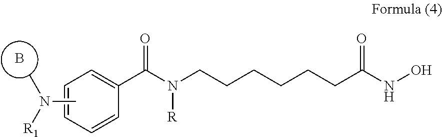

or a pharmaceutically acceptable salt, solvate, hydrate, cocrystal, tautomer, or prodrug thereof, wherein:
ring B is an optionally substituted aryl or an optionally substituted heteroaryl;
R₁ is H, alkyl, haloalkyl, alkenyl, aryl, arylalkyl, heteroaryl, heterocyclic, carbocyclic, C(O)—R₂, or C(O)O—R₂, each of which may be optionally substituted;
R₂ is optionally substituted heteroaryl, and
R is H or an optionally substituted alkyl; or R and the phenyl ring may be joined to form a fused [6,5] bicyclic ring which may be optionally substituted.

In some embodiments, ring B is phenyl, pyridinyl, pyrimidinyl, or pyrazinyl; each of which may be optionally substituted.

In some embodiments, ring B is substituted by alkyl, aryl, aralkyl, haloalkyl, hal, OH, NH₂, CN, or NO₂.

In some embodiments, R₁ is H, alkyl, aryl, arylalkyl, heteroaryl, C(O)—R₂, or C(O)O—R₂, each of which may be optionally substituted.

In some embodiments, R₂ is optionally substituted pyridinyl.

The synthesis and properties of compounds of Formula (4) and other suitable HDAC6 inhibitors are described in, e.g., U.S. Pat. Nos. 8,148,526; 8,394,810; and 8,609,678; the disclosures of which are incorporated by reference herein.

In an embodiment, the HDAC6 inhibitor is CAY10603, which has the chemical name tert-butyl 4-(3-((7-(hydroxyamino)-7-oxoheptyl)carbamoy)isoxazol-5-yl)phenylcarbamate (Formula (5)):

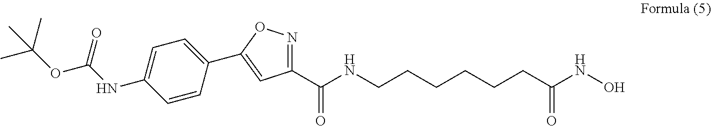 [SMILES: CC(C)(C)OC(=O)Nc1ccc(-c2cc(C(=O)NCCCCCCC(=O)NO)no2)cc1]

or a pharmaceutically acceptable salt, solvate, hydrate, cocrystal, tautomer, or prodrug thereof. CAY10603 is commercially available.

In an embodiment, the HDAC6 inhibitor is Tubastatin A, which has the chemical name N-hydroxy-4-((2-methyl-2,3,4,5-tetrahydro-1H-indeno[1,2-c]pyridin-5-yl)methyl)benzamide (Formula (6)):

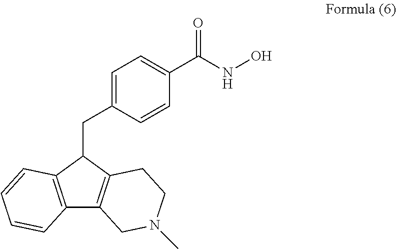 [SMILES: CN1CCC2=C(C1)c1ccccc1C2Cc1ccc(C(=O)NO)cc1]

or a pharmaceutically acceptable salt, solvate, hydrate, cocrystal, tautomer, or prodrug thereof. Tubastatin A is commercially available.

In an embodiment, the HDAC6 inhibitor is HPOB, which has the chemical name N-hydroxy-4-(2-((2-hydroxyethyl)(phenyl)amino)-2-oxoethyl)benzamide (Formula (7)):

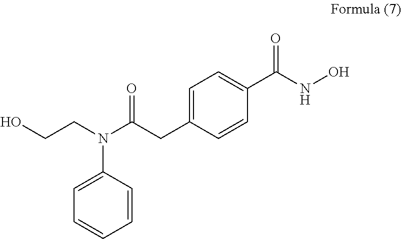 [SMILES: O=C(NO)c1ccc(CC(=O)N(CCO)c2ccccc2)cc1]

or a pharmaceutically acceptable salt, solvate, hydrate, cocrystal, tautomer, or prodrug thereof. HPOB is commercially available.

In an embodiment, the HDAC6 inhibitor is tubacin, which has the chemical name N1-(4-((2R,4R,6S)-4-((4,5-diphenyloxazol-2-ylthio)methyl)-6-(4-(hydroxymethyl)phenyl)-1,3-dioxan-2-yl)phenyl)-N8-hydroxyoctanediamide (Formula (8)):

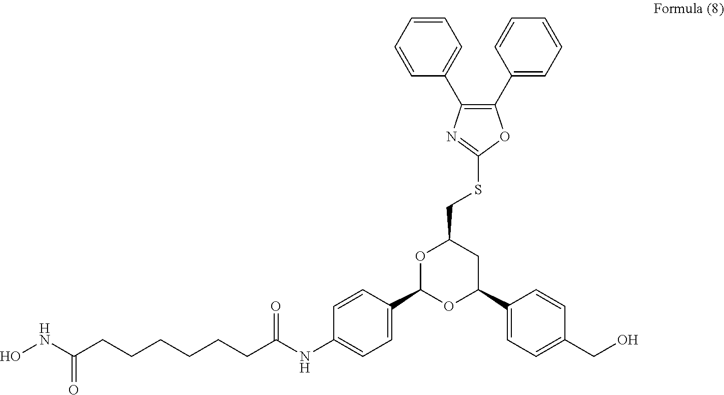 [SMILES: O=C(CCCCCCC(=O)Nc1ccc([C@H]2O[C@@H](CSc3nc(-c4ccccc4)c(-c4ccccc4)o3)C[C@@H](c3ccc(CO)cc3)O2)cc1)NO]

or a pharmaceutically acceptable salt, solvate, hydrate, cocrystal, tautomer, or prodrug thereof. Tubacin is commercially available.

In an embodiment, the HDAC6 inhibitor is BATCP, which has the chemical name (S)-[5-Acetylamino-1-(2-oxo-4-trifluoromethyl-2H-chromen-7-ylcarbamoyl)pentyl]carbamic acid tert-butyl ester (Formula (9)):

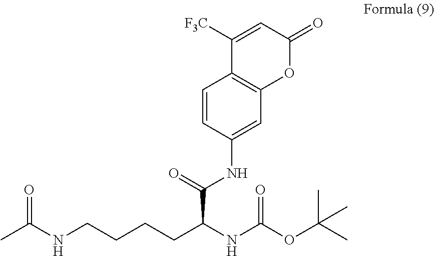 [SMILES: CC(=O)NCCCC[C@H](NC(=O)OC(C)(C)C)C(=O)Nc1ccc2c(C(F)(F)F)cc(=O)oc2c1]

or a pharmaceutically acceptable salt, solvate, hydrate, cocrystal, tautomer, or prodrug thereof. BATCP is commercially available.

In an embodiment, the HDAC6 inhibitor is panobinostat, which has the chemical name (2E)-N-hydroxy-3-[4-({[2-(2-methyl-1H-indol-3-yl)ethyl]amino}methyl)phenyl]acrylamide (Formula (10)):

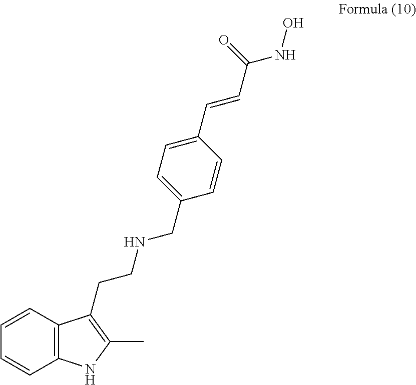 [SMILES: Cc1[nH]c2ccccc2c1CCNCc1ccc(/C=C/C(=O)NO)cc1]

or a pharmaceutically acceptable salt, solvate, hydrate, cocrystal, tautomer, or prodrug thereof. Panobinostat is commercially available. The synthesis and properties of compounds of Formula (10) and other suitable HDAC6 inhibitors are described in, e.g., U.S. Pat. No. 6,833,384; the disclosure of which is incorporated by reference herein.

In an embodiment, the HDAC6 inhibitor is vorinostat, which has the chemical name N-hydroxy-N′-phenyloctanediamide (Formula (11)):

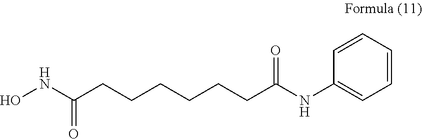 [SMILES: O=C(CCCCCCC(=O)Nc1ccccc1)NO]

or a pharmaceutically acceptable salt, solvate, hydrate, cocrystal, tautomer, or prodrug thereof. Vorinostat is commercially available. The synthesis and properties of compounds of Formula (11) and other suitable HDAC6 inhibitors are described in, e.g., U.S. Pat. No. RE 38,506; the disclosure of which is incorporated by reference herein.

In an embodiment, the HDAC6 inhibitor is ACY-775, which has the chemical name 2-((1-(3-fluorophenyl)cyclohexyl)amino)-N-hydroxypyrimidine-5-carboxamide, or a pharmaceutically acceptable salt, solvate, hydrate, cocrystal, tautomer, or prodrug thereof (Formula 12; Celgene Corp):

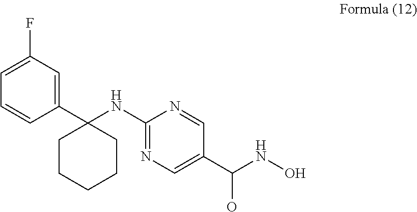 [SMILES: ONC(O)c1cnc(NC2(c3cccc(F)c3)CCCCC2)nc1]

In an embodiment, the HDAC6 inhibitor is ACY-738, which has the chemical name N-hydroxy-2-(1-phenylcycloproylamino)pyrimidine-5-carboxamide, or a pharmaceutically acceptable salt, solvate, hydrate, cocrystal, tautomer, or prodrug thereof (Formula 13, Celgene Corp):

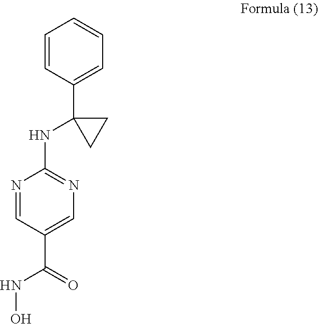 [SMILES: O=C(NO)c1cnc(NC2(c3ccccc3)CC2)nc1]

In an embodiment, the HDAC6 inhibitor is Nexturastat A, which has the chemical name 4-[[butyl(phenylcarbamoyl)amino]methyl]-N-hydroxybenzamide, or a pharmaceutically acceptable salt, solvate, hydrate, cocrystal, tautomer, or prodrug thereof (Formula 14, H. Lee Moffitt Cancer Center & Research Institute Inc):

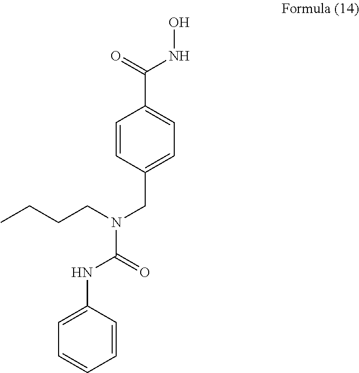 [SMILES: CCCCN(Cc1ccc(C(=O)NO)cc1)C(=O)Nc1ccccc1]

In an embodiment, the HDAC6 inhibitor is ACY-1083, or a pharmaceutically acceptable salt, solvate, hydrate, cocrystal, tautomer, or prodrug thereof (Celgene Corp). In an embodiment, the HDAC6 inhibitor is CKD-506, or a pharmaceutically acceptable salt, solvate, hydrate, cocrystal, tautomer, or prodrug thereof (Chong Kun Dang Pharmaceutical Corp). In an embodiment, the HDAC6 inhibitor is CKD-504, or a pharmaceutically acceptable salt, solvate, hydrate, cocrystal, tautomer, or prodrug thereof (Chong Kun Dang Pharmaceutical Corp). In an embodiment, the HDAC6 inhibitor is CKD-509, or a pharmaceutically acceptable salt, solvate, hydrate, cocrystal, tautomer, or prodrug thereof (Chong Kun Dang Pharmaceutical Corp). In an embodiment, the HDAC6 inhibitor is QTX-125, or a pharmaceutically acceptable salt, solvate, hydrate, cocrystal, tautomer, or prodrug thereof (Quimatryx SL). In an embodiment, the HDAC6 inhibitor is QTX-153, or a pharmaceutically acceptable salt, solvate, hydrate, cocrystal, tautomer, or prodrug thereof (Quimatryx SL). In an embodiment, the HDAC6 inhibitor is KA-2507, or a pharmaceutically acceptable salt, solvate, hydrate, cocrystal, tautomer, or prodrug thereof (Karus Therapeutics Ltd). In an embodiment, the HDAC6 inhibitor is SP-259, or a pharmaceutically acceptable salt, solvate, hydrate, cocrystal, tautomer, or prodrug thereof (Shuttle Pharmaceuticals LLC). In an embodiment, the HDAC6 inhibitor is KAN-0439221, or a pharmaceutically acceptable salt, solvate, hydrate, cocrystal, tautomer, or prodrug thereof (Kancera AB).

### EZH2 Inhibitors

In an embodiment, the invention includes a method of treating a cancer in a human subject suffering from an ARID1A mutated cancer, the method comprising the step of administering a therapeutically effective dose of an HDAC6 inhibitor and a therapeutically effective dose of an EZH2 inhibitor to the human subject. The EZH2 inhibitor may be any EZH2 inhibitor known in the art. Suitable EZH2 inhibitors are described, for example, in Momparler and Côté, Expert Opin. Investig. Drugs 2015, 24, 1031-43. In particular, the EZH2 inhibitor is an EZH2 inhibitor described in more detail in the following paragraphs. Synthetic lethality by targeting EZH2 methyltransferase activity in ARID1A-mutated cancers, and the relevance of EZH2 and ARID1A mutations for the epigenetic synthetic lethality in ovarian clear cell carcinoma, has been described in the art by Bitler et al. Nat. Med. 2015, 21, 231-238, and Bitler et al. Molecular & Cellular Oncology 2016, 3:1, e1032476, the contents of both incorporated herein by reference in their entireity.

In an embodiment, the EZH2 inhibitor is (S)-1-(sec-butyl)-N-((4,6-dimethyl-2-oxo-1,2-dihydropyridin-3-yl)methyl)-3-methyl-6-(6-(piperazin-1-yl)pyridin-3-yl)-1H-indole-4-carboxamide, also known as GSK2816126 or GSK126 (Formula (15)):

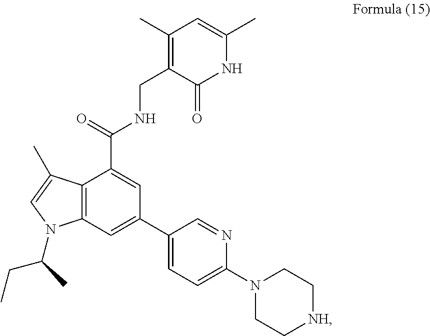 [SMILES: CC[C@H](C)n1cc(C)c2c(C(=O)NCc3c(C)cc(C)[nH]c3=O)cc(-c3ccc(N4CCNCC4)nc3)cc21]

or a pharmaceutically acceptable salt, solvate, hydrate, cocrystal, tautomer, or prodrug thereof. GSK126 is commercially available from multiple suppliers. The synthesis and properties of GSK126 and other suitable EZH2 inhibitors are described in, e.g., U.S. Pat. Nos. 8,536,179, 8,846,935, and 8,637,509, the disclosures of which are incorporated by reference herein.

In an embodiment, the EZH2 inhibitor is a compound of Formula (16):

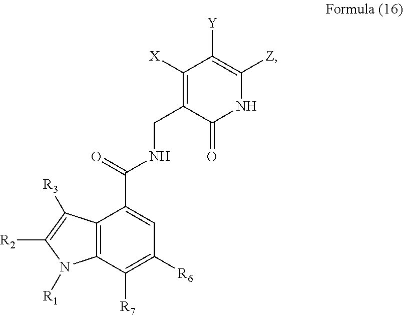

or a pharmaceutically acceptable salt, solvate, hydrate, cocrystal, tautomer, or prodrug thereof, wherein:
X and Z are selected independently from the group consisting of hydrogen, (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₅-C₈)cycloalkenyl-(C₁-C₈)alkyl or —(C₂-C₈)alkenyl, (C₆-C₁₀)bicycloalkyl, unsubstituted or substituted heterocycloalkyl, unsubstituted or substituted heterocycloalkyl-(C₁-C₈)alkyl or —(C₂-C₈)alkenyl, unsubstituted or substituted aryl, unsubstituted or substituted aryl-(C₁-C₈)alkyl or —(C₂-C₈)alkenyl, unsubstituted or substituted heteroaryl, unsubstituted or substituted heteroaryl-(C₁-C₈)alkyl or —(C₂-C₈)alkenyl, halo, cyano, —COR^{a}, —CO₂R^{a}, —CONR^{a}R^{b}, —CONR^{a}NR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, and —OC(O)NR^{a}R^{b};
Y is H or halo;
R₁ is (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₅-C₈)cycloalkenyl-(C₁-C₈)alkyl or —(C₂-C₈)alkenyl, unsubstituted or substituted (C₆-C₁₀)bicycloalkyl, unsubstituted or substituted heterocycloalkyl or —(C₂-C₈)alkenyl, unsubstituted or substituted heterocycloalkyl-(C₁-C₈)alkyl, unsubstituted or substituted aryl, unsubstituted or substituted aryl-(C₁-C₈)alkyl or —(C₂-C₈)alkenyl, unsubstituted or substituted heteroaryl, unsubstituted or substituted heteroaryl-(C₁-C₈)alkyl or —(C₂-C₈)alkenyl, —COR^{a}, —CO₂R^{a}, —CONR^{a}R^{b}, —CONR^{a}NR^{a}R^{b};
R₂ is hydrogen, (C₁-C₈)alkyl, trifluoromethyl, alkoxy, or halo, in which said (C₁-C₈)alkyl maybe substituted with one to two groups selected from: amino, and (C₁-C₃)alkylamino;
R₃ is hydrogen, (C₁-C₈)alkyl, cyano, trifluoromethyl, —NR^{a}R^{b}, or halo;
R₆ is selected from the group consisting of hydrogen, halo, (C₁-C₈)alkyl, (C₂-C₈)alkenyl, —B(OH)₂, substituted or unsubstituted (C₂-C₈)alkynyl, unsubstituted or substituted (C₃-C₈)cycloalkyl, unsubstituted or substituted (C₃-C₈)cycloalkyl-(C₁-C₈)alkyl, unsubstituted or substituted (C₅-C₈)cycloalkenyl, unsubstituted or substituted (C₅-C₈)cycloalkenyl-(C₁-C₈)alkyl, (C₆-C₁₀)bicycloalkyl, unsubstituted or substituted heterocycloalkyl, unsubstituted or substituted heterocycloalkyl-(C₁-C₈)alkyl, unsubstituted or substituted aryl, unsubstituted or substituted aryl-(C₁-C₈)alkyl, unsubstituted or substituted heteroaryl, unsubstituted or substituted heteroaryl-(C₁-C₈)alkyl, cyano, —COR^{a}, —CO₂R^{a}, —CONR^{a}R^{b}, —CONR^{a}NR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR′SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, —OC(O)NR^{a}R^{b};
R₇ is hydrogen, (C₁-C₃)alkyl, or alkoxy;
wherein any (C₁-C₈)alkyl, (C₂-C₈)alkenyl, (C₂-C₈)alkynyl, cycloalkyl, cycloalkenyl, bicycloalkyl, heterocycloalkyl, aryl, or heteroaryl group is optionally substituted by 1, 2 or 3 groups independently selected from the group consisting of —O(C₁-C₆)alkyl(R^{c})₁₋₂, —S(C₁-C₆)alkyl(R^{c})₁₋₂, —(C₁-C₆)alkyl(R^{c})₁₋₂, (C₁-C₈)alkyl-heterocycloalkyl, (C₃-C₈)cycloalkyl-heterocycloalkyl, halo, (C₁-C₆)alkyl, (C₃-C₈)cycloalkyl, (C₅-C₈)cycloalkenyl, (C₁-C₆)haloalkyl, cyano, —COR^{a}, —CO₂R^{a}, —CONR^{a}R^{b}, —SR^{a}, —SOW, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR′SO₂NR^{a}R^{b}, —OR^{a}, —OC(O)R^{a}, —OC(O)NR^{a}R^{b}, heterocycloalkyl, aryl, heteroaryl, aryl(C₁-C₄)alkyl, and heteroaryl(C₁-C₄)alkyl;
wherein any aryl or heteroaryl moiety of said aryl, heteroaryl, aryl(C₁-C₄)alkyl, or heteroaryl(C₁-C₄)alkyl is optionally substituted by 1, 2 or 3 groups independently selected from the group consisting of halo, (C₁-C₆)alkyl, (C₃-C₈)cycloalkyl, (C₅-C₈)cycloalkenyl, (C₁-C₆)haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, and —OC(O)NR^{a}R^{b};
R^{a} and R^{b} are each independently hydrogen, (C₁-C₈)alkyl, (C₂-C₈)alkenyl, (C₂-C₈)alkynyl, (C₃-C₈)cycloalkyl, (C₅-C₈)cycloalkenyl, (C₆-C₁₀)bicycloalkyl, heterocycloalkyl, aryl, heteroaryl, wherein said (C₁-C₈)alkyl, (C₂-C₈)alkenyl, (C₂-C₈)alkynyl, cycloalkyl, cycloalkenyl, bicycloalkyl, heterocycloalkyl, aryl or heteroaryl group is optionally substituted by 1, 2 or 3 groups independently selected from halo, hydroxyl, (C₁-C₄)alkoxy, amino, (C₁-C₄)alkylamino, ((C₁-C₄)alkyl)((C₁-C₄)alkyl)amino, —CO₂H, —CO₂(C₁-C₄)alkyl, —CONH₂, —CONH(C₁-C₄)alkyl, —CON((C₁-C₄)alkyl)((C₁-C₄)alkyl), —SO₂(C₁-C₄)alkyl, —SO₂NH₂, —SO₂NH(C₁-C₄)alkyl, or —SO₂N((C₁-C₄)alkyl)((C₁-C₄)alkyl);
each R^{c} is independently (C₁-C₄)alkylamino, —NR^{a}SO₂R^{b}, —SOW, —SO₂R^{a}, —NR^{a}C(O)OR^{a}, —NR^{a}R^{b}, or —CO₂R^{a};
or R^{a} and R^{b} taken together with the nitrogen to which they are attached represent a 5-8 membered saturated or unsaturated ring, optionally containing an additional heteroatom selected from oxygen, nitrogen, and sulfur, wherein said ring is optionally substituted by 1, 2 or 3 groups independently selected from (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, and (C₁-C₄)alkoxy(C₁-C₄)alkyl, wherein said ring is optionally fused to a (C₃-C₈)cycloalkyl, heterocycloalkyl, aryl, or heteroaryl ring;
or R^{a} and R^{b} taken together with the nitrogen to which they are attached represent a 6- to 10-membered bridged bicyclic ring system optionally fused to a (C₃-C₈)cycloalkyl, heterocycloalkyl, aryl, or heteroaryl ring.

The synthesis and properties of these and other suitable EZH2 inhibitors are described in, e.g., U.S. Pat. Nos. 8,536,179, 8,846,935, and 8,637,509, the disclosures of which are incorporated by reference herein.

In an embodiment, the EZH2 inhibitor is N-((4,6-dimethyl-2-oxo-1,2-dihydropyridin-3-yl)methyl)-1-isopropyl-3-methyl-6-(6-(4-methylpiperazin-1-yl)pyridin-3-yl)-1H-indole-4-carboxamide, also known as GSK503 (Formula (17)):

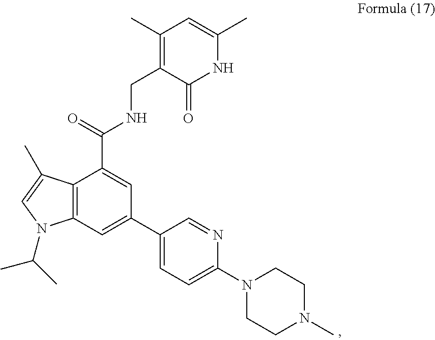 [SMILES: Cc1cc(C)c(CNC(=O)c2cc(-c3ccc(N4CCN(C)CC4)nc3)cc3c2c(C)cn3C(C)C)c(=O)[nH]1]

or a pharmaceutically acceptable salt, solvate, hydrate, cocrystal, tautomer, or prodrug thereof. The synthesis and properties of Formula (3) are described in, e.g., U.S. Pat. Nos. 8,536,179, 8,846,935, and 8,637,509, the disclosures of which are incorporated by reference herein.

In an embodiment, the EZH2 inhibitor is 1-isopropyl-N-((6-methyl-2-oxo-4-propyl-1,2-dihydropyridin-3-yl)methyl)-6-(2-(4-methylpiperazin-1-yl)pyridin-4-yl)-1H-indazole-4-carboxamide, also known as GSK343 (Formula (18)):

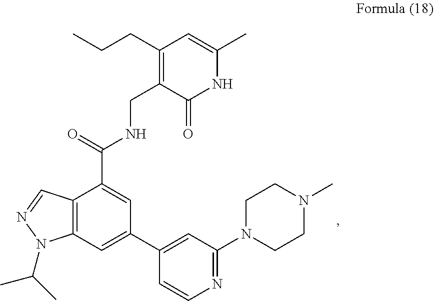 [SMILES: CCCc1cc(C)[nH]c(=O)c1CNC(=O)c1cc(-c2ccnc(N3CCN(C)CC3)c2)cc2c1cnn2C(C)C]

or a pharmaceutically acceptable salt, solvate, hydrate, cocrystal, tautomer, or prodrug thereof. The synthesis and properties of Formula (4), and other EZH2 inhibitors suitable for use with the present methods, are described in, e.g., U.S. Pat. Nos. 8,637,509, 8,846,935, and 9,018,382, the disclosures of which are incorporated by reference herein.

In an embodiment, the EZH2 inhibitor is N-((4,6-dimethyl-2-oxo-1,2-dihydropyridin-3-yl)methyl)-5-(ethyl(tetrahydro-2H-pyran-4-yl)amino)-4-methyl-4′-(morpholinomethyl)-[1,1′-biphenyl]-3-carboxamide, also known as tazemetostat or EPZ-6438 (Formula (19)):

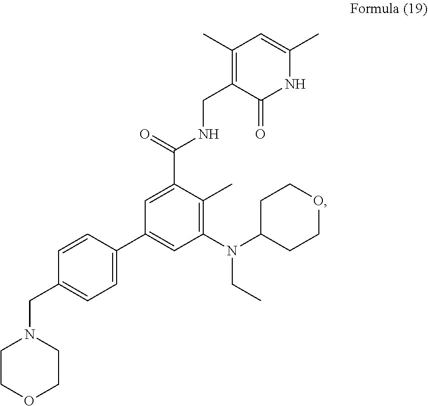 [SMILES: CCN(c1cc(-c2ccc(CN3CCOCC3)cc2)cc(C(=O)NCc2c(C)cc(C)[nH]c2=O)c1C)C1CCOCC1]

or a pharmaceutically acceptable salt, solvate, hydrate, cocrystal, tautomer, or prodrug thereof. Tazemetostat is commercially available from Epizyme, Inc., and is described in Knutson et al. Mol. Cancer Ther. 2014, 13, 842-54. The synthesis and properties of tazemetostat and other suitable EZH2 inhibitors are described in, e.g., U.S. Pat. Nos. 8,765,732, 8,410,088, and 9,090,562, the disclosures of which are incorporated by reference herein.

In an embodiment, the EZH2 inhibitor is a compound of Formula (20):

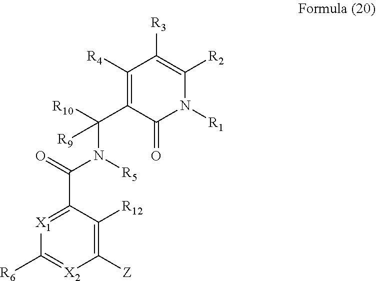

or a pharmaceutically acceptable salt, solvate, hydrate, cocrystal, tautomer, or prodrug thereof, wherein:
X₁ is N or CR₁₁;
X₂ is N or CR₁₃;
Z is NR₇R₈, OR_{S}, S(O)ₙR₇, or CR₇R₈R₁₄, in which n is 0, 1, or 2;
each of R₁, R₅, R₉, and R₁₀, independently, is H or C₁-C₆ alkyl optionally substituted with one or more substituents selected from the group consisting of halo, hydroxyl, COOH, C(O)O—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl;
each of R₂, R₃, and R₄, independently, is -Q₁-T₁, in which Q₁ is a bond or C₁-C₃ alkyl linker optionally substituted with halo, cyano, hydroxyl or C₁-C₆ alkoxy, and T₁ is H, halo, hydroxyl, COOH, cyano, or R_{S1}, in which R_{S1} is C₁-C₃ alkyl, C₂-C₆ alkenyl, C₂-C₆ alkynyl, C₁-C₆alkoxyl, C(O)O—C₁-C₆ alkyl, C₃-C₈ cycloalkyl, C₆-C₁₀ aryl, amino, mono-C₁-C₆ alkylamino, di-C₁-C₆ alkylamino, 4 to 12-membered heterocycloalkyl, or 5- or 6-membered heteroaryl, and R_{S1} is optionally substituted with one or more substituents selected from the group consisting of halo, hydroxyl, oxo, COOH, C(O)O—C₁-C₆ alkyl, cyano, C₁-C₆alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl;
R₆ is C₆-C₁₀ aryl or 5- or 6-membered heteroaryl, each of which is optionally substituted with one or more -Q₂-T₂, wherein Q₂ is a bond or C₁-C₃ alkyl linker optionally substituted with halo, cyano, hydroxyl or C₁-C₆ alkoxy, and T₂ is H, halo, cyano, —ORₐ, —NRₐR_{b}, —(NRₐR_{b}R_{c})⁺A⁻, —C(O)Rₐ, —C(O)ORₐ, —C(O)NRₐR_{b}, —NR_{b}C(O)Rₐ, —NR_{b}C(O)ORₐ, —S(O)₂Rₐ, —S(O)₂NRₐR_{b}, or R_{S2}, in which each of Rₐ, R_{b}, and R_{c}, independently is H or R_{S3}, A⁻ is a pharmaceutically acceptable anion, each of R_{S2} and R_{S3}, independently, is C₁-C₆ alkyl, C₃-C₈ cycloalkyl, C₆-C₁₀ aryl, 4 to 12-membered heterocycloalkyl, or 5- or 6-membered heteroaryl, or Rₐ and R_{b}, together with the N atom to which they are attached, form a 4 to 12-membered heterocycloalkyl ring having 0 or 1 additional heteroatom, and each of R_{S2}, R_{S3}, and the 4 to 12-membered heterocycloalkyl ring formed by Rₐ and R_{b}, is optionally substituted with one or more one or more -Q₃-T₃, wherein Q₃ is a bond or C₁-C₃ alkyl linker each optionally substituted with halo, cyano, hydroxyl or C₁-C₆ alkoxy, and T₃ is selected from the group consisting of halo, cyano, C₁-C₆ alkyl, C₃-C₈ cycloalkyl, C₆-C₁₀ aryl, 4 to 12-membered heterocycloalkyl, 5- or 6-membered heteroaryl, OR_{d}, COOR_{d}, —S(O)₂R_{d}, —NR_{d}Rₑ, and —C(O)NR_{d}Rₑ, each of R_{d} and Rₑ independently being H or C₁-C₆ alkyl, or -Q₃-T₃ is oxo; or any two neighboring -Q₂-T₂, together with the atoms to which they are attached form a 5- or 6-membered ring optionally containing 1-4 heteroatoms selected from N, O and S and optionally substituted with one or more substituents selected from the group consisting of halo, hydroxyl, COOH, C(O)O—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl;
R₇ is -Q₄-T₄, in which Q₄ is a bond, C₁-C₄ alkyl linker, or C₂-C₄ alkenyl linker, each linker optionally substituted with halo, cyano, hydroxyl or C₁-C₆ alkoxy, and T₄ is H, halo, cyano, NR_{f}R_{g}, —OR_{f}, —C(O)R_{f}, —C(O)OR_{f}, —C(O)NR_{f}R_{g}, —C(O)NR_{f}OR_{g}, —NR/C(O)R_{g}, —S(O)₂R_{f}, or R_{S4}, in which each of R_{f} and R_{g}, independently is H or R_{S5}, each of R_{S4} and R_{S5}, independently is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl, and each of R_{S4} and R_{S5} is optionally substituted with one or more -Q₅-T₅, wherein Q₅ is a bond, C(O), C(O)NRₖ, NRₖC(O), S(O)₂, or C₁-C₃ alkyl linker, Rₖ being H or C₁-C₆ alkyl, and T₅ is H, halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-Cᵢₒ aryl, 4 to 12-membered heterocycloalkyl, 5- or 6-membered heteroaryl, or S(O)_{q}R_{q} in which q is 0, 1, or 2 and R_{q} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl, and T₅ is optionally substituted with one or more substituents selected from the group consisting of halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl except when T₅ is H, halo, hydroxyl, or cyano; or -Q₅-T₅ is oxo;
each of R₈, R₁₁, R₁₂, and R₁₃, independently, is H, halo, hydroxyl, COOH, cyano, R_{S6}, OR_{S6}, or COOR_{S6}, in which R_{S6} is C₁-C₆ alkyl, C₂-C₆ alkenyl, C₂-C₆ alkynyl, C₃-C₈ cycloalkyl, 4 to 12-membered heterocycloalkyl, amino, mono-C₁-C₆ alkylamino, or di-C₁-C₆ alkylamino, and R_{S6} is optionally substituted with one or more substituents selected from the group consisting of halo, hydroxyl, COOH, C(O)O—C₁-C₆ alkyl, cyano, C₁-C₆ alkoxyl, amino, mono-C₁-C₆ alkylamino, and di-C₁-C₆ alkylamino; or R₇ and R₈, together with the N atom to which they are attached, form a 4 to 11-membered heterocycloalkyl ring having 0 to 2 additional heteroatoms, or R₇ and R₈, together with the C atom to which they are attached, form C₃-C₈ cycloalkyl or a 4 to 11-membered heterocycloalkyl ring having 1 to 3 heteroatoms, and each of the 4 to 11-membered heterocycloalkyl rings or C₃-C₈ cycloalkyl formed by R₇ and R₈ is optionally substituted with one or more -Q₆-T₆, wherein Q₆ is a bond, C(O), C(O)NRₘ, NRₘC(O), S(O)₂, or C₁-C₃ alkyl linker, Rₘ being H or C₁-C₆ alkyl, and T₆ is H, halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, 5- or 6-membered heteroaryl, or S(O)ₚRₚ in which p is 0, 1, or 2 and Rₚ is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl, and T₆ is optionally substituted with one or more substituents selected from the group consisting of halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl except when T₆ is H, halo, hydroxyl, or cyano; or -Q₆-T₆ is oxo; and
R₁₄ is absent, H, or C₁-C₆ alkyl optionally substituted with one or more substituents selected from the group consisting of halo, hydroxyl, COOH, C(O)O—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.
   The synthesis and properties of the EZH2 inhibitors of Formula (6) and other suitable EZH2 inhibitors are described in, e.g., U.S. Pat. Nos. 8,765,732, 8,410,088, and 9,090,562, the disclosures of which are incorporated by reference herein.

In an embodiment, the EZH2 inhibitor is (R,Z)-1-(1-(1-(ethylsulfonyl)piperidin-4-yl)ethyl)-N-((2-hydroxy-4-methoxy-6-methylpyridin-3-yl)methyl)-2-methyl-1H-indole-3-carbimidic acid, also known as CPI-169 (Formula (21)):

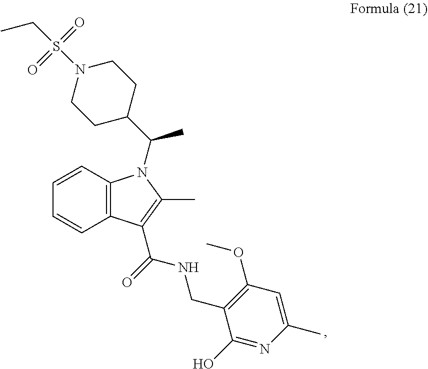 [SMILES: CCS(=O)(=O)N1CCC([C@@H](C)n2c(C)c(C(=O)NCc3c(OC)cc(C)nc3O)c3ccccc32)CC1]

or a pharmaceutically acceptable salt, solvate, hydrate, cocrystal, tautomer, or prodrug thereof. The synthesis and properties of Formula (7) and other suitable EZH2 inhibitors are described in, e.g., U.S. Patent Application Publication No. US 2016/0009718 A1, the disclosures of which are incorporated by reference herein.

In an embodiment, the EZH2 inhibitor is 1-cyclopentyl-N-((4,6-dimethyl-2-oxo-1,2-dihydropyridin-3-yl)methyl)-6-(4-(morpholinomethyl)phenyl)-1H-indazole-4-carboxamide, also known as EPZ-5687 (Formula (22)):

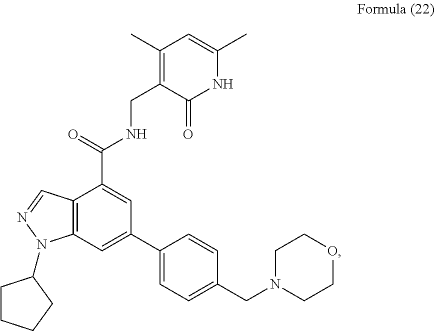 [SMILES: Cc1cc(C)c(CNC(=O)c2cc(-c3ccc(CN4CCOCC4)cc3)cc3c2cnn3C2CCCC2)c(=O)[nH]1]

or a pharmaceutically acceptable salt, solvate, hydrate, cocrystal, tautomer, or prodrug thereof.

In an embodiment, the EZH2 inhibitor is N-((4,6-dimethyl-2-oxo-1,2-dihydropyridin-3-yl)methyl)-3-(ethyl((1R,4R)-4-((2-methoxyethyl)(methyl)amino)cyclohexyl)amino)-2-methyl-5-(3-morpholinoprop-1-yn-1-yl)benzamide, also known as EPZ-11989 (Formula (23)):

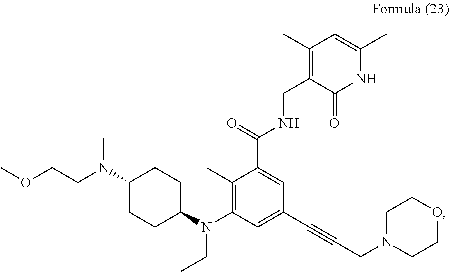 [SMILES: CCN(c1cc(C#CCN2CCOCC2)cc(C(=O)NCc2c(C)cc(C)[nH]c2=O)c1C)[C@H]1CC[C@H](N(C)CCOC)CC1]

or a pharmaceutically acceptable salt, solvate, hydrate, cocrystal, tautomer, or prodrug thereof.

In an embodiment, the EZH2 inhibitor is 1-isopropyl-6-(6-(4-isopropylpiperazin-1-yl)pyridin-3-yl)-N-((6-methyl-2-oxo-4-propyl-1,2-dihydropyridin-3-yl)methyl)-1H-indazole-4-carboxamide, also known as UNC-1999 (Formula (24)):

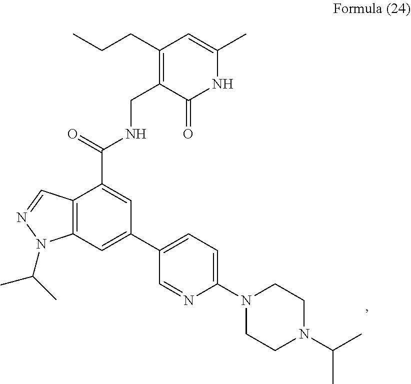 [SMILES: CCCc1cc(C)[nH]c(=O)c1CNC(=O)c1cc(-c2ccc(N3CCN(C(C)C)CC3)nc2)cc2c1cnn2C(C)C]

or a pharmaceutically acceptable salt, solvate, hydrate, cocrystal, tautomer, or prodrug thereof.

In an embodiment, the EZH2 inhibitor is 6-cyano-N-((4,6-dimethyl-2-oxo-1,2-dihydropyridin-3-yl)methyl)-1-(pentan-3-yl)-1H-indole-4-carboxamide, also known as Ell (Formula (25)):

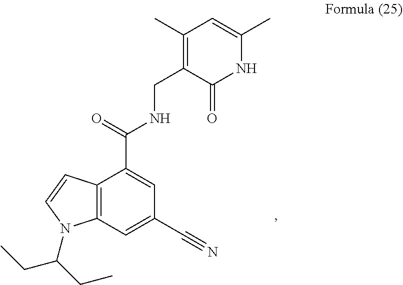 [SMILES: CCC(CC)n1ccc2c(C(=O)NCc3c(C)cc(C)[nH]c3=O)cc(C#N)cc21]

or a pharmaceutically acceptable salt, solvate, hydrate, cocrystal, tautomer, or prodrug thereof. The synthesis and properties of Ell are described in, e.g., Qi et al. Proc. Natl. Acad. Sci. USA 2012, /09, 21360-65.

In an embodiment, the EZH2 inhibitor is (1S,2R,5R)-5-(4-amino-1H-imidazo[4,5-c]pyridin-1-yl)-3-(hydroxymethyl)-3-cyclopentene-1,2-diol, also known as DZNep (Formula (26)):

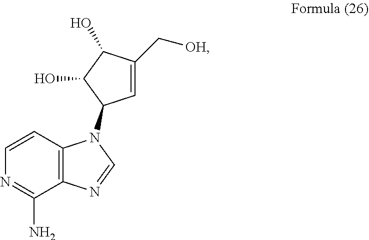 [SMILES: Nc1nccc2c1ncn2[C@@H]1C=C(CO)[C@@H](O)[C@H]1O]

or a pharmaceutically acceptable salt, solvate, hydrate, cocrystal, tautomer, or prodrug thereof.

In an embodiment, the EZH2 inhibitor is (2S,5S)-2,5-diamino-6-((2R,3S,4R,5R)-5-(6-amino-9H-purin-9-yl)-3,4-dihydroxytetrahydrofuran-2-yl)hexanoic acid, also known as 5′-deoxy-5′-(1,4-diamino-4-carboxybutyl)adenosine and sinefungin (Formula (27)):

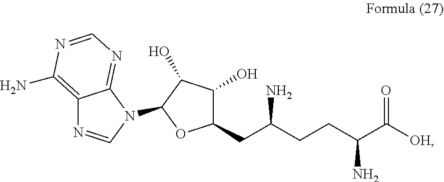 [SMILES: Nc1ncnc2c1ncn2[C@@H]1O[C@H](C[C@@H](N)CC[C@H](N)C(=O)O)[C@@H](O)[C@H]1O]

or a pharmaceutically acceptable salt, solvate, hydrate, cocrystal, tautomer, or prodrug thereof. The isolation of sinefungin is described, e.g., in U.S. Pat. No. 3,758,681, the disclosure of which is incorporated by reference herein. The synthesis of sinefungin is described, e.g., in Maguire et al. J. Org. Chem. 1990, 55, 948. In an embodiment, the EZH2 inhibitor is a derivative of sinefungin. Sinefungin derivatives, and are described, e.g., in French Patent No. FR 2664277 B1 and in Zheng et al. J. Am. Chem. Soc. 2012, 134, 18004-14, the disclosures of which are incorporated by reference herein.

In an embodiment, the EZH2 inhibitor is CPI-1205, or a pharmaceutically acceptable salt, solvate, hydrate, cocrystal, tautomer, or prodrug thereof. CPI-1205 is available from Constellation Pharmaceuticals.

### Pharmaceutical Compositions

In an embodiment, an active pharmaceutical ingredient or combination of active pharmaceutical ingredients, such as any of the foregoing HDAC6 or EZH2 inhibitors, is provided as a pharmaceutically acceptable composition.

In some embodiments, the concentration of each of the active pharmaceutical ingredients provided in the pharmaceutical compositions of the invention, such as any of the foregoing HDAC6 or EZH2 inhibitors, is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of each of the active pharmaceutical ingredients provided in the pharmaceutical compositions of the invention, such as any of the foregoing HDAC6 or EZH2 inhibitors, is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of each of the active pharmaceutical ingredients provided in the pharmaceutical compositions of the invention, such as any of the foregoing HDAC6 or EZH2 inhibitors, is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of each of the active pharmaceutical ingredients provided in the pharmaceutical compositions of the invention, such as any of the foregoing HDAC6 or EZH2 inhibitors, is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of each of the active pharmaceutical ingredients provided in the pharmaceutical compositions of the invention, such as any of the foregoing HDAC6 or EZH2 inhibitors, is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of each of the active pharmaceutical ingredients provided in the pharmaceutical compositions of the invention, such as any of the foregoing HDAC6 or EZH2 inhibitors, is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each of the active pharmaceutical ingredients according to the invention is effective over a wide dosage range. For example, in the treatment of adult humans, dosages independently range from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician. The clinically-established dosages of the foregoing HDAC6 or EZH2 inhibitors may also be used if appropriate.

In an embodiment, the molar ratio of two active pharmaceutical ingredients in the pharmaceutical compositions is in the range from 10:1 to 1:10, from 2.5:1 to 1:2.5, and about 1:1. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20.

In an embodiment, the pharmaceutical compositions of the present invention, such as any of the foregoing HDAC6 or EZH2 inhibitors, are for use in the treatment of cancers associated ARID1A mutations. In an embodiment, the pharmaceutical compositions of the present invention are for use in the treatment of a cancer associated with ARID1A mutations selected from the group consisting of bladder cancer, squamous cell carcinoma including head and neck cancer, pancreatic ductal adenocarcinoma, pancreatic cancer, colon carcinoma, mammary carcinoma, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, esophageal cancer, testicular cancer, gynecological cancer, thyroid cancer, aquired immune deficiency syndrome (AIDS)-related lymphoma, Kaposi's sarcoma, viral-induced cancer, glioblastoma, esophogeal tumors, hematological neoplasms, non-small-cell lung cancer, chronic myelocytic leukemia, diffuse large B-cell lymphoma, esophagus tumor, follicle center lymphoma, head and neck tumor, hepatitis C virus infection, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

Described below are non-limiting pharmaceutical compositions and methods for preparing the same.

### Pharmaceutical Compositions for Oral Administration

In some embodiments, the invention provides a pharmaceutical composition for oral administration containing the active pharmaceutical ingredient or combination of active pharmaceutical ingredients, such as the HDAC6 or EZH2 inhibitors described herein, and a pharmaceutical excipient suitable for oral administration.

In some embodiments, the invention provides a solid pharmaceutical composition for oral administration containing: (i) an effective amount of an active pharmaceutical ingredient or combination of active pharmaceutical ingredients, and (ii) a pharmaceutical excipient suitable for oral administration. In selected embodiments, the composition further contains (iii) an effective amount of a third active pharmaceutical ingredient and optionally (iv) an effective amount of a fourth active pharmaceutical ingredient.

In some embodiments, the pharmaceutical composition may be a liquid pharmaceutical composition suitable for oral consumption. Pharmaceutical compositions of the invention suitable for oral administration can be presented as discrete dosage forms, such as capsules, sachets, or tablets, or liquids or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The invention further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Each of the active pharmaceutical ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmacetical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present invention and to minimize precipitation of the compound of the present invention. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In some embodiments, the invention provides a pharmaceutical composition for injection containing an active pharmaceutical ingredient or combination of active pharmaceutical ingredients, such as an HDAC6 or EZH2 inhibitor and a pharmaceutical excipient suitable for injection.

The forms in which the compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid and thimerosal.

Sterile injectable solutions are prepared by incorporating an active pharmaceutical ingredient or combination of active pharmaceutical ingredients in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical Delivery

In some embodiments, the invention provides a pharmaceutical composition for transdermal delivery containing an active pharmaceutical ingredient or combination of active pharmaceutical ingredients, such as the HDAC6 or EZH2 inhibitors described herein, and a pharmaceutical excipient suitable for transdermal delivery.

Compositions of the present invention can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of an active pharmaceutical ingredient or combination of active pharmaceutical ingredients in controlled amounts, either with or without another active pharmaceutical ingredient.

The construction and use of transdermal patches for the delivery of pharmaceutical agents is well known in the art. See, e.g., U.S. Pat. Nos. 5,023,252; 4,992,445 and 5,001,139. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra and the HDAC6 or EZH2 inhibitors described herein. The compositions are administered by the oral or nasal respiratory route for local or systemic effect. Compositions in pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, orally or nasally, from devices that deliver the formulation in an appropriate manner. Dry powder inhalers may also be used to provide inhaled delivery of the compositions.

### Other Pharmaceutical Compositions

Pharmaceutical compositions of the HDAC6 or EZH2 inhibitors described herein may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson et al. eds., Handbook of Clinical Drug Data, Tenth Edition, McGraw-Hill, 2002; and Pratt and Taylor, eds., Principles of Drug Action, Third Edition, Churchill Livingston, 1990, each of which is incorporated by reference herein in its entirety.

Administration of an active pharmaceutical ingredient or combination of active pharmaceutical ingredients or a pharmaceutical composition thereof can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), via local delivery by catheter or stent or through inhalation. The active pharmaceutical ingredient or combination of active pharmaceutical ingredients can also be administered intrathecally.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g., PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. The active pharmaceutical ingredient or combination of active pharmaceutical ingredients may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. The active pharmaceutical ingredient or combination of active pharmaceutical ingredients may additionally be administered intravascularly from a balloon used during angioplasty. Extravascular administration of an active pharmaceutical ingredient or combination of active pharmaceutical ingredients via the pericard or via advential application of formulations of the invention may also be performed to decrease restenosis.

Exemplary parenteral administration forms include solutions or suspensions of active compound in sterile aqueous solutions, for example, aqueous propylene glycol or dextrose solutions. Such dosage forms can be suitably buffered, if desired.

The invention also provides kits. The kits include an active pharmaceutical ingredient or combination of active pharmaceutical ingredients, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In selected embodiments, an active pharmaceutical ingredient or combination of active pharmaceutical ingredients are provided as separate compositions in separate containers within the kit. In selected embodiments, an active pharmaceutical ingredient or combination of active pharmaceutical ingredients are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer.

In some embodiments, the invention provides a kit comprising a composition comprising a therapeutically effective amount of an active pharmaceutical ingredient or combination of active pharmaceutical ingredients or a pharmaceutically acceptable salt, solvate, hydrate, cocrystal, or prodrug thereof. These compositions are typically pharmaceutical compositions. The kit is for co-administration of the active pharmaceutical ingredient or combination of active pharmaceutical ingredients, either simultaneously or separately.

In some embodiments, the invention provides a kit comprising (1) a composition comprising a therapeutically effective amount of an active pharmaceutical ingredient or combination of active pharmaceutical ingredients or a pharmaceutically acceptable salt, solvate, hydrate, cocrystal, or prodrug thereof, and (2) a diagnostic test for determining whether a patient's cancer is a particular subtype of a cancer. Any of the foregoing diagnostic methods may be utilized in the kit.

The kits described above are for use in the treatment of the diseases and conditions described herein. In an embodiment, the kits are for use in the treatment of cancer. In some embodiments, the kits are for use in treating solid tumor cancers.

In an embodiment, the kits of the present invention are for use in the treatment of cancer. In an embodiment, the kits of the present invention are for use in the treatment of a cancer selected from the group consisting of bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, aquired immune deficiency syndrome (AIDS)-related cancers (e.g., lymphoma and Kaposi's sarcoma), viral-induced cancer, glioblastoma, esophogeal tumors, hematological neoplasms, non-small-cell lung cancer, chronic myelocytic leukemia, diffuse large B-cell lymphoma, esophagus tumor, follicle center lymphoma, head and neck tumor, hepatitis C virus infection, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

### Dosages and Dosing Regimens

The amounts of the pharmaceutical compositions administered using the methods herein, such as the dosages of HDAC6 or EZH2 inhibitors, will be dependent on the human or mammal being treated, the severity of the disorder or condition, the rate of administration, the disposition of the active pharmaceutical ingredients and the discretion of the prescribing physician. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of the pharmaceutical compositions and active pharmaceutical ingredients may be provided in units of mg/kg of body mass or in mg/m² of body surface area.

In some embodiments, the invention includes a methods of treating a cancer in a human subject suffering from the cancer in which ARID1A is mutated, the method comprising the steps of administering a therapeutically effective dose of an HDAC6 inhibitor and a therapeutically effective dose of an EZH2 inhibitor to the human subject. In some embodiments, the EZH2 inhibitor is administered before the HDAC6 inhibitor. In some embodiments, the HDAC6 inhibitor is administered concurrently with the EZH2 inhibitor. In some embodiments, the EZH2 inhibitor is administered after the HDAC6 inhibitor.

In some embodiments, a pharmaceutical composition or active pharmaceutical ingredient is administered in a single dose. Such administration may be by injection, e.g., intravenous injection, in order to introduce the active pharmaceutical ingredient quickly. However, other routes, including the oral route, may be used as appropriate. A single dose of a pharmaceutical composition may also be used for treatment of an acute condition.

In some embodiments, a pharmaceutical composition or active pharmaceutical ingredient is administered in multiple doses. In an embodiment, a pharmaceutical composition is administered in multiple doses. Dosing may be once, twice, three times, four times, five times, six times, or more than six times per day. Dosing may be once a month, once every two weeks, once a week, or once every other day. In other embodiments, a pharmaceutical composition is administered about once per day to about 6 times per day. In some embodiments, a pharmaceutical composition is administered once daily, while in other embodiments, a pharmaceutical composition is administered twice daily, and in other embodiments a pharmaceutical composition is administered three times daily.

Administration of the active pharmaceutical ingredients in the methods of the invention may continue as long as necessary. In selected embodiments, a pharmaceutical composition is administered for more than 1, 2, 3, 4, 5, 6, 7, 14, or 28 days. In some embodiments, a pharmaceutical composition is administered for less than 28, 14, 7, 6, 5, 4, 3, 2, or 1 day. In some embodiments, a pharmaceutical composition is administered chronically on an ongoing basis—e.g., for the treatment of chronic effects. In some embodiments, the administration of a pharmaceutical composition continues for less than about 7 days. In yet another embodiment the administration continues for more than about 6, 10, 14, 28 days, two months, six months, or one year. In some cases, continuous dosing is achieved and maintained as long as necessary.

In some embodiments, an effective dosage of an active pharmaceutical ingredient disclosed herein is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of an active pharmaceutical ingredient disclosed herein is about 25 mg, about 50 mg, about 75 mg, about 100 mg, about 125 mg, about 150 mg, about 175 mg, about 200 mg, about 225 mg, or about 250 mg.

In some embodiments, an effective dosage of an active pharmaceutical ingredient disclosed herein is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, an effective dosage of an active pharmaceutical ingredient disclosed herein is about 0.35 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg.

In some embodiments, an effective dosage of an active pharmaceutical ingredient disclosed herein is in the range of about 1 mg to about 500 mg, about 10 mg to about 300 mg, about 20 mg to about 250 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg. In some embodiments, an effective dosage of an active pharmaceutical ingredient disclosed herein is about 25 mg, about 50 mg, about 75 mg, about 100 mg, about 125 mg, about 150 mg, about 175 mg, about 200 mg, about 225 mg, or about 250 mg.

In some embodiments, an active pharmaceutical ingredient is adminstered at a dosage of 10 to 200 mg BID, including 50, 60, 70, 80, 90, 100, 150, or 200 mg BID. In some embodiments, an active pharmaceutical ingredient is adminstered at a dosage of 10 to 500 mg BID, including 1, 5, 10, 15, 25, 50, 75, 100, 150, 200, 300, 400, or 500 mg BID.

In some instances, dosage levels below the lower limit of the aforesaid ranges may be more than adequate, while in other cases still larger doses may be employed without causing any harmful side effect—e.g., by dividing such larger doses into several small doses for administration throughout the day.

An effective amount of the combination of the active pharmaceutical ingredient may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1

### Materials and Methods

The materials and methods used in the following examples are described here.

Cell lines and 3D culture conditions. The protocol for using primary cultures of human ovarian clear cell tumour cells was approved by the University of British Columbia Institutional Review Board. The primary tumour cells were cultured in RPMI 1640 supplemented with 10% fetal bovine serum (FBS) and 1% penicillin/streptomycin. Ovarian clear cell carcinoma cell lines (TOV21G, OVTOKO, OVISE and RMG1) were purchased from JCRB (Japanese Collection of Research Bioresources) Cell Bank. TOV21G, OVTOKO, and OVISE cells were cultured in RPMI 1640 supplemented with 10% fetal bovine serum (FBS) and 1% penicillin/streptomycin. RMG1 cells were cultured in 1:1 Dulbecco's modified Eagle's medium (DMEM):F12 supplemented with 10% FBS. Viral packaging cells were cultured in DMEM supplemented with 10% FBS at 37° C. supplied with 5% CO₂. Cells lines are authenticated at The Wistar Institute's Genomics Facility using short tandem repeat DNA profiling. Regular Mycoplasma testing was performed using LookOut Mycoplasma PCR detection (Sigma-Aldrich Co.). 3D membrane cultures were adapted from previously published methods described in Debnath et al. Methods 2003, 30, 256-268, using growth factor reduced-Matrigel (GFR-Matrigel; BD Biosciences). Briefly, a single cell suspension was plated in 8-well chambers covered with Matrigel. Matrigel media with either vehicle control (DMSO) or drug was changed every 4 days and cells were grown for 12 days. Each of the experiments was performed in duplicate in three independent experimental repeats.

Rocilinostat (ACY-1215), Q-VD-Oph, and CAY10603 were obtained from Selleckchem (Selleck Chemicals). The following antibodies were obtained from the indicated suppliers: acetylated-p53 K120 (Abcam), acetylated-p53 K373, acetylated-p53 K382 (Abcam), ARID1A (Santa Cruz), P53, GAPDH (Millipore), Cleaved PARP p85 (Promega), Ki67 (Cell Signaling), cleaved caspase 3 (Cell Signaling), HDAC6 (Cell Signaling and Santa Cruz). Growth factor reduced Matrigel was purchased from Corning.

For immunoblotting, protein was isolated as described in Bitler et al. Nat. Med. 2015, 21, 231-238. Briefly, protein was extracted with RIPA buffer (150 mM NaCl, 1% NP40, 0.5% sodium deoxycholate, 0.1% SDS, 50 mM Tris pH 8.0, and 1 mM PMSF). Protein was separated on a SDS-PAGE and transferred to PVDF membrane. For immunoblot of p53 post-translation modifications, cells were treated with a proteasome inhibitor MG132 (10 μM) to stabilize p53 protein.

For HDAC6 promoter reporter assay, human HDAC6 gene promoter (genomic position: chrX:48658920-48660419) was cloned into pGL2 basic reporter plasmid with firefly luciferase activity (Promega). pGL2-HDAC6 promoter was transfected into RMG1 cells expressing shARID1A or controls. pRL-SV40 reporter plasmid with Renilla luciferase activity (Promega) was used to normalize the transfection efficiency. The firefly and Renilla luciferase activity was measured by Dual-Luciferase Reporter Assay Kit (Promega) 24-hour post-transfection.

Generation of ARID1A CRISPR OVCA429 cells. OVCA429 cells were transfected with CRISPR-ARID1A (pSpCas9(BB)-2A-Puro (PX459)). The ARID1A gRNA sequence is: 5′-CGGGTTGCCCAGGCTGCTGGCGG-3′ (SEQ ID NO:33). Fugene6 transfection reagent (Promega) was used as per manufacturer's specifications. Clonal populations for the loss of ARID1A expression were screened through immunoblot.

Retrovirus production and transduction were performed as described in Aird et al. Cell Reports 2013, 3, 1252-1265, and Bitler et al. Nat. Med. 2015, 21, 231-238. Phoenix cells were used to package the viruses. Lentivirus was packaged using the Virapower Kit from Life Technologies (Carlsbad, Calif.) following the manufacturer's instructions, and as described Aird et al. Cell Reports 2013, 3, 1252-1265, and Bitler et al. Nat. Med. 2015, 21, 231-238. pLKO.1-shARID1As (TRCN0000059090), pLKO.1-shp53 (TRCN0000010814 and TRCN0000003755), pLKO.1-shHDAC6 (TRCN0000004839 and TRCN0000004841), pLKO.1-shCaspase 3 (TRCN0000003550), shCaspase 9 (TRCN0000003583), shCaspase 8 (TRCN0000003577 and TRCN0000003579)and shBRG1 (TRCN0000015549 and TRCN0000015552) were obtained from Open Biosystems. A shRNA to luciferase was used as a control. HDAC6 wildtype and a catalytically inactive H216/611A mutant 21 were obtained from Addgene (Cat. No. 30482 and 30483) and subcloned into lentivirus plasmid pLVX-Puro (Promega) by Xbal and Agel sites using standard molecular cloning protocols. pLKO.1-shARID1As (TRCN0000059089 and TRCN0000059090), pLKO.1-shp53 (TRCN0000010814 and TRCN0000003755), pLKO.1-shHDAC6 (TRCN0000004839 and TRCN0000004841) were obtained from the Molecular Screening Facility at The Wistar Institute. Cells infected with viruses encoding the puromycin resistance gene were selected in 1 μg/ml puromycin.

Reverse-transcriptase quantitative PCR (RT-qPCR). RNA was isolated from cells with RNeasy Mini Kit followed by on-column DNAse digest (Qiagen). mRNA expression for HDAC1-11, ARID1A, and TP53 was determined using SYBR green 1-step iScript (Bio-Rad) with an Life Technologies QuantStudio 3. β-2-microglobulin (B2M) was used as an internal control. Primer sequences are given in Table 1.

TABLE 1
Primer sequences.

| SEQ ID NO | Description | Application | Sequence |
|---|---|---|---|
| 1 | ARID1A forward | qPCR | 5′-TCATGCCCAACCTTCGTATC-3′ |
| 2 | ARID1A reverse | qPCR | 5′-GATGGCTGCTGGGAGTATG-3′ |
| 3 | HDAC1 forward | qPCR | 5′-AATGAAGCCTCACCGAATCC-3′ |
| 4 | HDAC1 reverse | qPCR | 5′-GCTGTGGTACTTGGTCATCTC-3′ |
| 5 | HDAC2 forward | qPCR | 5′-CATGACCCATAACTTGCTGTTAAA-3′ |
| 6 | HDAC2 reverse | qPCR | 5′-ATCTGGTCTTATTGACCGTAGAAA-3′ |
| 7 | HDAC3 forward | qPCR | 5′-CATAGCCTGGTCCTGCATTA-3′ |
| 8 | HDAC3 reverse | qPCR | 5′-CTCTCTGCAGGAAGTCAATGTA-3′ |
| 9 | HDAC4 forward | qPCR | 5′-CAGAAGCAGCAGATCCAGAG-3′ |
| 10 | HDAC4 reverse | qPCR | 5′-TGCTTCATGGCCAGCAT-3′ |
| 11 | HDAC5 forward | qPCR | 5′-AAGAGCCATGCCCAGTTC-3′ |
| 12 | HDAC5 reverse | qPCR | 5′-CTCCTGCTGCAGTTGCT-3′ |
| 13 | HDAC6 forward | qPCR | 5′-TGGTGTTGGATGAGCAGTTAAA-3′ |
| 14 | HDAC6 reverse | qPCR | 5′-AGGACACGCAGCGATCTA-3′ |
| 15 | HDAC7 forward | qPCR | 5′-GCTTCTCCACAAGGACAAGAG-3′ |
| 16 | HDAC7 reverse | qPCR | 5′-GTTGGGATGGACTGTTCTTTCT-3′ |
| 17 | HDAC8 forward | qPCR | 5′-GCATTCAGTTTCACCTCCAAAG-3′ |
| 18 | HDAC8 reverse | qPCR | 5′-TACACTGTAGTACCGTCCCTT-3′ |
| 19 | HDAC9 forward | qPCR | 5′-AGGACAGACCTCAGGATGAT-3′ |
| 20 | HDAC9 reverse | qPCR | 5′-CTGCTTCTGGATTTGTTGCTG-3′ |
| 21 | HDAC10 forward | qPCR | 5′-GCACAGCCCAGAGTATGTATC-3′ |
| 22 | HDAC10 reverse | qPCR | 5′-GAAGTAGATGGCGTCGAACTG-3′ |
| 23 | HDAC11 forward | qPCR | 5′-GTCTACAACCGCCACATCTAC-3′ |
| 24 | HDAC11 reverse | qPCR | 5′-CTTGATGTTCCTCTCCACCTTATC-3′ |
| 25 | p53 forward-ORF | qPCR | 5′-CCAGATGAAGCTCCCAGAATG-3′ |
| 26 | p53 reverse-ORF | qPCR | 5′-TGGGAAGGGACAGAAGATGA-3′ |
| 27 | p53 forward-3′ UTR | qPCR | 5′-GGAGTAGGACATACCAGCTTAGA-3′ |
| 28 | p53 reverse-3′ UTR | qPCR | 5′-CCTACCTAGAATGTGGCTGATTG-3′ |
| 29 | B2M forward | qPCR | 5′-GGCATTCCTGAAGCTGACA-3′ |
| 30 | B2M reverse | qPCR | 5′-CTTCAATGTCGGATGGATGAAAC-3′ |
| 31 | HDAC6 promoter forward | ChIP | 5′-ACTAAGACTCCTTCCACTCAGA-3′ |
| 32 | HDAC6 promoter reverse | ChIP | 5′-ACTATGGTGGTACACAGGAAA-3′ |

Annexin V and mitochondria membrane potentials were measured as follows. Phosphatidylserine externalization was detected using an Annexin V staining kit (Life Technologies) following the manufacturer's instructions. To measure change in mitochondria membrane potential cells were treated with 200 nM of tetramethylrhodamine, ethyl ester (TMRE; Abcam) for 15 min at 37° C. Annexin V and TMRE-positive cells were detected using the LSR18 and analyzed with the FlowJo software module.

Colony formation assays were performed as follows. Cell lines were infected with lentivirus pLKO.1-shRNAs or pLKO.1-control with puromycin selection marker. Infected cells were selected with 1 μg/mL of puromycin for 72 hours and the selected cells were seeded in 12-well or 24-well plates. Cell medium was changed every three days with appropriate drug doses for 14 days or until control wells became confluent. Colonies were washed twice with PBS and fixed with 10% methanol and 10% acetic acid in distilled water. Fixed colonies were stained with 0.005% crystal violet. Integrated density was measured using NIH ImageJ software.

In vitro biochemical HDAC6 deacetylation assays were performed as follows. Activity assay with human HDAC6 was based on a discontinuous liquid chromatography-mass spectrometry (LC-MS) assay as reported in Hai and Christianson, 2016. Briefly, 0.05 μM of HDAC6 was incubated with 100 μM of substrate Ac-LHSGTAK(ac)SVT-COOH in HEPES buffer [20 mM HEPES (pH 7.5), 100 mM NaCl, 5 mM KCl, 1 mM MgCl₂] for 20 min at room temperature, and the reaction was quenched by the addition of acetonitrile (equal volume to the reaction solution). The deacetylation reaction mixtures were detected by (LC-MS) using a Waters SQD equipped with an Acquity UPLC (Waters, Milford, Mass., USA) and quantified by using the standard curves generated from the mass signals of the corresponding deacetylated synthetic peptide (Ac-LHSGTAKSVT-COOH). The assay was run in the presence of 10 μM of HDAC6 specific inhibitor ACY-1215 as a negative control. All the assays were performed in triplicates. The peptide was custom synthesized by Genscript. The human HDAC6 construct was expressed and purified as described in Hai and Christianson, 2016.

Chromatin immunoprecipitation (ChIP) was performed as described in Bitler et al. Nat. Med. 2015, 21, 231-238. The following antibodies were used to perform ChIP: ARID1A (Santa Cruz) or RNA polymerase II (Santa Cruz). An isotype matched IgG was used as a negative control. ChIP DNA was analyzed by quantitative PCR against the promoter of the human HDAC6 gene. Primer sequences are listed in Table 1. For single site PCR, the primers for position −880 upstream of transcription starting site were used.

Immunofluorescence and immunohistochemical staining was performed as follows. Immunofluorescence was performed after 48 hours as indicated by fixing samples in 4% paraformaldehyde and permeabilizing with 0.5% Triton-X. Samples were incubated with primary antibodies for 2 hours at room temperature, highly cross absorbed secondary antibodies (Invitrogen) for 1 hour at room temperature and mounted with prolong anti-fade reagent (Invitrogen). Immuno-stained cells were imaged using a Leica Confocal microscope. Immunohistochemical staining was performed as described previously on consecutive sections from xenografted tumors dissected from control or rocilinostat (ACY-1215) treated immunocompromised nude female mice. Bitler et al. Nat. Med. 2015, 21, 231-238; Bitler et al. Cancer Research 2011, 71, 6184-6194. Expression of the stained markers was scored using a histologic score (H score) as previously described. McCarty, Jr. et al. Cancer research 1986, 46, 4244s-4248s.

Mitochondria were isolated using the Mitochondria Isolation Kit (Thermo Fisher Scientific). Isolation was performed according to manufacturer's instructions using a “B” dounce homogenizer. Protein was isolated from purified mitochondria as described in immunoblotting section. The cytosolic fraction was collected for immunoblot analysis.

The intrabursal orthotopic xenograft in vivo model was performed as described previously (Bitler et al. Nat. Med. 2015, 21, 231-238; Bitler et al. Cancer Research 2011, 71, 6184-6194). The protocols were approved by the Institutional Animal Care and Use Committee (IACUC). Briefly, 1×10⁶ luciferase-expressing TOV-21G or RMG1 cells were unilaterally injected into the ovarian bursa sac of immunocompromised mice (n=6 per group). Two weeks after injection, tumors were visualized by injecting luciferin (intraperitoneal (i.p.): 4 mg/mice) resuspended in PBS and imaged with an In Vivo Imaging System (IVIS). The mice were then randomized into two groups based on luciferase activity and treated with vehicle control (2% DMSO/30% PEG 300/ddH₂O) or rocilinostat (ACY-1215) (50 mg/kg daily) for three weeks and imaged for luciferase activity. Images were analyzed using Live Imaging 4.0 software. At end of the experiments, tumors were surgically dissected and tumor burden was calculated based on tumor weight. Intraperitoneally disseminated tumor nodules were quantified.

Arid1a−/−/Pik3caH1047R genetic clear cell ovarian tumour mouse model. All experiments were approved by IACUC. Transgenic mice with latent mutations in Arid1a and Pik3ca were generated by crossing Arid1aflox/floxmice (kindly provided by Dr. Wang, U. Michigan and crossed onto a C57BL/6J background for 9 generations) with R26-Pik3caH1047R mice carrying inducible Pik3ca mutations (Jackson Laboratory, Jax #016977). Gao et al, 2008, Proc Natl Acad Sci USA 105, 6656-6661. Administration of intrabursal adeno-Cre, performed as previously reported, Scarlett et al. 2012, J Exp Med 209, 495-506, induce ovarian clear cell carcinoma in ˜45 days, which is similar to a previous report. All mice were maintained in specific pathogen-free barrier facilities. To induce tumorigenesis, 6-10 weeks old Pik3caH1047R/Arid1aflox/flox female mice were intrabursally injected adenovirus-Cre as previously described. Mice were randomized and treated with ACY1215 (50 mg/kg) or vehicle control for 21 days as previously published. Following treatment mice were sacrificed and the reproductive tracts were removed. The changes in volumes of tumours formed on the injected ovary were calculated against the contrary side non-injected ovary from the same mice.

Statistical analysis and reproducibility. Experiments were repeated 3 times unless otherwise stated. The representative images were shown unless otherwise stated. Statistical analysis was performed using GraphPad Prism 5 (GraphPad) for Mac OS. Quantitative data are expressed as mean ±SEM unless otherwise stated. Analysis of variance (ANOVA) with Fisher's Least Significant Difference (LSD) was used to identify significant differences in multiple comparisons. For all statistical analyses, the level of significance was set at 0.05. For correlation studies, Pearson correlation was used for calculating P and r value GraphPad Prism 5 (GraphPad) for Mac OS. Imaging analysis was performed blindly but not randomly. Animal experiments were randomized. There was no exclusion from the experiments.

Data availability. Gene expression profiling based on RNA-seq was deposited to Gene Expression Ominibus (GEO Accession Number: GSE84405). RNA-seq data for ARID1A wildtype or mutated human ovarian clear cell carcinoma specimens was obtained from European Genome-Phenome Archive (Accession Number: EGAS 00000000075). HDAC6 expression in ARID1A wildtype human ovarian clear cell carcinomas (n=12) and the specimens with frameshift and non-sense ARID1A mutations that are known to correlate with loss of protein expression (n=7) was compared using Mann-Whitney test. Relative cell growth inhibition in clear cell and endometrioid ovarian cancer cell lines with HDAC6 knockdown was determined using Project Achilles synthetic lethality screening analysis (http://www.broadinstitute.org/igp). For correlation between ARID1A and HDAC6 expression, clear cell and endometrioid ovarian cancer cell lines in cancer cell line encyclopedia (GEO Accession Number: GSE36139) and a microarray database for profiling gene expression in laser capture microdissected human clear cell ovarian tumour specimens and ovarian surface epithelial cells were used (GEO Accession Number: GSE29450). ARID1A chromatin immunoprecipitation followed by next generation sequencing (ChIP-seq) and input tracks at the human HDAC6 gene promoter were based on ChIP-seq data (GEO Accession Number: GSE69568).

Quantitative data are expressed as mean ±SEM unless otherwise stated. Analysis of variance (ANOVA) with Fisher's Least Significant Difference (LSD) was used to identify significant differences in multiple comparisons. Spearman's test was used to measure statistical correlation. For all statistical analyses, the level of significance was set at 0.05.

### Example 2

### ARID1A-Inactivated Cells are Sensitive to HDAC6 Inhibition

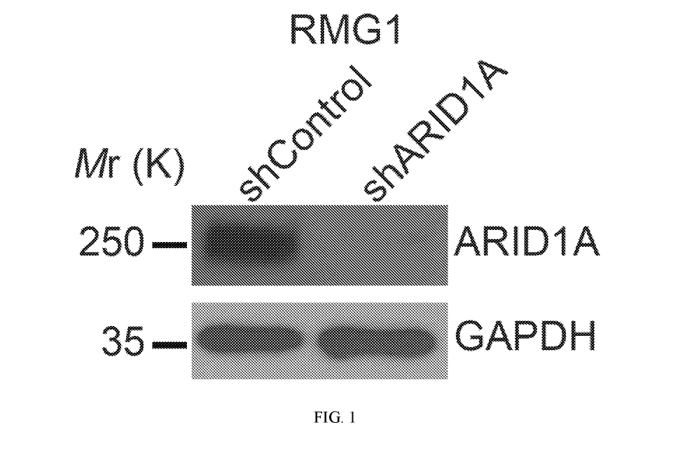

To examine the role of specific HDACs in the context of ARID1A-mutated ovarian cancers, an unbiased short hairpin RNA (shRNA) knockdown-based evaluation against eleven histone deacetylase genes was performed. This was done in the context of ARID1A wild-type ovarian clear cell RMG1 cancer cells with or without ARID1A knockdown (FIG. 1). ARID1A knockdown mimics the loss of ARID1A protein expression caused by >90% of ARID1A mutations in ovarian cancer (Wiegand et al. N. Engl. J. Med. 2010, 363, 1532-1543) and ensure the same genetic background for the unbiased evaluation. Pooled shRNAs were transduced for each of the 11 individual HDACs in ARID1A wild-type RMG1 cells with or without ARID1A knockdown (Table 2).

TABLE 2
shRNAs Used in Knockdown Experiments.

| | | TRCN (RNAi |
|---|---|---|
| shRNA | | Consortium Library No.) |
| shHDAC1 | #1 | TRCN0000004814 |
| | #2 | TRCN0000004815 |
| | #3 | TRCN0000004816 |
| shHDAC2 | #1 | TRCN0000004821 |
| | #2 | TRCN0000004822 |
| | #3 | TRCN0000004823 |
| shHDAC3 | #1 | TRCN0000004826 |
| | #2 | TRCN0000004828 |
| shHDAC4 | #1 | TRCN0000004829 |
| | #2 | TRCN0000004832 |
| | #3 | TRCN0000004833 |
| | #4 | TRCN0000004830 |
| | #5 | TRCN0000004831 |
| shHDAC5 | #1 | TRCN0000004835 |
| | #2 | TRCN0000004837 |
| shHDAC6 | #1 | TRCN0000004839 |
| | #2 | TRCN0000004840 |
| | #3 | TRCN0000004841 |
| shHDAC7 | #1 | TRCN0000004844 |
| | #2 | TRCN0000004847 |
| | #3 | TRCN0000004845 |
| | #4 | TRCN0000004846 |
| shHDAC8 | #1 | TRCN0000004849 |
| | #2 | TRCN0000004852 |
| shHDAC9 | #1 | TRCN0000004854 |
| | #2 | TRCN0000004856 |
| shHDA10 | #1 | TRCN0000004860 |
| | #2 | TRCN0000004862 |
| | #3 | TRCN0000004859 |
| | #4 | TRCN0000004863 |
| shHDAC11 | #1 | TRCN0000017754 |
| | #2 | TRCN0000017756 |
| shARID1A | #1 | TRCN0000059089 |
| | #2 | TRCN0000059090 |
| shP53 | #1 | TRCN0000010814 |
| | #2 | TRCN0000003755 |

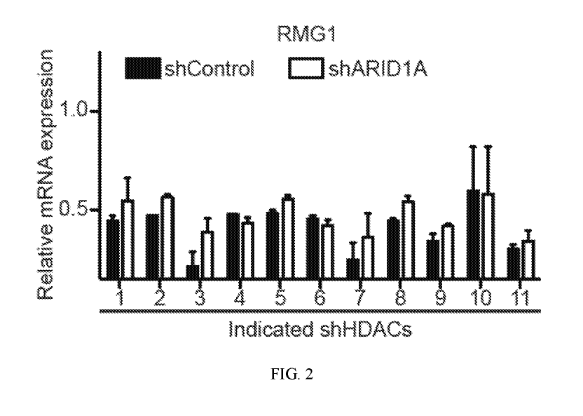

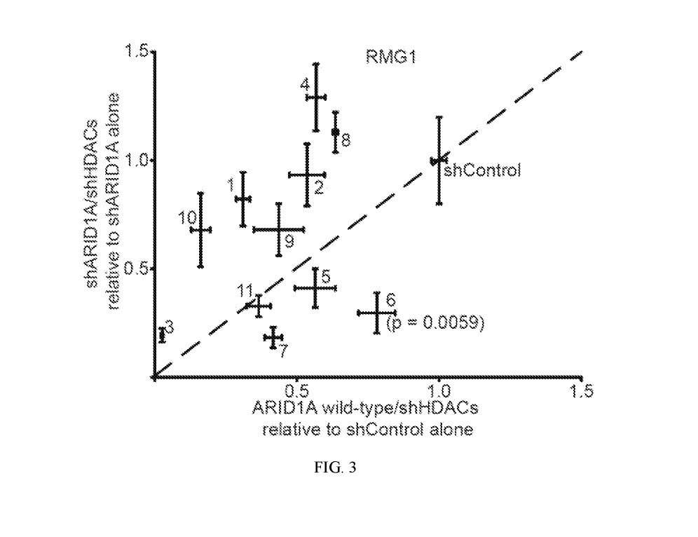

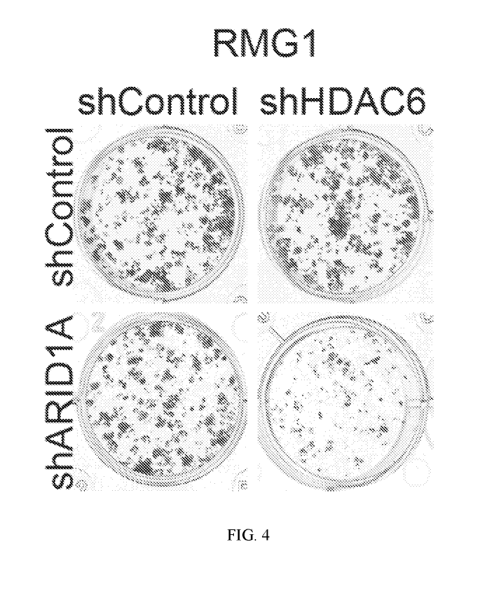

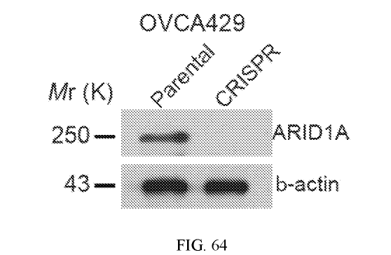

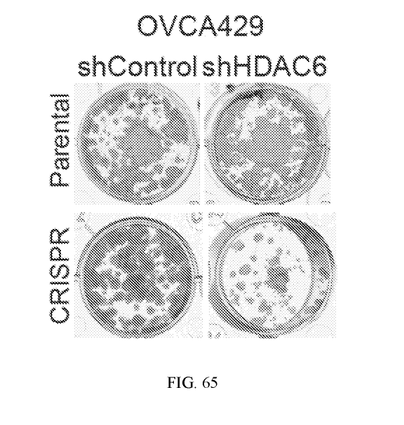

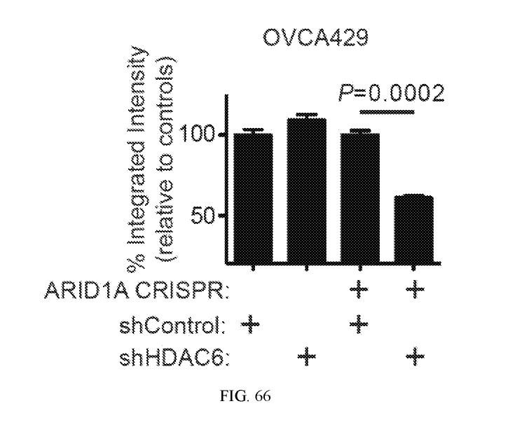

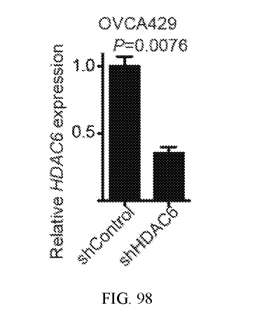

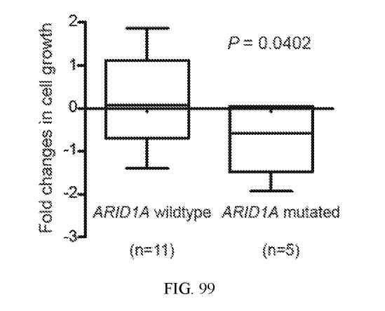

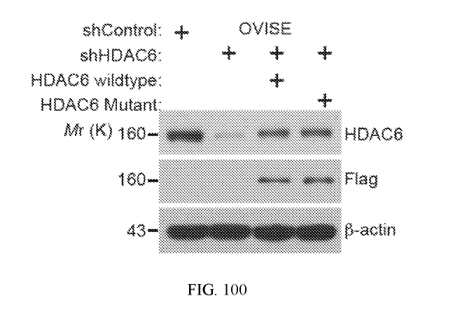

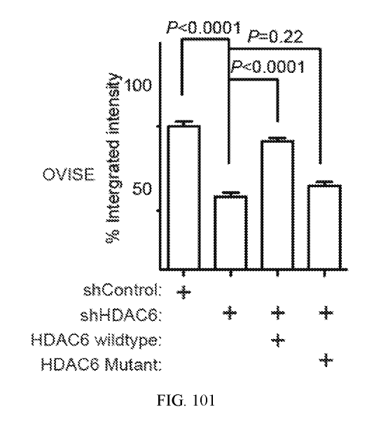

Knockdown of all the HDACs was confirmed by qRT-PCR and a similar degree of knockdown of all the HDACs was found regardless of ARID1A expression (FIG. 2). To measure changes in cell viability, these cells were subjected to a colony formation assay. Similar to previous reports (Bitler et al. Nat. Med. 2015, 21, 231-238), no significant difference was observed between ARID1A wild-type control and ARID1A knockdown cells (FIG. 3, FIG. 4, FIG. 98, and FIG. 99). Significantly, HDAC6 knockdown showed the highest selectivity against ARID1A knockdown cells compared with controls (FIG. 3 and FIG. 4). Likewise, HDAC6 knockdown was selective against ARID1A knockout in ARID1A wildtype OVCA429 cells (FIG. 64, FIG. 65, FIG. 66, and FIG. 98). Consistently, HDAC6 knockdown was selective against ARID1A-mutated ovarian clear cell and endometrioid cancer cell lines in the Project Achilles synthetic lethality screen database (FIG. 99).

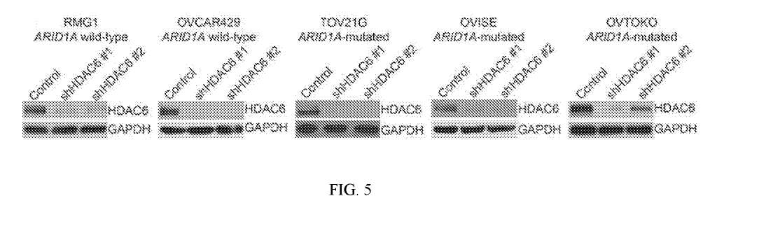

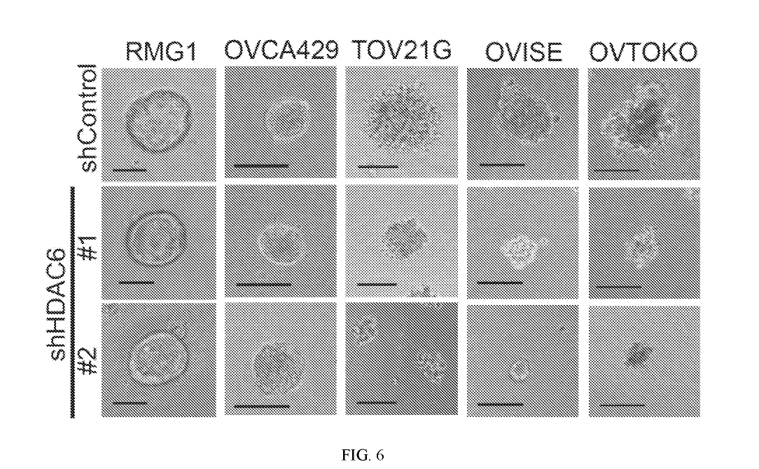

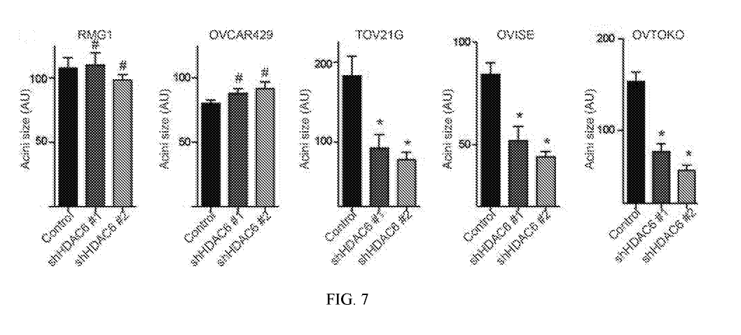

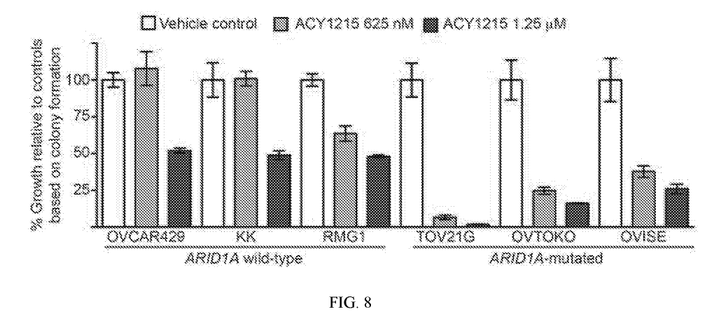

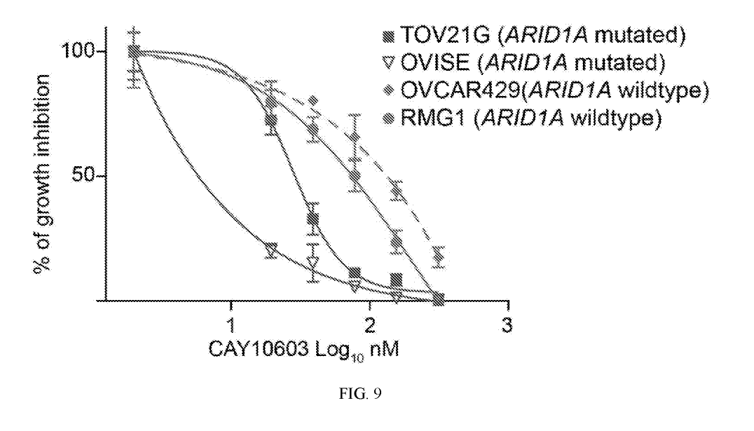

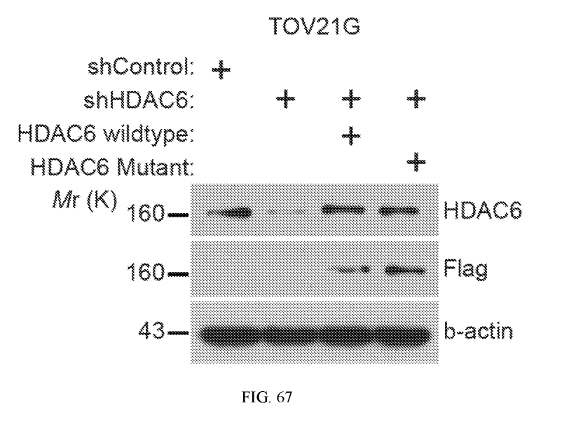

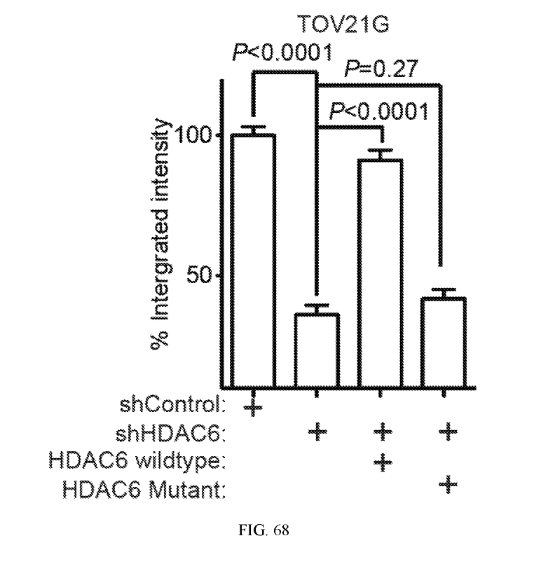

The initial findings were validated in a panel of clear cell ovarian cancer cell lines in 3 dimensional (3D) cultures using Matrigel extracelluar matrix that more closely mimics the tumor microenvironment. HDAC6 knockdown using two independent shRNAs had no appreciable effect on the growth of ARID1A wild-type cells, but significantly suppressed the growth of ARID1A-mutated cells (FIG. 5, FIG. 6, and FIG. 7). The observed growth inhibition depends on the enzymatic activity of HDAC6 because the growth inhibition was rescued by a wildtype HDAC6 but not a catalytically inactive H216/611A mutant (FIG. 2, FIG. 67, FIG. 68, and FIG. 98). Selective and specific HDAC6 deacetylase activity inhibitors have been developed. Thus, two HDAC6 inhibitors were tested, namely rocilinostat (ACY-1215) (Haggarty et al. Proc. Natl. Acad. Sci. USA 2003, 100, 4389-4394; Santo et al. Blood 2012, 119, 2579-2589) and CAY10603 (Kozikowski et al. J. Med. Chem. 2008, 51, 4370-4373), in a panel of cell lines with or without ARID1A mutation. Compared with ARID1A wild-type cells, ARID1A-mutated cells were more sensitive to both of the tested HDAC6 inhibitors (FIG. 8 and FIG. 9). Together, it was concluded that ARID1A-inactivated cells are selectively sensitive to HDAC6 inhibition.

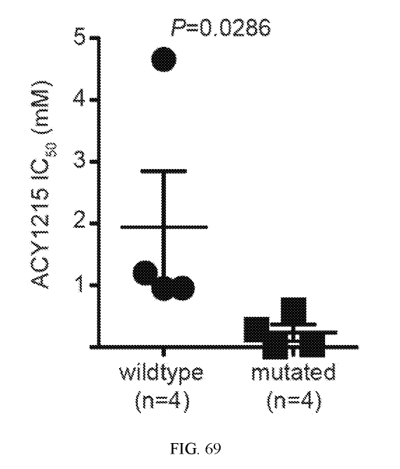

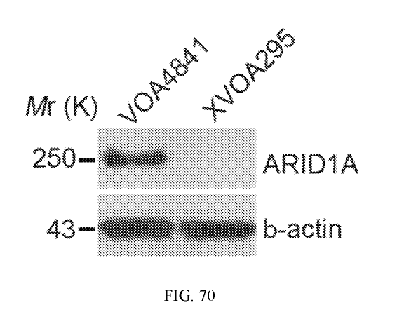

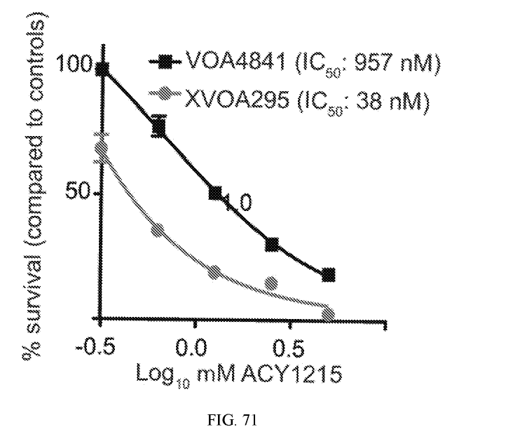

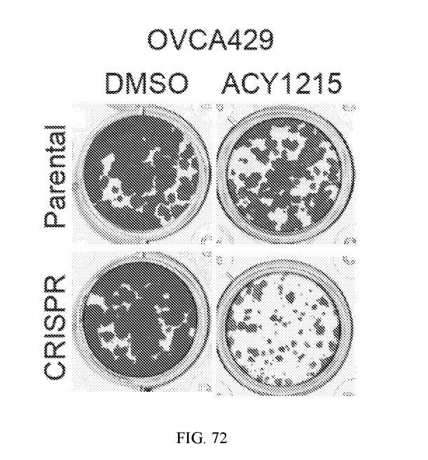

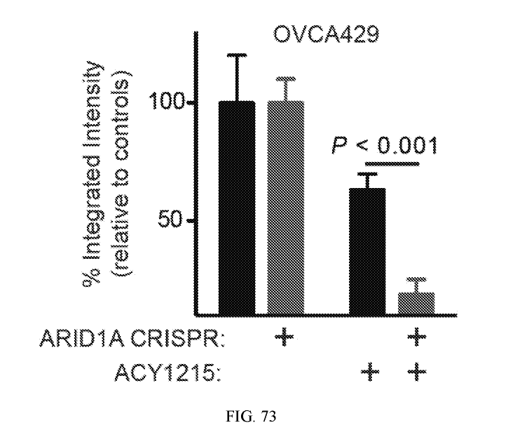

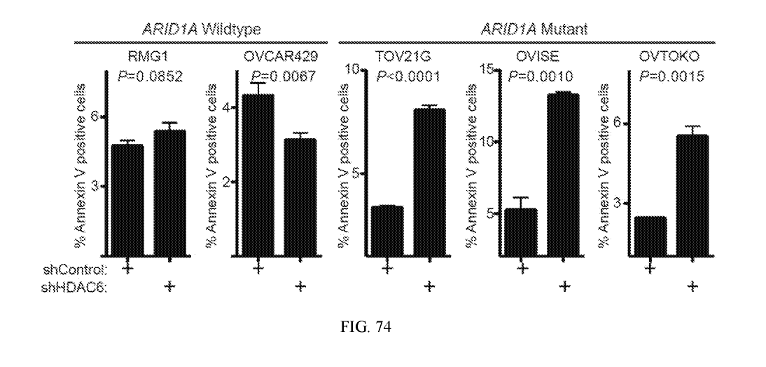

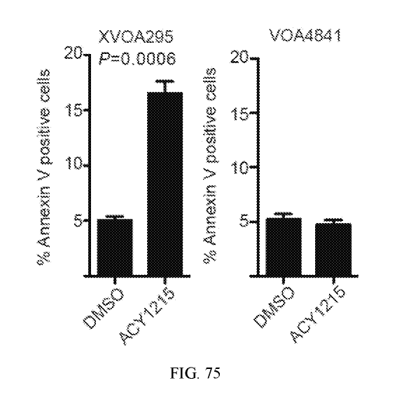

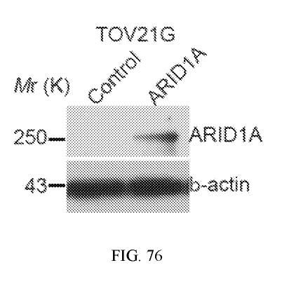

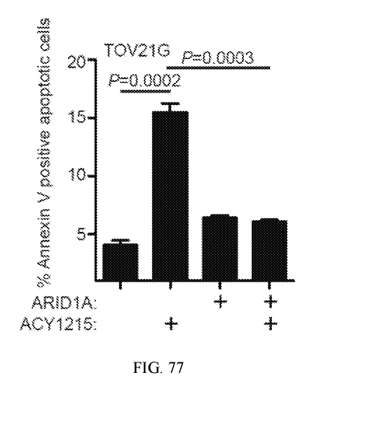

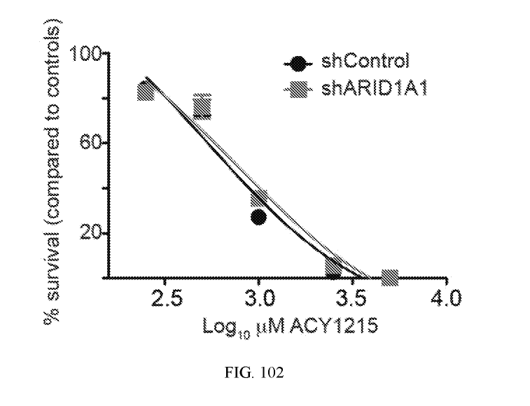

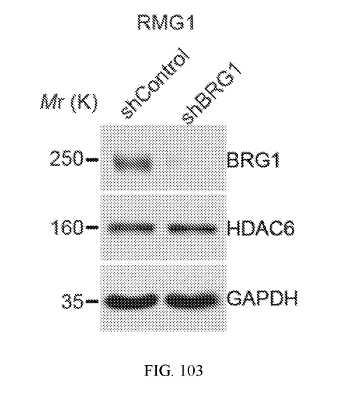

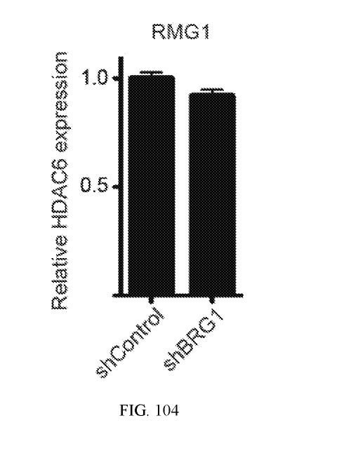

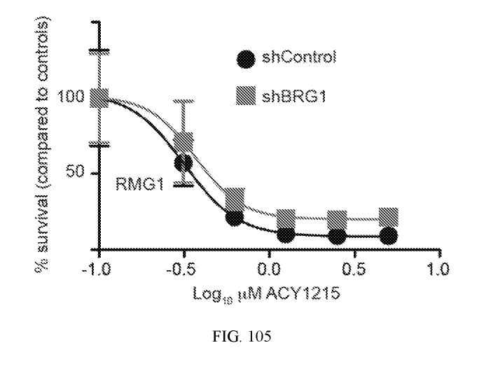

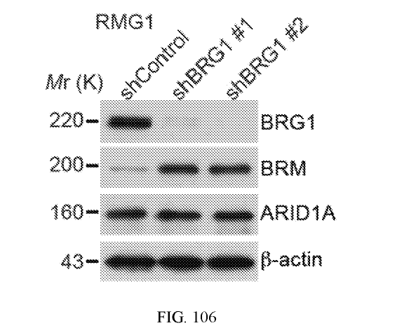

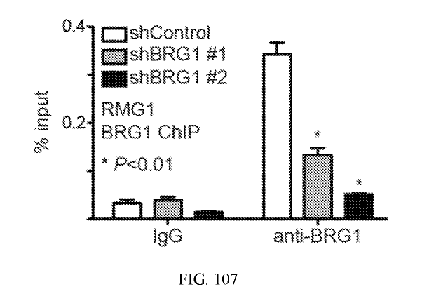

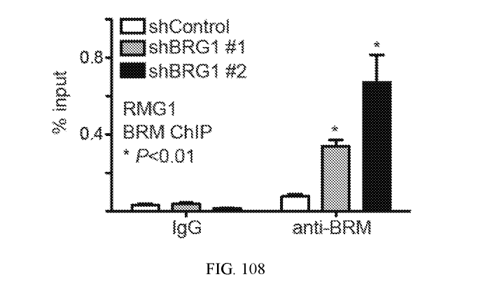

The IC₅₀ values of ACY1215 in primary cells are comparable to those observed in cell lines (FIG. 69, FIG. 70, and FIG. 71). ARID1A knockout significantly increased the sensitivity of ARID1A wildtype OVCA429 cells to ACY1215 (FIG. 72 and FIG. 73). Conversely, restoration of wildtype ARID1A in ARID1A-mutated TOV21G cells reduced the sensitivity of these cells to ACY1215 (FIG. 102, FIG. 103, and FIG. 104). Interestingly, knockdown of other SWI/SNF subunits such as BRG1 1 did not increase ACY1215 sensitivity (FIG. 105, FIG. 106, and FIG. 107). This correlates with a compensation of BRG1 loss by the mutually exclusive catalytic subunit BRM (FIG. 108).

### Example 3

### HDAC6 Inhibition Promotes Apoptosis in ARID1A-Inactivated Cells

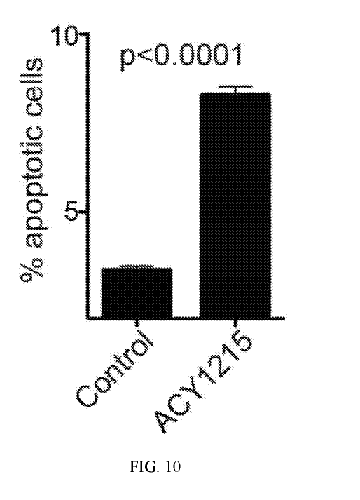

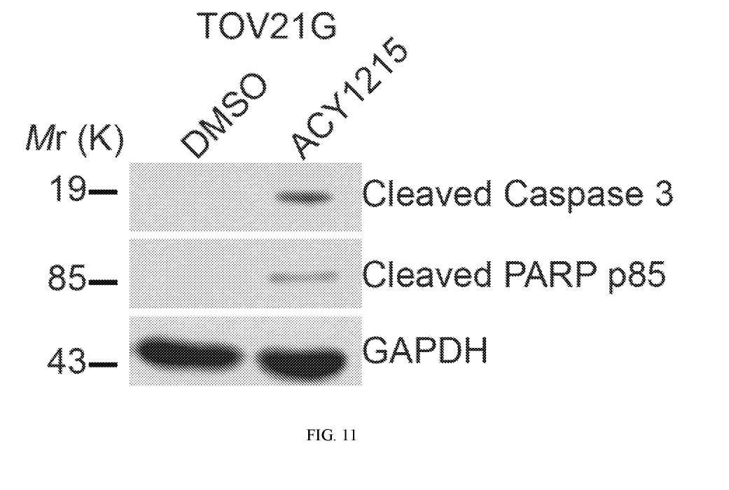

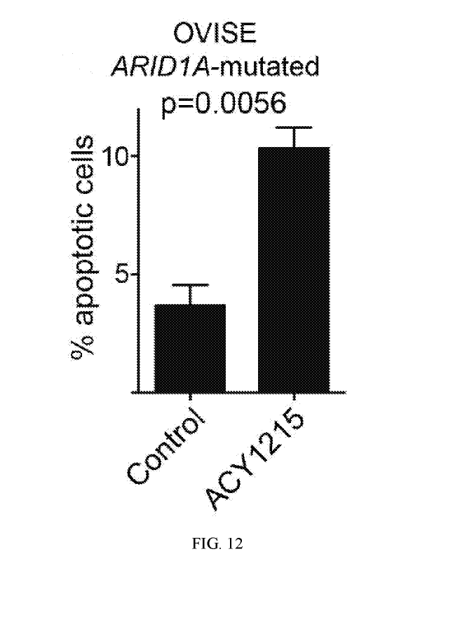

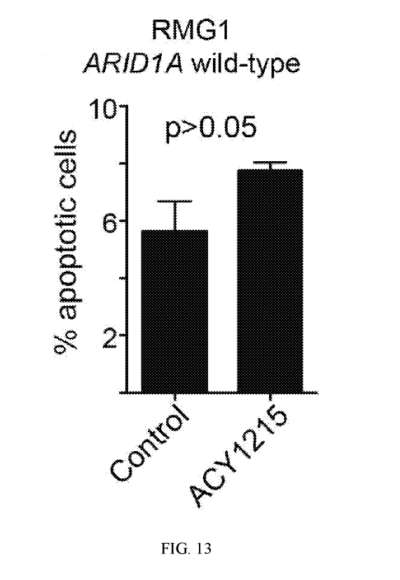

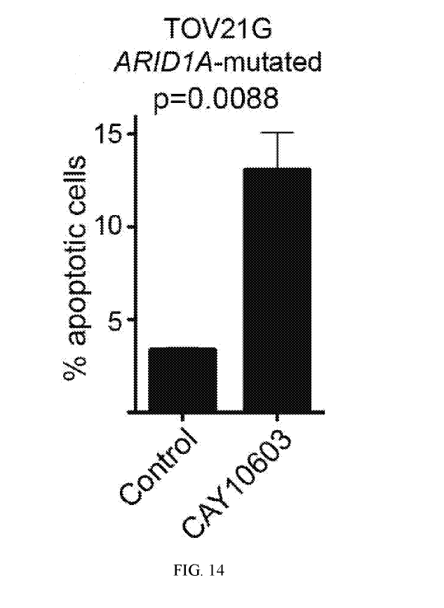

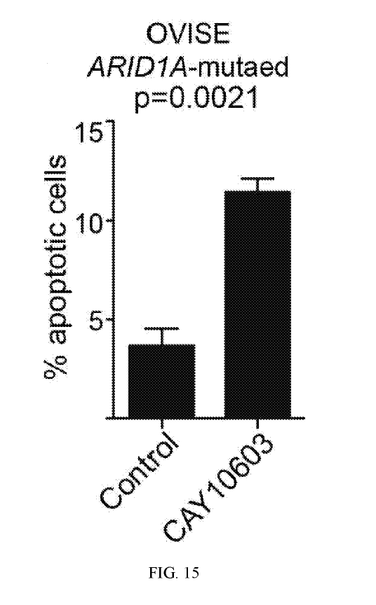

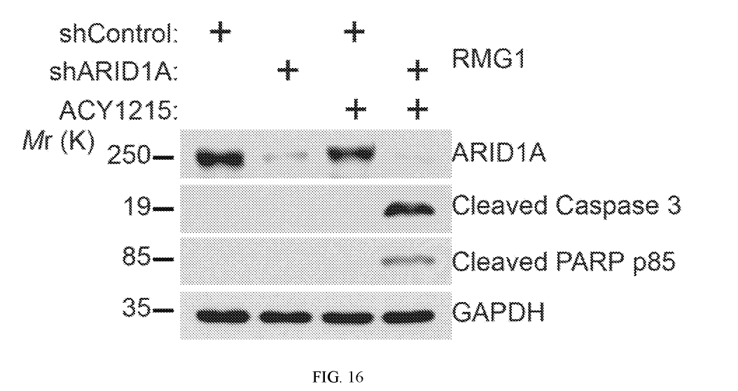

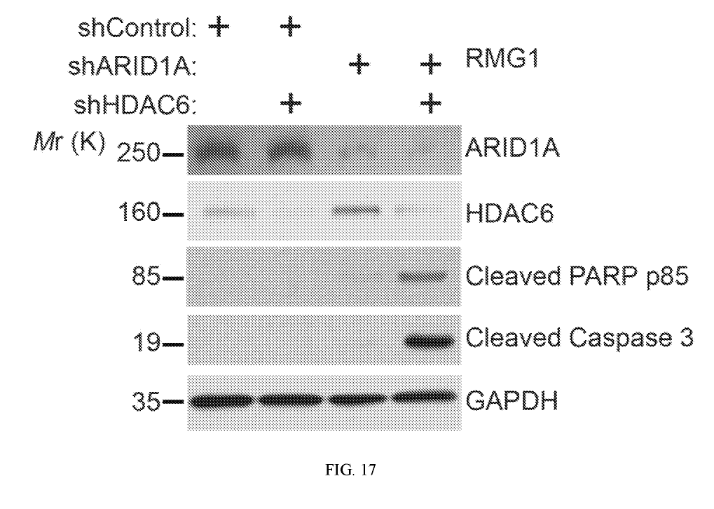

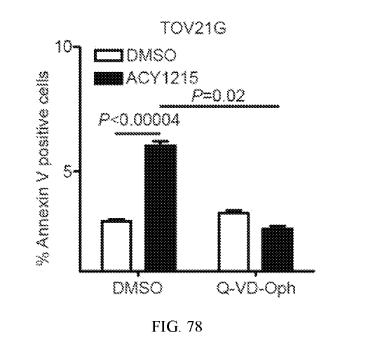

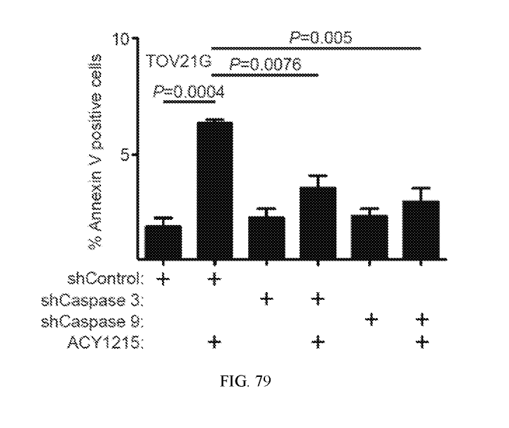

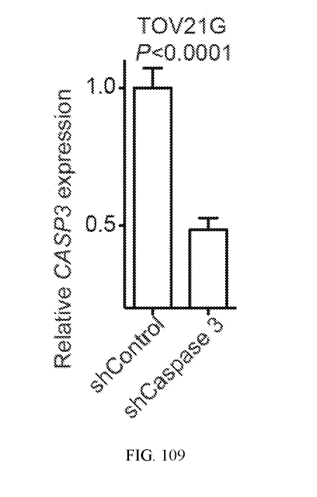

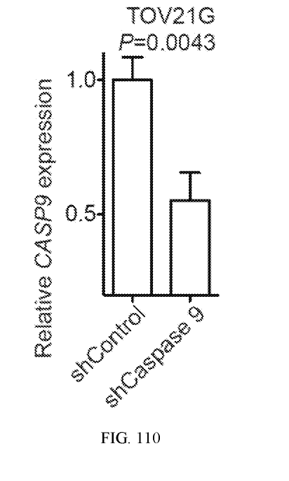

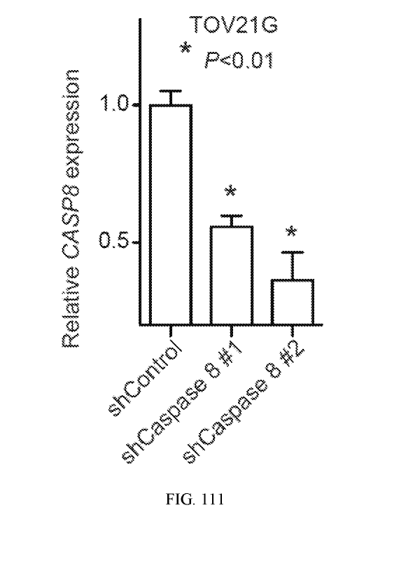

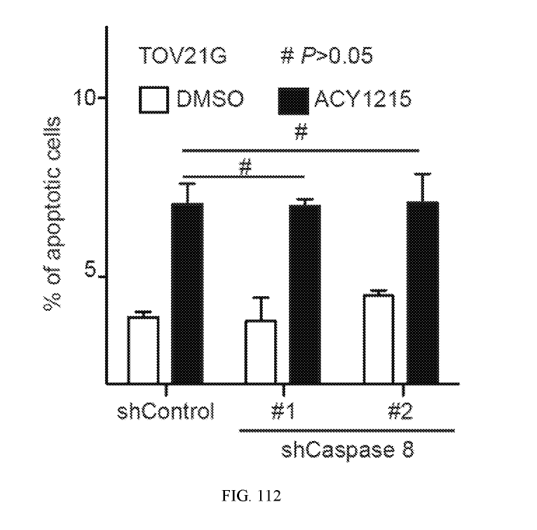

The mechanism whereby the HDAC6 inhibition suppresses the growth of ARID1A-inactivated cells was determined. Notably, HDAC6 inhibitor rocilinostat treatment induced apoptosis of ARID1A-mutated cells as shown by an increase in Annexin V positive cells and upregulation of cleaved caspase 3 and cleaved PARP p85 (FIG. 10, FIG. 11, and FIG. 12). Consistent with the observed selectivity of HDAC6 inhibition against ARID1A inactivation (FIG. 1 to FIG. 8), rocilinostat did not induce a significant increase in apoptosis in ARID1A wild type cells (FIG. 13). Similar results were also obtained using an independent HDAC6 inhibitor CAY10603 (FIG. 14 and FIG. 15). Additionally, HDAC6 inhibition using rocilinostat in ARID1A wild-type cells that were knocked down ARID1A significantly increased markers of apoptosis (FIG. 16). This further supports our finding that ARID1A inactivation conveys sensitivity to HDAC6 inhibition. Similarly apoptosis was also induced by HDAC6 knockdown in ARID1A knockdown cells but not in control ARID1A wild-type cells (FIG. 17). This further supports the notion that the observed apoptosis is due to inhibition of HDAC6 activity. Notably, a pan-caspase inhibitor Q-VD-Oph or knockdown of intrinsic apoptotic pathway initiator caspase 9 or effector caspase 3 significantly suppressed the apoptosis induced by ACY1215 (FIG. 78, FIG. 79, FIG. 109, and FIG. 110). In contrast, knockdown of Caspase 8, the caspase of the extrinsic apoptotic pathway 23, did not affect the apoptosis induced by ACY1215 (FIG. 111 and FIG. 112). Based on these findings, it is shown that HDAC6 inhibition promotes apoptosis in ARID1A-inactivated cells.

### Example 4

### ARID1A Directly Represses HDAC6 Gene Transcription

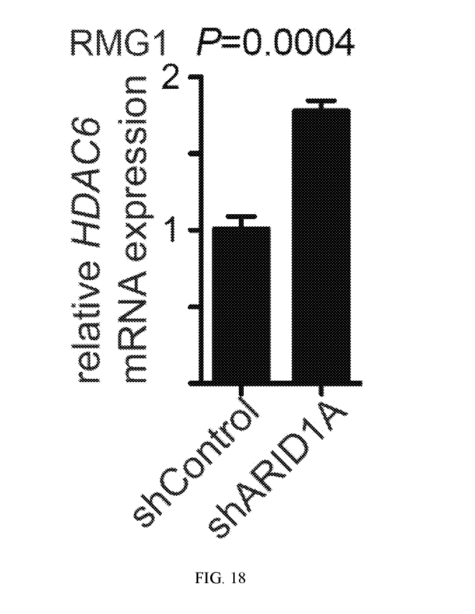

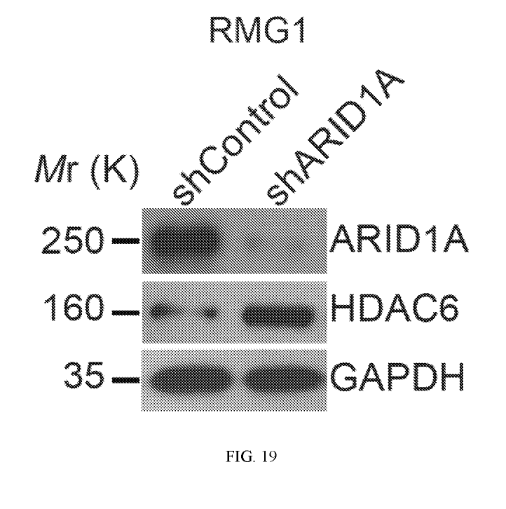

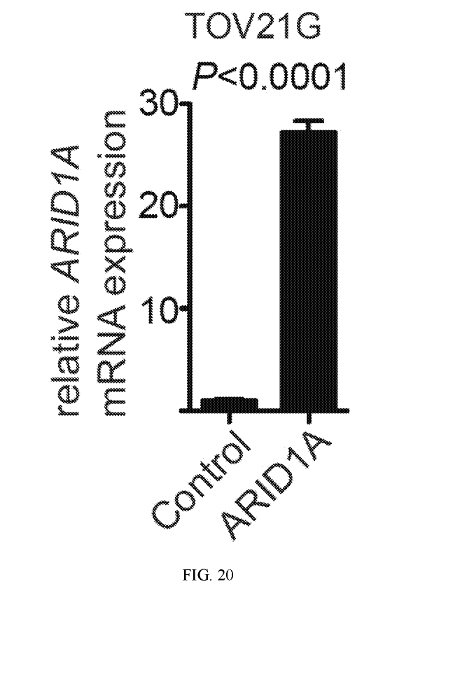

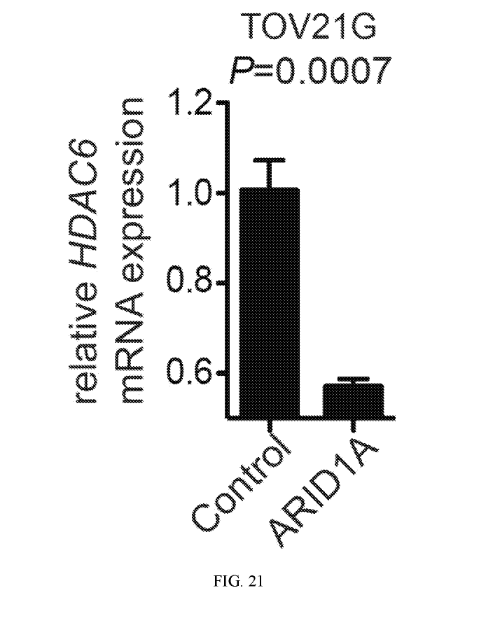

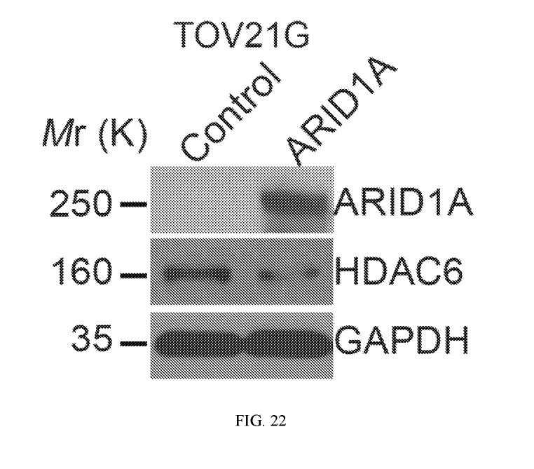

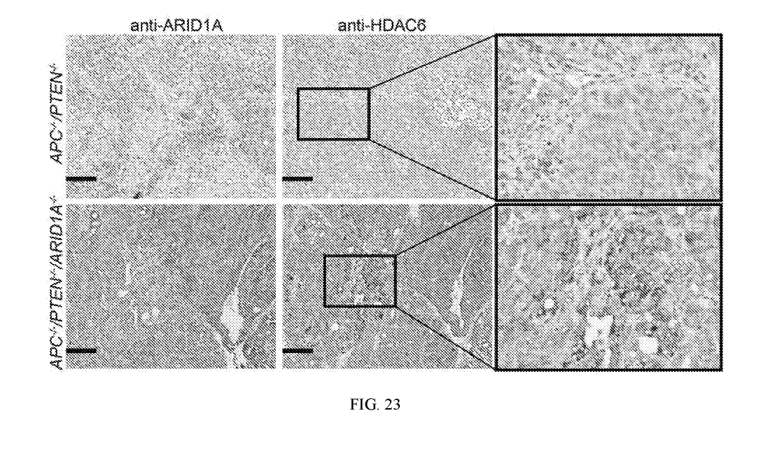

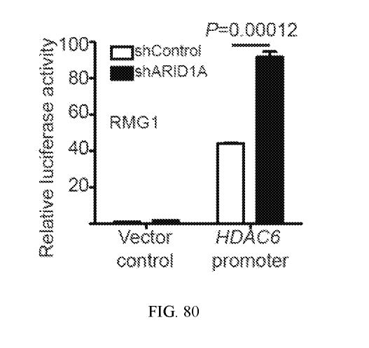

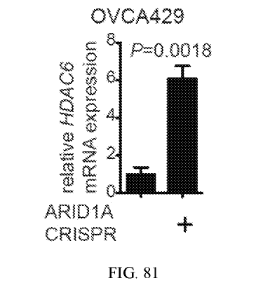

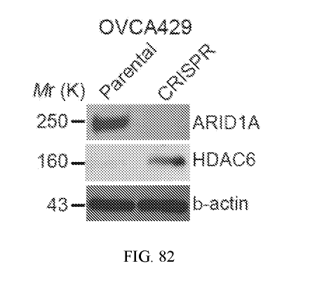

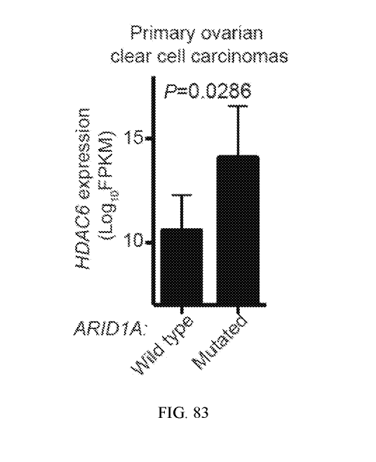

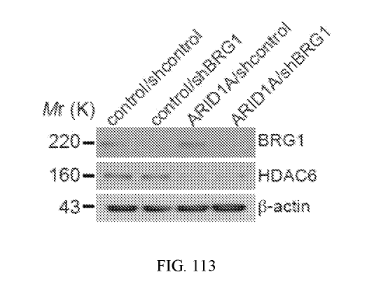

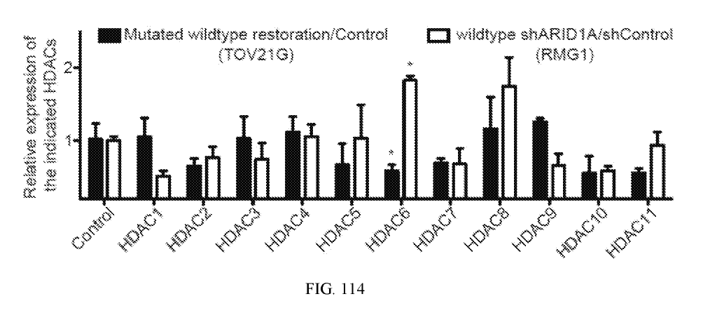

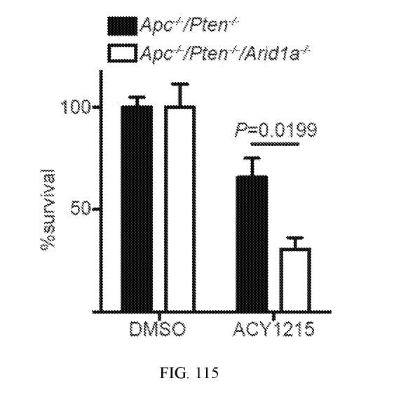

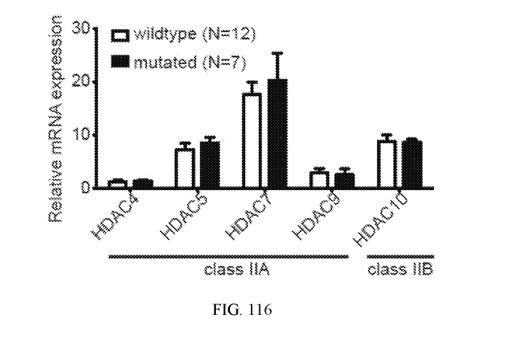

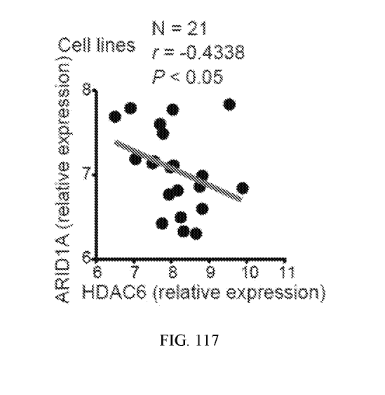

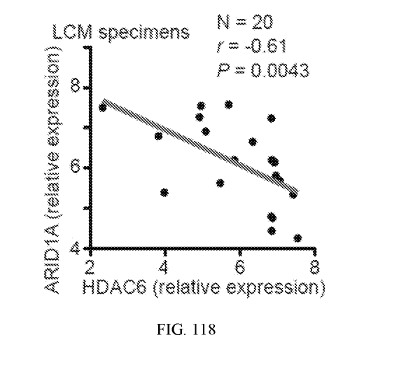

Experiments were performed to determine whether ARID1A affects HDAC6 expression levels. A significant increase in HDAC6 mRNA and protein expression in ARID1A-wildtype cells was observed upon ARID1A knockdown (FIG. 18 and FIG. 19), which correlates with an increase in HDAC6 promoter activity (FIG. 80). Similarly, HDAC6 was expressed at a higher level in ARID1A knockout cells compared with parental ARID1A wildtype cells (FIG. 81 and FIG. 82). Conversely, HDAC6 expression was significantly repressed when wild-type ARID1A expression was restored in ARID1A-mutated cells (FIG. 20 and FIG. 21). BRG1 knockdown did not affect repression of HDAC6 by wildtype ARID1A restoration in ARID1A-mutated cells (FIG. 113), which is consistent with the observation that BRG1 knockdown did not affect HDAC6 expression in ARID1A wildtype cells (FIG. 105 and FIG. 106). Notably, HDAC6 is the only HDAC that is unregulated by ARID1A knockdown in ARID1A wildtype RMG1 cells and downregulated by wildtype ARID1A restoration in ARID1A146 mutated TOV21G cells (FIG. 114). Next, it was determined whether ARID1A regulates HDAC6 in vivo. To do so, the HDAC6 expression in genetic mouse models of ovarian endometrioid carcinomas developed from conditional Apc^{-/-}/Pten^{-/-} and Apc^{-/-}/Pten^{-/-/Arid}1a^{-/-} mice was compared as previously reported. Zhai et al. J. Pathol. 2016, 238, 21-30. These two mouse ovarian endometrioid carcinoma models allowed examination of ARID1A-dependent changes on the same genetic background. The HDAC6 expression was examined by immunohistochemical (IHC) staining. Indeed, compared with ovarian tumors developed from Apc^{-/-}/Pten^{-/-} mice, HDAC6 was expressed at a significantly higher level in tumors developed from Apc^{-/-}/Pten^{-/-}/Arid1a^{-/-} mice (n=5 individual mice/group, p=0.0232) (FIG. 22 and FIG. 23). Consistently, cells derived from Apc−/−/Pten−/−/Arid1a−/− tumours are more sensitive to ACY1215 compared with those derived from Apc−/−/Pten−/− tumours (FIG. 115). HDAC6 was the only class II HDAC that is expressed at significantly higher levels in ARID1A-mutated compared to wildtype primary human clear cell ovarian carcinomas (FIG. 83 abd FIG. 116). In addition, ARID1A expression negatively correlates with HDAC6 expression in both clear cell and endometrioid ovarian cancer cell lines and laser capture microdissected specimens based on database mining (FIG. 117 and FIG. 118). Together, these findings support the notion that ARID1A represses HDAC6 expression and ARID1A inactivation upregulates HDAC6 expression.

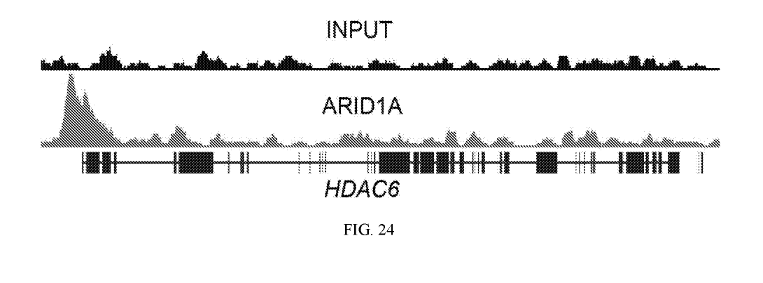

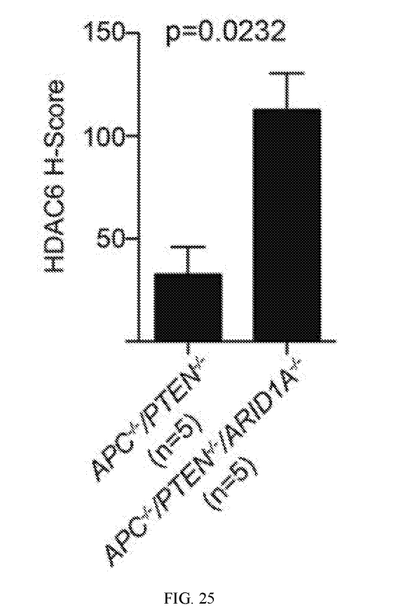

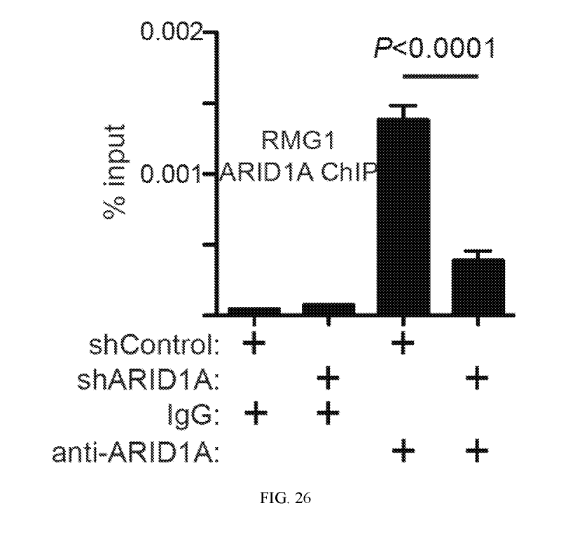

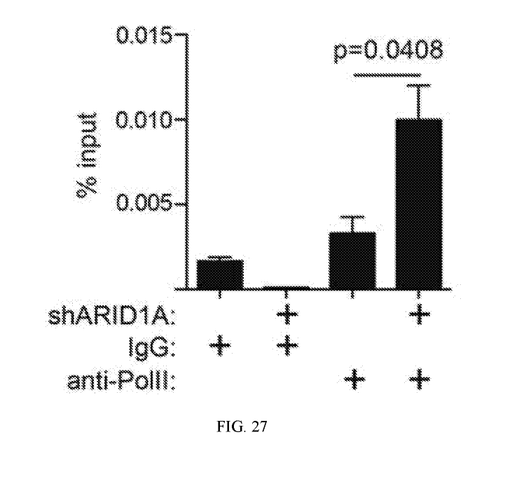

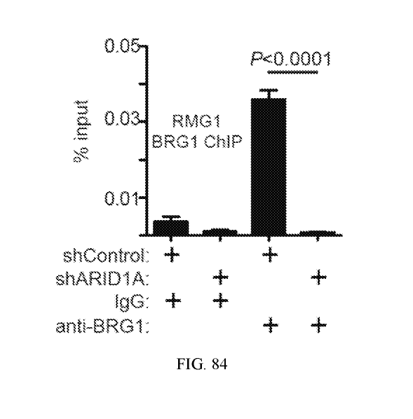

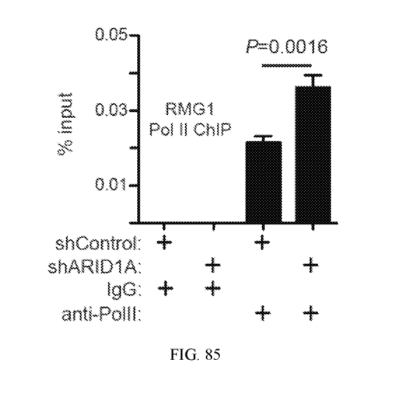

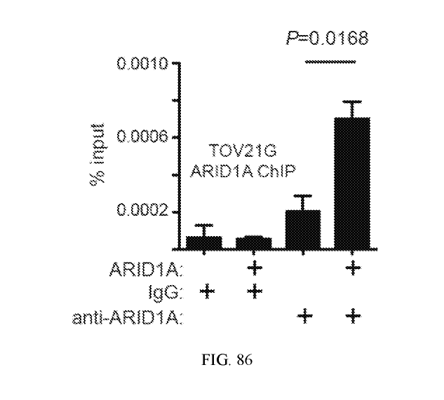

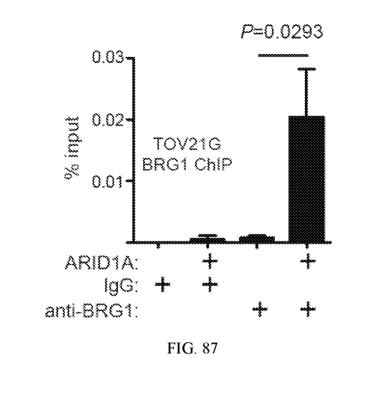

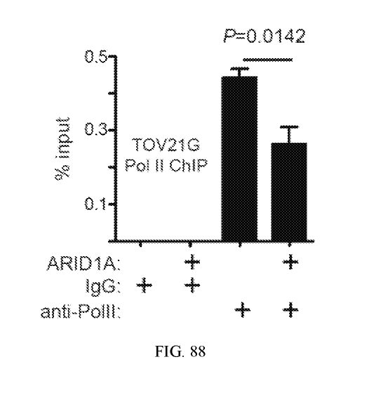

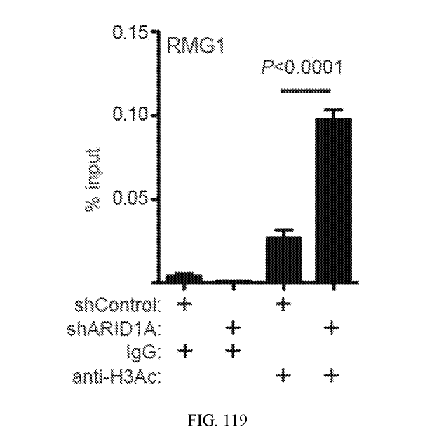

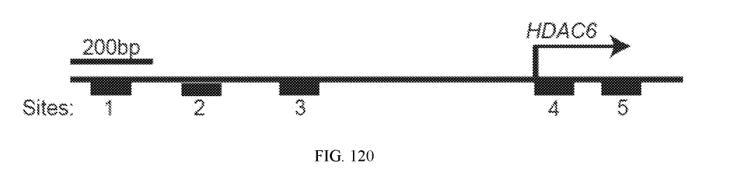

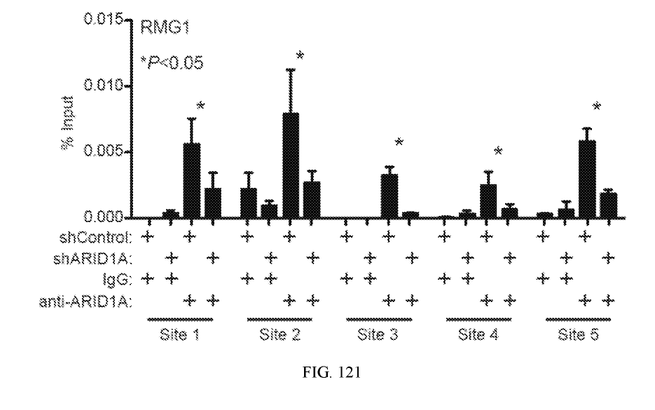

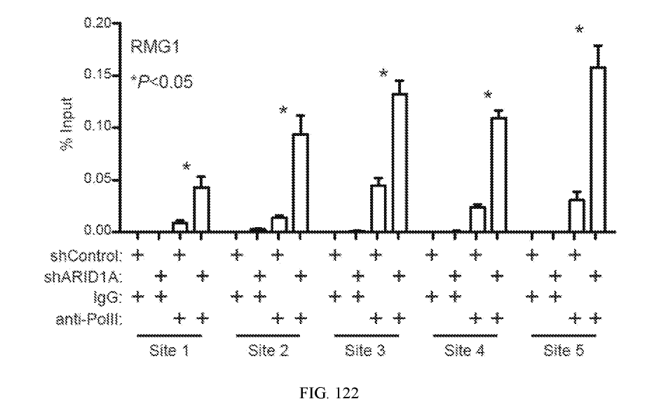

SWI/SNF complexes contribute to both gene activation and repression in a context-dependent manner. Wilson and Roberts, Nat. Rev. Cancer 2011, 11, 481-92. Previous studies showed that ARID1A promotes the expression of tumor suppressors such as PIK3IP1 and CDKN1A. Bitler et al. Nat. Med. 2015, 21, 231-238; Guan et al. Cancer Research 2011, 71, 6718-6727. Thus, it was determined whether ARID1A directly represses HDAC6 expression by mining a published ARID1A ChIP-seq database. Raab et al. PLoS Genet. 2015, 11, e1005748. Indeed, there is a significant enrichment of ARID1A at HDAC6 promoter regions (FIG. 24). Validating these findings, a significant association of ARID1A with the HDAC6 gene promoter in ARID1A wild-type cells was observed (FIG. 25). Supporting the notion that ARID1A directly suppresses HDAC6 transcription, ARID1A knockdown reduced its association with the HDAC6 gene promoter (FIG. 26, FIG. 120, and FIG. 121). This correlated with an increase in RNA polymerase II's association with the HDAC6 gene promoter and upregulation of HDAC6 in these cells (FIG. 27, FIG. 84, FIG. 85, FIG. 119, and FIG. 122). Conversely, wildtype ARID1A restoration in ARID1A-mutated TOV21G cells correlated with an increase in ARID1A and BRG1 and a decrease in Pol II's association with the HDAC6 gene promoter (FIG. 86, FIG. 87, and FIG. 88). Taken together, ARID1A was identified as a direct repressor of HDAC6 gene transcription.

### Example 5

### Lysine 120 Residue Acetylated p53 (p53K120Ac) is a Direct Substrate for HDAC6-Mediated Deacetylation

Next generation sequencing revealed that ARID1A and TP53 mutations are typically mutually exclusive in a number of cancer types in the TCGA database (TABLE 3).

TABLE 3
Analysis of sequencing for ARID1A and TP53 mutations.

| | | Mutation | | | | |
|---|---|---|---|---|---|---|
| | Total | | | ARID1A | | |
| Cancer Type | Cases | ARID1A | TP53 | TP53 | p-value | Reference |
| Uterine corpus endometerioid | 240 | 71 | 68 | 9 | <0.001 | TCGA |
| carcinoma | | | | | | |
| Prostate adenocarcinoma | 332 | 6 | 47 | 0 | <0.001 | TCGA |
| Cervical squamous cell carcinoma | 191 | 15 | 7 | 2 | <0.001 | TCGA |
| Kideny renal clear cell carcinoma | 415 | 12 | 7 | 0 | <0.001 | TCGA |
| Stomach adenocarcinoma | 287 | 98 | 138 | 38 | 0.021 | TCGA |
| Colorectal adenocarcinoma | 220 | 22 | 112 | 7 | 0.023 | TCGA |
| Ovarian clear cell and | 77 | 34 | 6 | 0 | 0.031 | PMCID: |
| endometerioid carcinoma | | | | | | 21900401 |
| Pancreatic cancer | 109 | 16 | 49 | 5 | 0.043 | TCGA |

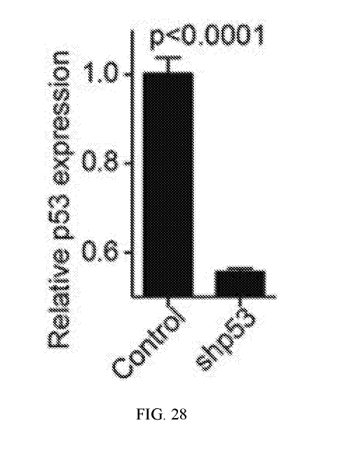

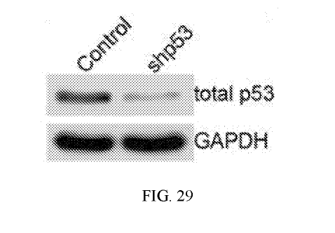

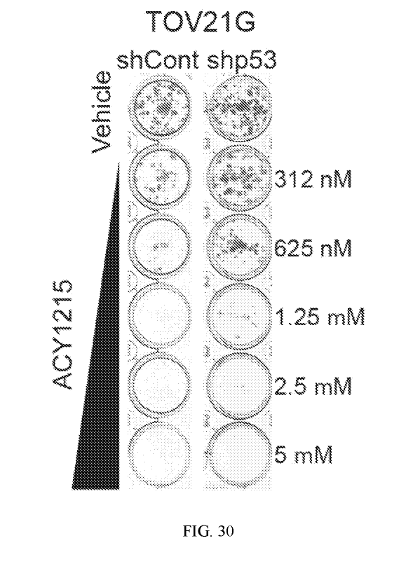

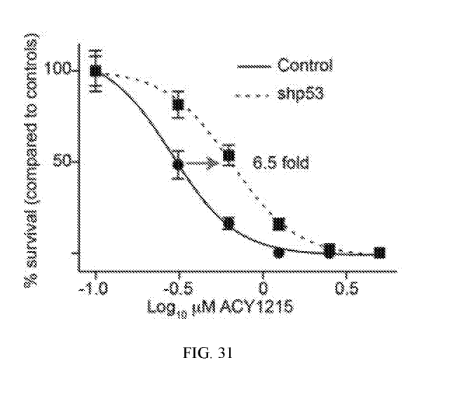

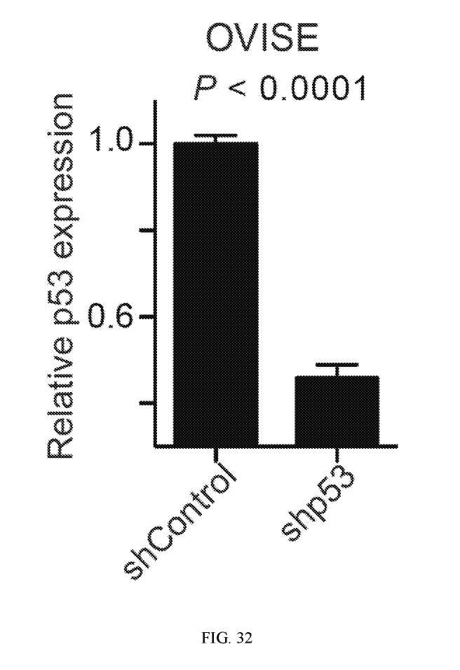

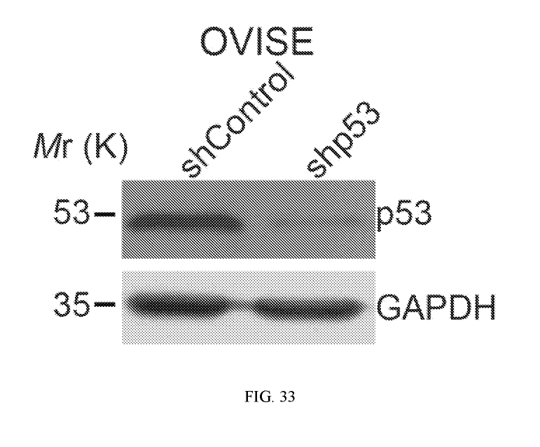

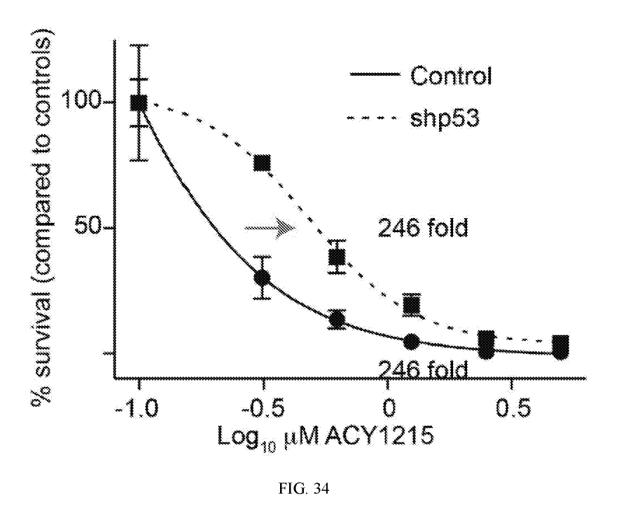

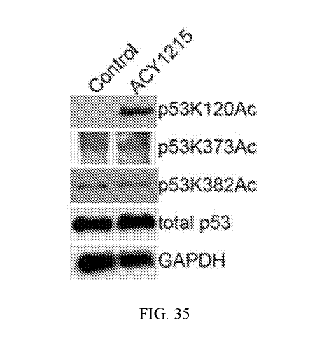

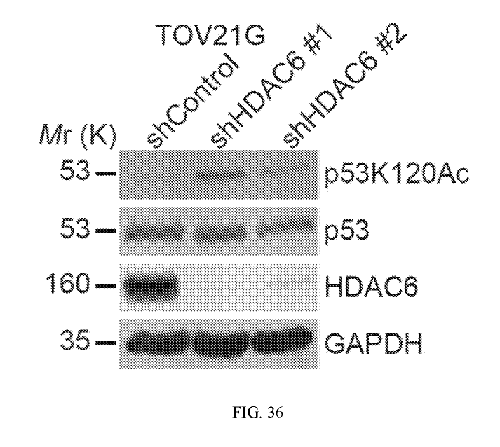

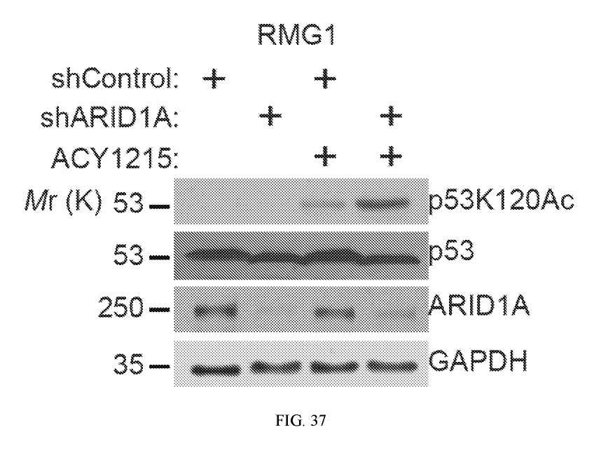

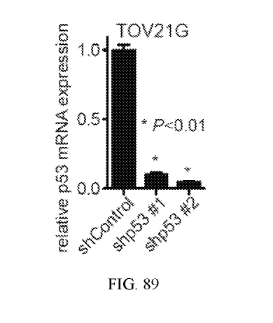

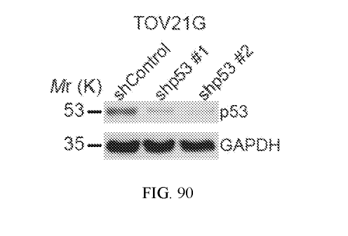

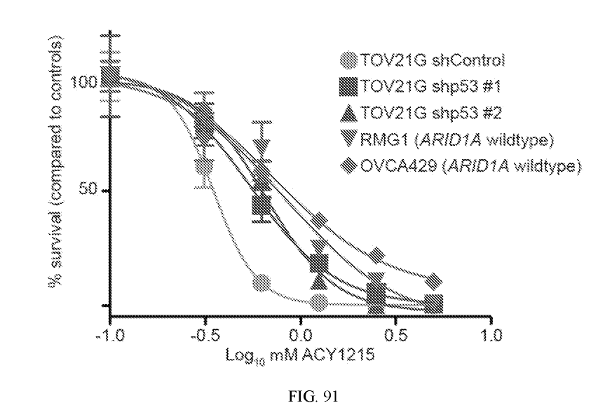

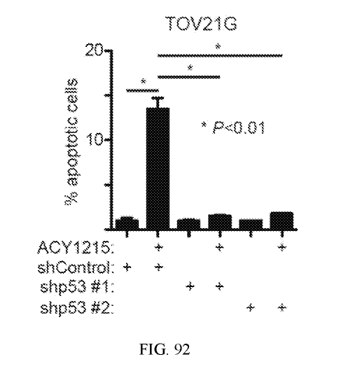

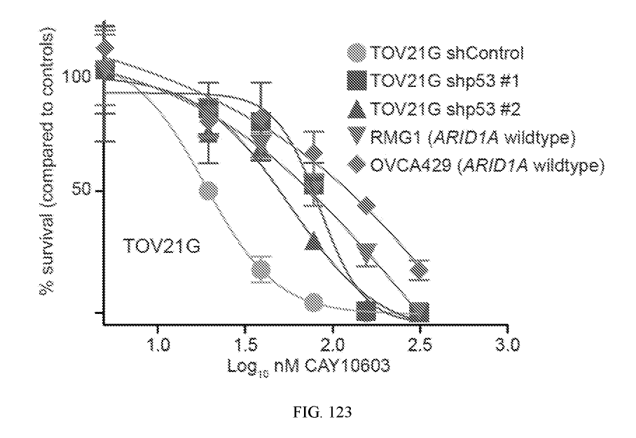

Indeed, ARID1A and TP53 mutations also show a mutually exclusive pattern in ovarian clear cell carcinomas, as described in Guan et al. Cancer Research 2011, 71, 6718-6727. Since HDAC6 inhibition induces apoptosis in ARID1A inactivated cells and p53 is a key regulator of apoptosis, it was determined whether p53 is necessary for the observed growth inhibition and apoptosis. Notably, knockdown of p53 expression significantly impaired the apoptosis and growth inhibition induced by the HDAC6 inhibitor in ARID1A inactivated cells (FIG. 28, FIG. 29, FIG. 30, FIG. 31, FIG. 32, FIG. 33, and FIG. 34). Knockdown of p53 expression significantly impaired the apoptosis and growth inhibition induced by the HDAC6 inhibitor ACY1215 in ARID1A-mutated TOV21G (FIG. 30, FIG. 89, FIG. 90, FIG. 91, and FIG. 92) and OVISE cells (FIG. 32, FIG. 33, and FIG. 34). Similar results were also obtained for another HDAC6 inhibitor CAY10603 (FIG. 123). However, HDAC6 inhibition did not affect p53 expression levels (FIG. 35), indicating that HDAC6 may regulate p53 post-translational modifications. Since HDAC6 is a deacetylase, the changes of p53 acetylation status on the lysine residues of 120, 373, and 382 were evaluated, which are known to regulate apoptosis. These residues were evaluated in ARID1A-mutated cells following treatment with the HDAC6 inhibitor rocilinostat. p53K120Ac was upregulated by rocilinostat treatment, while the acetylation status of lysine 373 or 382 residue was unchanged (FIG. 35). To confirm the increase in p53K120Ac was specific for HDAC6 inhibition, HDAC6 was knocked down with two individual shRNAs. A strong concordance of the level of HDAC6 knockdown and the observed increase of p53K120Ac was observed (FIG. 36). p53K120Ac was also evaluated in ARID1A wild-type with or without ARID1A knockdown after rocilinostat treatment. Indeed, a significant increase in p53K120Ac was observed in the ARID1A knockdown cells compared with controls (FIG. 37). Thus, it was concluded that HDAC6 inhibition increases p53K120Ac.

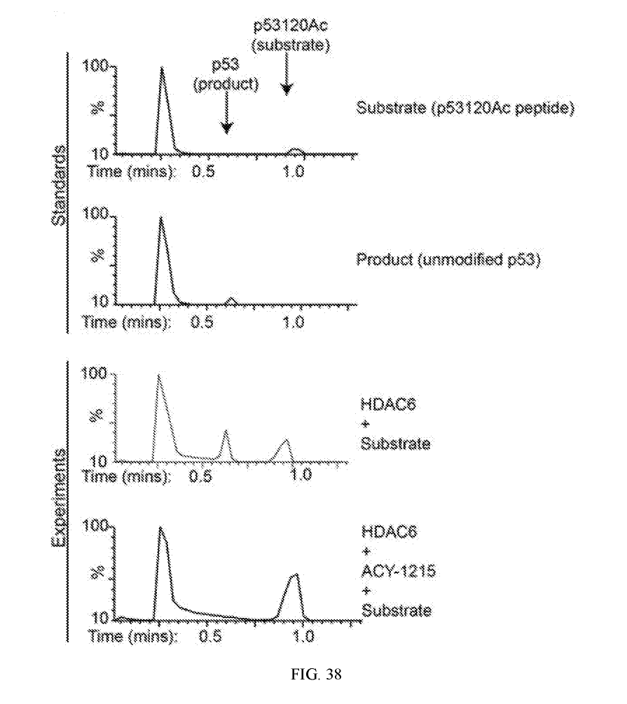

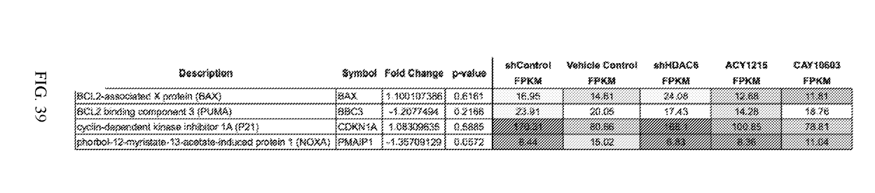

Next, it was determined whether HDAC6 directly catalyses deacetylation of p53K120Ac. An in vitro deacetylation biochemical assay using purified HDAC6 protein and a synthesized p53 peptide containing the K120Ac modification Ac-Leu-His-Ser-Gly-Thr-Ala-Lys(Ac)-Ser-Val-Thr was used as a substrate. Indeed, p53K120Ac modification was efficiently removed by the purified HDAC6. This occurred in a HDAC6 activity-dependent manner because addition of the HDAC6 inhibitor rocilinostat prevented HDAC6 from removing the p53K120Ac modification (FIG. 38). The specific activity of HDAC6 with this substrate was 3.4±0.4 nmol product·nmol enzyme⁻¹·min⁻¹, which was comparable to that of 10.5±0.5 nmol product·nmol enzyme⁻¹·min⁻¹ measured for the standard assay substrate Ala-Lys(Ac)-Ala-NH₂. Moreover, addition of the HDAC6 inhibitor ACY1215 blocked this activity (FIG. 38). It was concluded that p53K120Ac is a direct substrate of HDAC6's deacetylase activity.

### Example 6

### HDAC6 Inhibition Promotes p53-Transcription-Independent Apoptosis

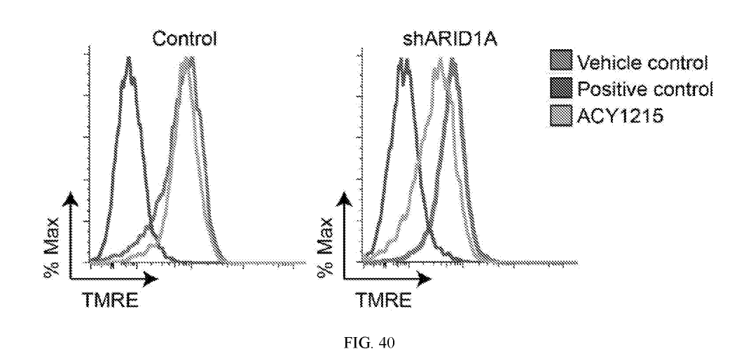

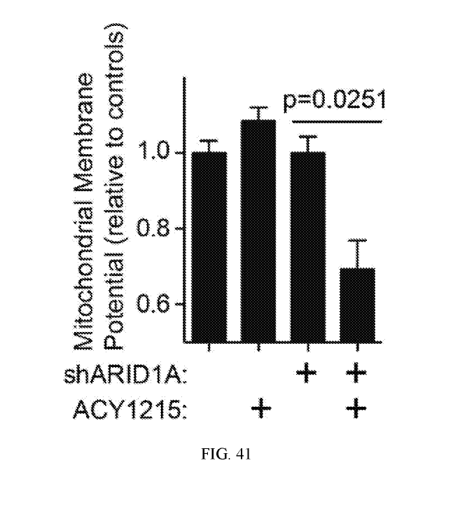

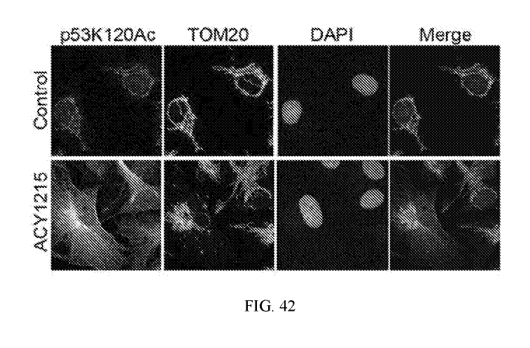

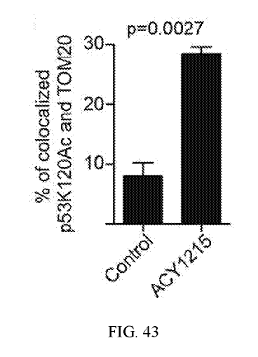

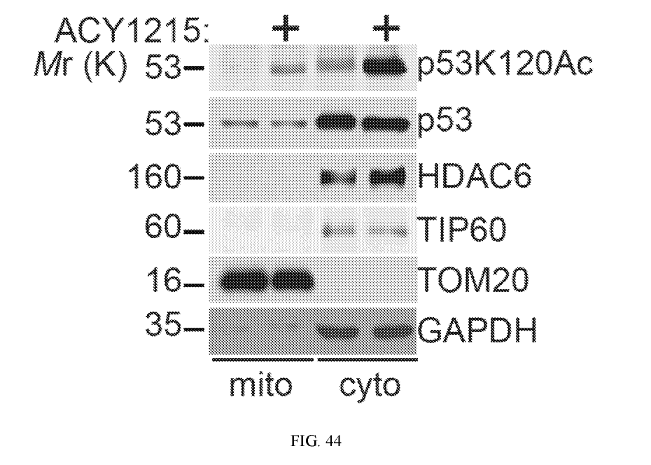

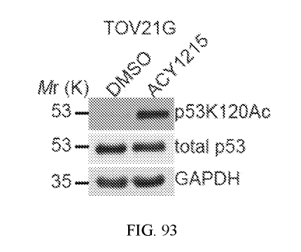

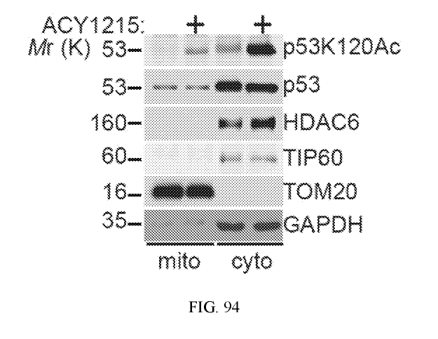

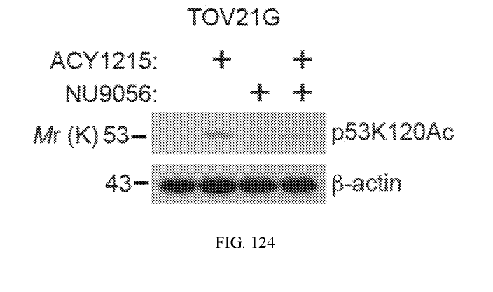

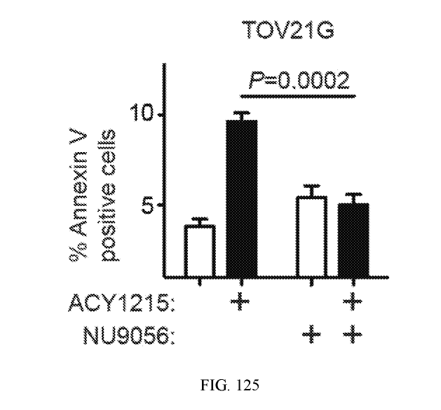

Apoptosis induced by HDAC6 inhibition in ARID1A inactivated cells is p53 dependent and is correlated with upregulation of p53K120Ac (FIG. 28 to FIG. 31, FIG. 35 to FIG. 38, FIG. 89 to FIG. 93). Notably, p53K120Ac promotes apoptosis in both a transcription-dependent manner through upregulating p53 target genes such as BAX and PUMA and transcription-independent mechanism through its cytoplasmic localization into mitochondria. Mellert and McMahon, Trends Biochem. Sci. 2009, 34, 571-578; Sykes et al. Mol. Cell 2006, 24, 841-851; Sykes et al. J. Biol. Chem. 2009, 284, 20197-20205; Tang, Y. et al. Mol. Cell 2006, 24, 827-839. Thus, transcriptional changes by RNA-seq after HDAC6 inhibition were first evaluated using two different HDAC6 inhibitors (namely rocilinostat or CAY10603) or HDAC6 knockdown. Gene expression profiling did not reveal a canonical p53-dependent apoptotic pathway by HDAC6 inhibition (GEO Accession Number: GSE84405). For example, known p53K120Ac target genes such as BAX and PUMA were not significantly upregulated by HDAC6 inhibition (FIG. 39, FIG. 40, and FIG. 41). This indicates that p53 may regulate apoptosis induced by HDAC6 inhibition in a transcription-independent manner. p53K120Ac can also promote apoptosis through its mitochondrial localization (Sykes et al. J. Biol. Chem. 2009, 284, 20197-20205). The localization of p53K120Ac to the mitochondria was measured following HDAC6 inhibition in ARID1A-mutated cells. Immunofluorescence analysis revealed that HDAC6 inhibition induced a significant increase in co-localization of p53K120Ac and the mitochondrial marker TOM20 (FIG. 42, FIG. 43, FIG. 124, and FIG. 125). Notably, NU9056, an inhibitor of TIP60 that acetylates p53K120 31, suppressed apoptosis induced by ACY1215, which correlated with the reduction of the p53K120Ac levels. Cellular fractionation showed an increase in p53K120Ac in the mitochondrial fraction in HDAC6 inhibitor rocilinostat treated cells compared to controls (FIG. 44).

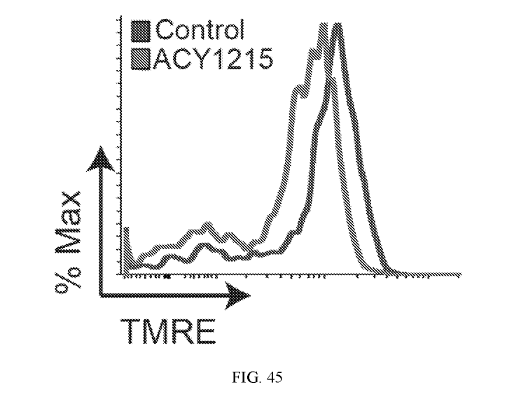

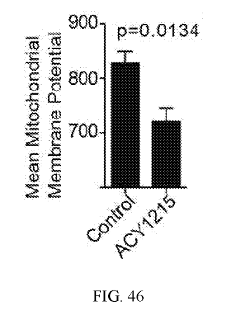

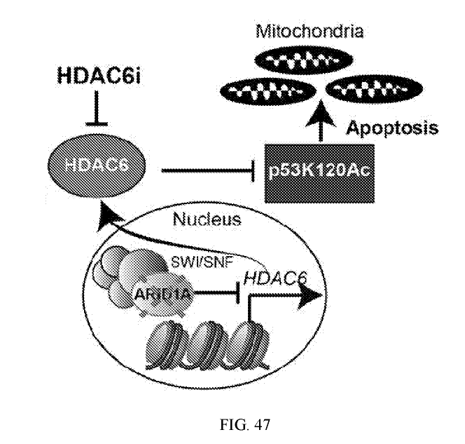

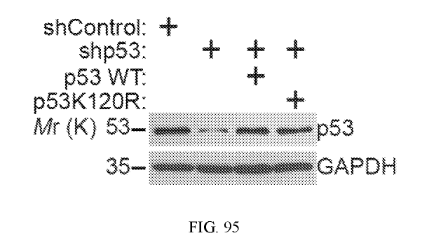

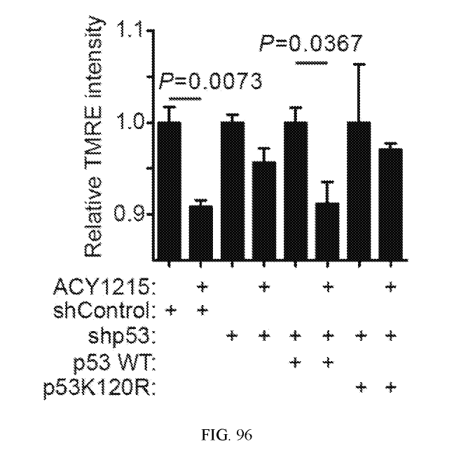

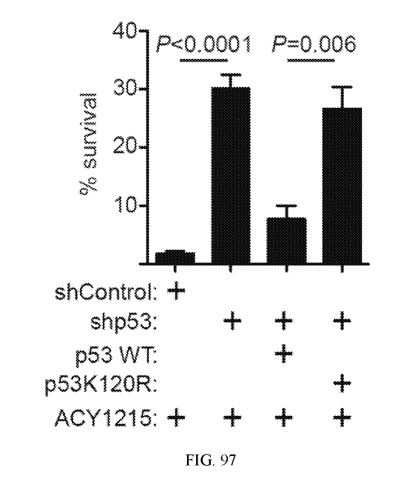

Mitochondrial p53K120Ac promotes apoptosis through decreasing mitochondrial membrane potential. Chen et al. Mol. Cancer Res. 2011, 9, 448-461. Indeed, rocilinostat significantly decreased the mitochondrial membrane potential in ARID1A-mutated cells (FIG. 45 and FIG. 46). Mitochondrial membrane potential decrease by ACY1215 was both p53 and p53K120Ac dependent, because knockdown of p53 suppressed the observed decrease in mitochondrial membrane potential and this was rescued by wildtype p53 but not a p53K120R mutant (FIG. 95 and FIG. 96). Indeed, wildtype p53 but not the p53K120R mutant rescued the p53 knockdown-mediated impairment of ACY1215-induced growth inhibition (FIG. 97). Consistent with the observed selectivity against ARID1A inactivation by HDAC6 inhibition (FIG. 1 to FIG. 4), ARID1A knockdown in ARID1A-wildtype cells significantly decreased mitochondrial membrane potential in cells treated with rocilinostat compared with controls (FIG. 40 and FIG. 41). This is consistent with the observed upregulation of apoptotic markers such as cleaved-PARP and an increase in Annexin V positive cells in ARID1A knockdown cells following HDAC6 inhibitor treatment (FIG. 22 and FIG. 23). Together, it was concluded that HDAC6 inhibition promotes transcription-independent apoptosis that correlates with p53K120Ac mitochondrial localization (FIG. 47 and FIG. 95).

### Example 7

### HDAC6 Inhibitor Improves the Survival of Mice Bearing ARID1A-Mutated Ovarian Tumors

HDAC6 inhibitors such as rocilinostat are now in clinical trials for other malignancies such as myeloma and lymphoma. Seidel et al. Epigenomics 2015, 7, 103-118. Clinical studies show that the HDAC6 inhibitor rocilinostat is well-tolerated without a dose-limiting toxicity. Santo et al. Blood 2012, 119, 2579-2589. To determine the effects of HDAC6 inhibition in vivo on the growth of ARID1A-mutated tumors, luciferase-expressing ARID1A-mutated TOV21G cells were orthotopically transplanted into the bursa-sac covering the ovary of immunocompromised nude mice to mimic the tumor microenvironment. The injected ARID1A wild-type or mutant cells were allowed to grow for 2 weeks to establish the orthotopic tumors. Mice were then randomized and treated daily with vehicle control or rocilinostat (50 mg/kg) by intraperitoneal (i.p.) injection, the same dose as previously reported in Putcha et al. Breast Cancer Res. 2015, 17, 149.

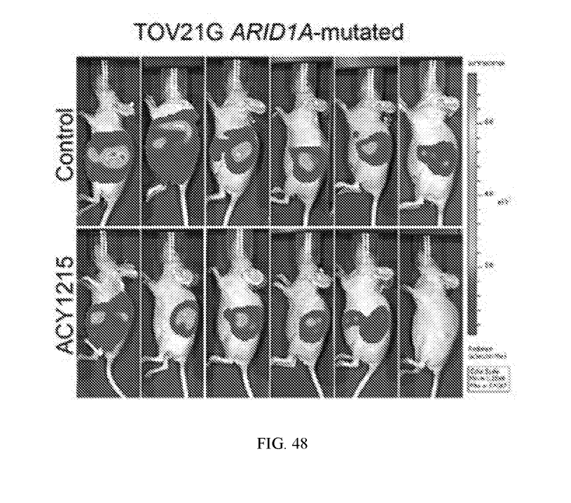

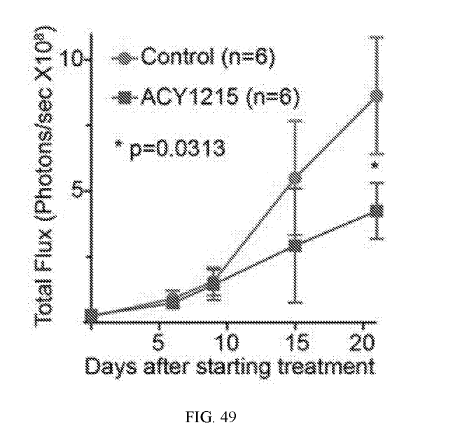

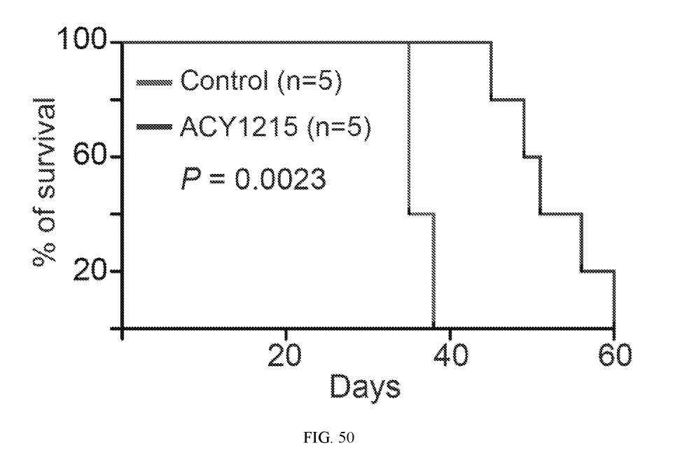

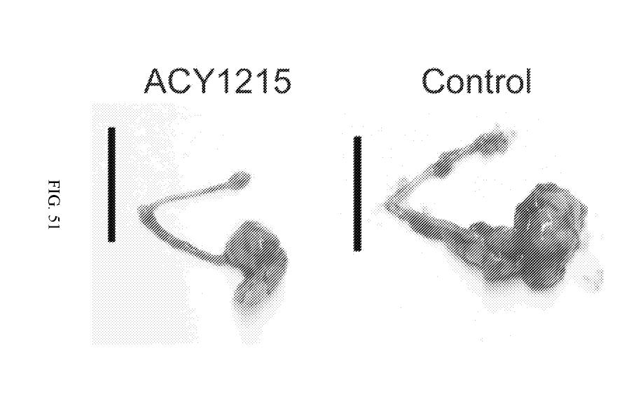

Indeed, rocilinostat treatment significantly inhibited the growth of ARID1A-mutated tumors (FIG. 48 and FIG. 49). The survival of the treated mice after stopping the treatment regimens was followed. Rocilinostat significantly improved the survival of mice bearing the orthotopically-transplanted ARID1A-mutated tumors compared with controls (FIG. 50). Specifically, the median survival was improved from 35 days in the vehicle control group to 51 days in the rocilinostat treated group (p=0.0023). Thus, it was concluded that the HDAC6 inhibitor rocilinostat significantly improves the survival of mice bearing ARID1A-mutated tumors.

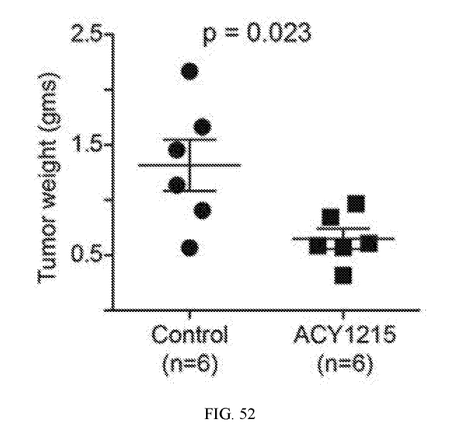

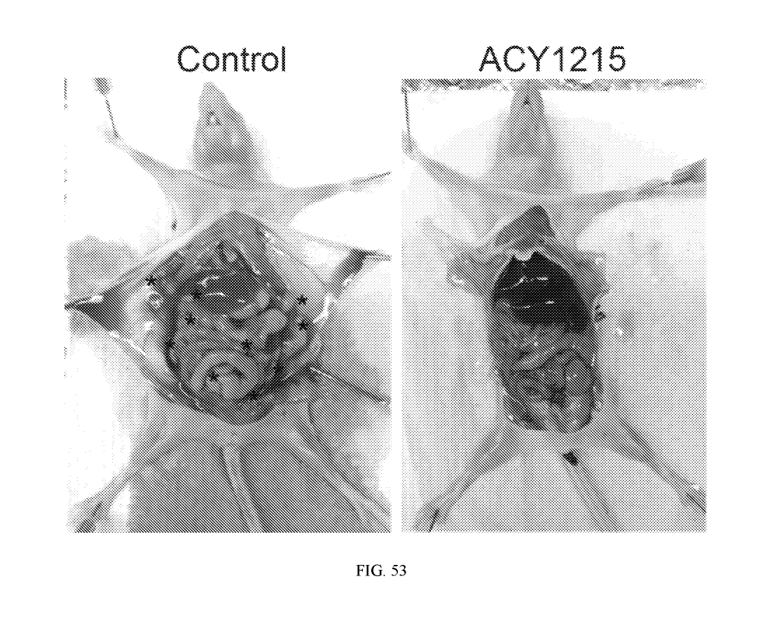

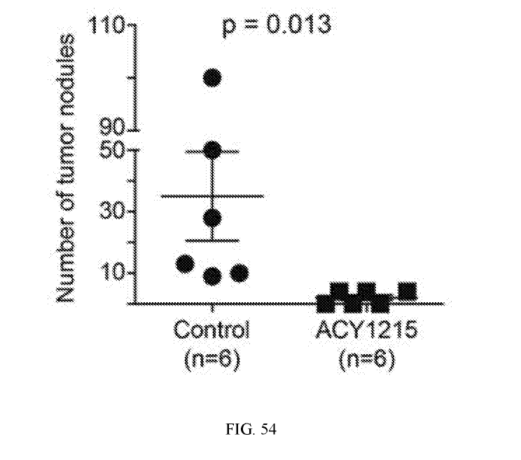

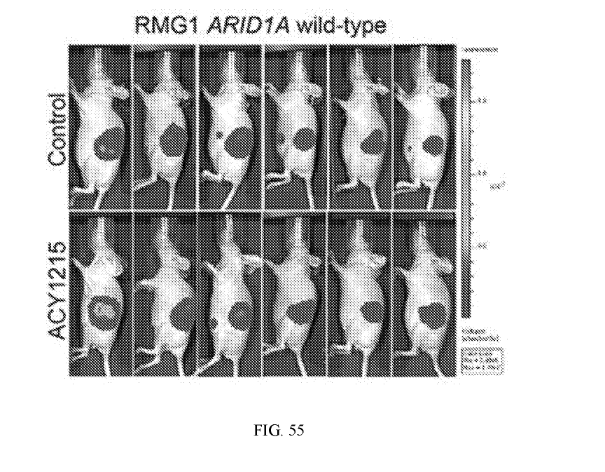

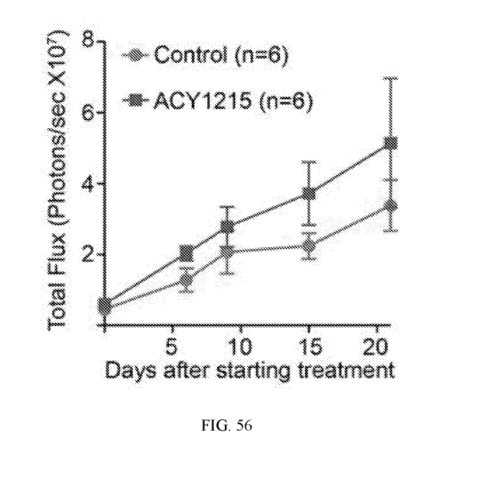

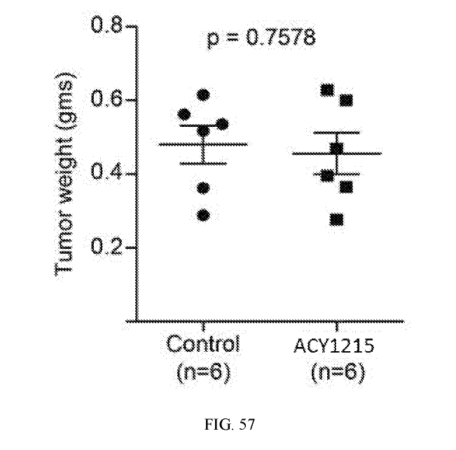

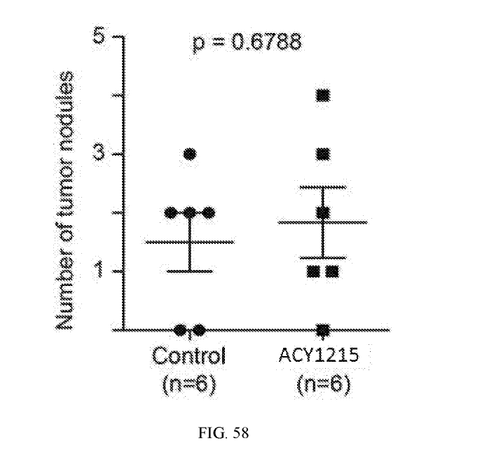

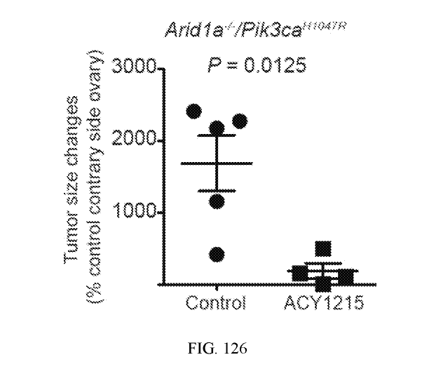

The effects of HDAC6 inhibitor rocilinostat on tumor burden of the transplanted ARID1A-mutated or wild types cells were next directly examined. Indeed, using tumor weight as a surrogate for tumor burden, it was found that rocilinostat treatment significantly reduced the burden of ARID1A-mutated tumors (FIG. 51 and FIG. 52). Likewise, ACY1215 significantly suppressed the tumour growth in the conditional Arid1a−/−/Pik3caH1047R genetic clear cell ovarian tumour mouse model 6 (FIG. 126). Ovarian cancer often progresses by disseminating to the intraperitoneal cavity. Cho and Shih, Annu. Rev. Pathol. 2009, 4, 287-313. Thus, the number of tumor nodules in peritoneal cavity was quantified following treatment with vehicle control or rocilinostat in the pre-established ARID1A-mutated tumors. There was a significant decrease in the number of tumor nodules in rocilinostat treated mice bearing ARID1A-mutated tumors compared to controls (FIG. 53 and FIG. 54). As a control, luciferase-expressing ARID1A wild-type RMG1 cells were orthotopically transplanted in parallel. In contrast to what it was observed in ARID1A mutated tumors, rocilinostat treatment did not significantly affect the growth, tumor burden or dissemination of ARID1A wild-type tumors (FIG. 55, FIG. 56, FIG. 57, and FIG. 58).

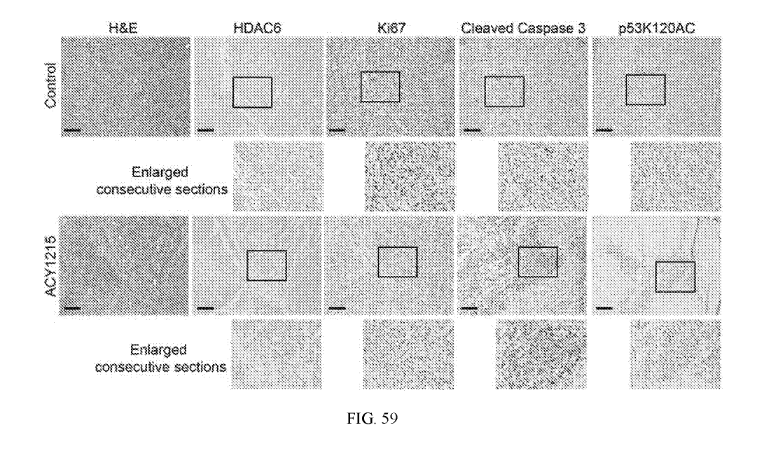

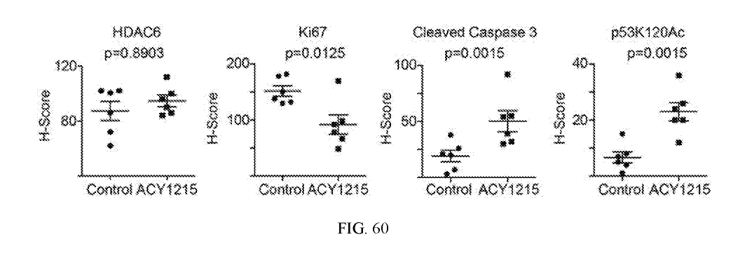

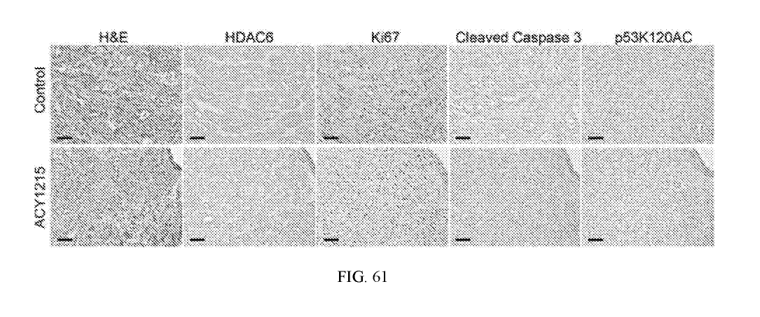

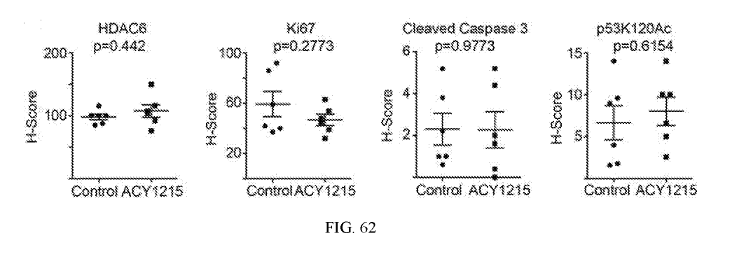

Finally, it was sought to correlate the observed improvement of survival, suppression of tumor growth and reduction in tumor burden in vivo with the molecular pathways revealed for the observed dependence of ARID1A-mutated cells on HDAC6 activity in vitro. To do so, IHC analysis was performed for markers of cell proliferation (Ki67), apoptosis (cleaved caspase 3), HDAC6 and p53K120Ac in dissected ARID1A-mutated tumors treated with rocilinostat or controls (FIG. 59). Rocilinostat significantly decreased the cell proliferation marker Ki67 and increased the apoptotic marker cleaved caspase 3 (FIG. 60). As a control, HDAC6 expression was not affected by rocilinostat. Furthermore, p53K120Ac staining was significantly increased by rocilinostat treatment (FIG. 59 and FIG. 60). As a control, HDAC6 expression was not affected by ACY1215 (FIG. 59 and FIG. 60). Furthermore, p53K120Ac staining was significantly increased by ACY1215 treatment. In contrast, rocilinostat did not affect the expression of Ki67, cleaved caspase 3, or p53K120Ac in ARID1A wild-type tumors (FIG. 61 and FIG. 62). This is consistent with the finding that rocilinostat did not affect the growth of ARID1A wild-type tumors in vivo (FIG. 55, FIG. 56, and FIG. 57). Together, it was concluded that the HDAC6 inhibitor rocilinostat selectively suppresses the growth and dissemination of ARID1A-mutated ovarian tumors and improves the survival of ARID1A-mutated tumor bearing mice. This correlates with a decrease in cell proliferation, an increase in apoptosis and an accumulation of apoptosis-promoting p53K120Ac in the treated ARID1A-mutated tumors.

### Example 8

### HDAC6 Inhibitor Improves the Survival of Mice Bearing ARID1A-Mutated Ovarian Tumors

The data presented herein demonstrated a dependence on HDAC6 activity in ARID1A-mutated cells. This was due to the direct suppression of HDAC6 transcription by ARID1A. Consequently, ARID1A inactivation upregulates HDAC6 expression. Although the SWI/SNF complex mostly promotes the transcription of its target genes, it can also repress gene transcription. Wilson and Roberts, Nat. Rev. Cancer 2011, 11, 481-92. Previous reports established that ARID1A inactivation correlates with silencing of tumor suppressive genes such as PIK3IP1 and CDKN1A. Bitler et al. Nat. Med. 2015, 21, 231-238; Guan et al. Cancer Research 2011, 71, 6718-6727. Here it was showed that HDAC6 is a direct target of ARID1A-mediated transcriptional repression. ARID1A inactivation leads to upregulation of HDAC6; therefore, HDAC6 inhibition is selective against ARID1A inactivation. It has previously been reported in ARID1A knockout mouse models that ARID1A loss promotes the expression of tumor-promoting pro-inflammatory cytokines such as IL6 expression. Chandler et al. Nature Commun. 2015, 6, 6118. This suggests that both transcriptional repression of oncogenic genes and transcriptional activation of tumor suppressor genes contribute to the tumor suppressive activity of ARID1A.

### Example 9

### ARID1A Loss Functionally Inactivates the p53 Tumor Suppressor Pathway

Here it was shown that ARID1A1 inactivation upregulates HDAC6, and HDAC6 directly removes the apoptosis-promoting p53K120Ac post-translational modification. Notably, the biochemical experiments show that p53K120Ac is a novel substrate of HDAC6 and thus identifies a deacetylase for p53 post-translational modification. This suggests that ARID1A mutation functionally inactivates p53 to suppress apoptosis. This is consistent with, and resolves, at least in part, the typical mutual exclusivity of mutations between ARID1A and TP53 in human cancers. Guan et al. Cancer Research 2011, 71, 6718-6727. Previous studies showed that p53K120Ac selectively regulates apoptosis, while it does not affect the expression of cell cycle regulatory p53 target genes such as CDKN1A . Sykes et al. J. Biol. Chem. 2006, 284, 20197-20205. Interestingly, ARID1A collaborates with p53 to regulate CDKN1A expression. Guan et al. Cancer Research 2011, 71, 6718-6727. Thus, ARID1A inactivation contributes to functional inactivation of the p53 tumor suppressor by both indirectly suppressing apoptosis-promoting p53K120Ac through upregulating HDAC6 and directly impairing expression of p53 target cell cycle regulatory genes such as CDKN1A.

In summary, it is demonstrated that targeting HDAC6 activity through the use of HDAC6 inhibitors in ARID1A-mutated cells represents a novel therapeutic strategy. This newly developed therapeutic strategy will be an example of precision medicine because it is based on ARID1A mutational status and the mutually exclusiveness of ARID1A and TP53 mutations. Notably, HDAC6 inhibitors such as rocilinostat are well-tolerated and show minimal toxicity in clinical trials for hematopoietic malignancies. Santo et al. Blood 2012, 119, 2579-2589. Thus, HDAC6 inhibitors may be used in the treatment of ARID1A-mutated ovarian cancers, a disease with no effective therapy. Given that mutation and loss of expression of ARID1A and genetic alterations in other subunits of ATP-dependent chromatin remodeling complexes are observed in ˜20% of all human cancers (Kadoch et al. Nat. Genet. 2013, 45, 592-601), these findings should have far-reaching implications for developing urgently needed therapeutic strategies.

### Example 10

### Combination Treatment with HDAC6 and EZH2 Inhibitors

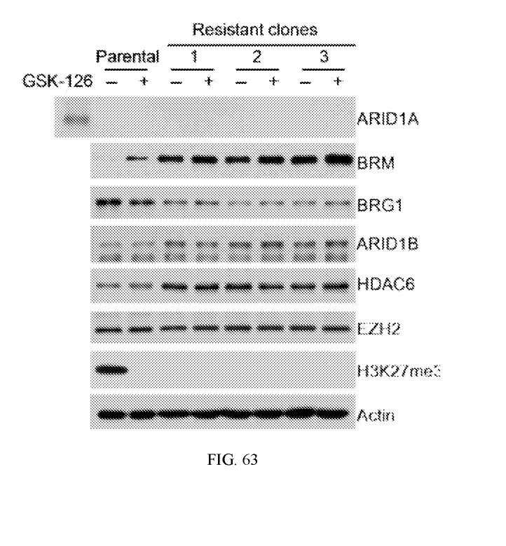

FIG. 63 shows that a switch from BRG1 to BRM catalytic subunit underlies the up-regulation of anti-apoptotic HDAC6 in EZH2 inhibitor resistant cells. Without being bound by theory, this provides a rationale for a combinatorial therapeutic strategy for ARID1A-mutated ovarian cancer by simultaneously inhibiting HDAC6 and EZH2.

1. A method of treating a cancer in a human subject having a mutation in the AT-rich interactive domain-containing protein 1A (ARID1A) gene, comprising the step of administering a therapeutically effective dose of a histone deacetylase 6 (HDAC6) inhibitor to the human subject in need thereof.

2. The method of claim 1, wherein the cancer is selected from the group consisting of ovarian cancer, non-small-cell lung cancer, and renal cancer.

3. The method of claim 2, wherein the cancer is ovarian cancer.

4. The method of claim 3, wherein the ovarian cancer is epithelial ovarian cancer.

5. The method of claim 4, wherein the epithelial ovarian cancer is ovarian clear cell carcinoma.

6. The method of claim 1, further comprising detecting the presence of the mutation in the ARID1A gene in a tissue sample isolated from the human subject.

7. The method of claim 6, wherein the human subject in need thereof has been selected from human subjects suffering from a cancer who do not have a mutation in the ARID1A gene.

8. The method of claim 1, wherein the HDAC6 inhibitor is selected from the group consisting of rocilinostat: 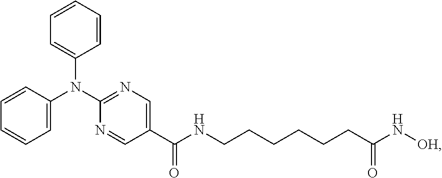 [SMILES: O=C(CCCCCCNC(=O)c1cnc(N(c2ccccc2)c2ccccc2)nc1)NO]
ACY-241: 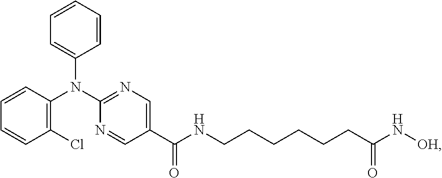 [SMILES: O=C(CCCCCCNC(=O)c1cnc(N(c2ccccc2)c2ccccc2Cl)nc1)NO]
CAY10603: 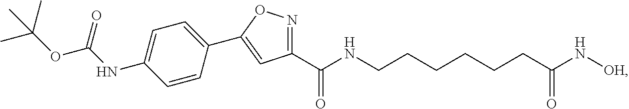 [SMILES: CC(C)(C)OC(=O)Nc1ccc(-c2cc(C(=O)NCCCCCCC(=O)NO)no2)cc1]
Tubastatin A: 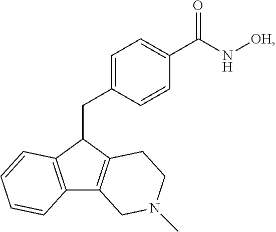 [SMILES: CN1CCC2=C(C1)c1ccccc1C2Cc1ccc(C(=O)NO)cc1]
HPOB: 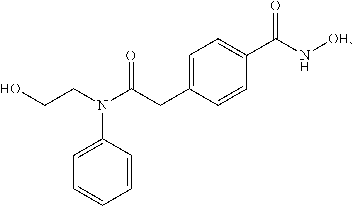 [SMILES: O=C(NO)c1ccc(CC(=O)N(CCO)c2ccccc2)cc1]
tubacin: 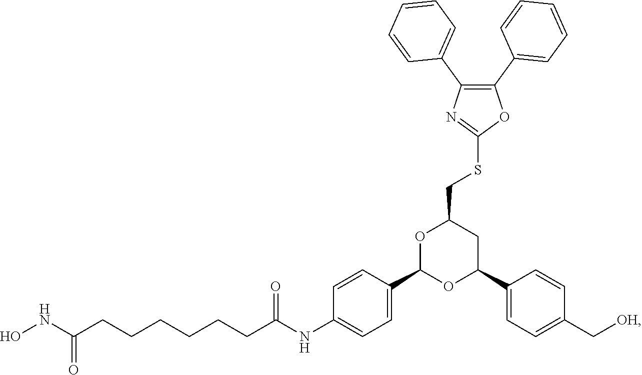 [SMILES: O=C(CCCCCCC(=O)Nc1ccc([C@H]2O[C@@H](CSc3nc(-c4ccccc4)c(-c4ccccc4)o3)C[C@@H](c3ccc(CO)cc3)O2)cc1)NO]
BATCP: 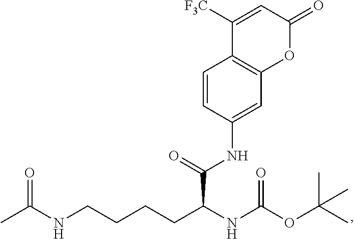 [SMILES: CC(=O)NCCCC[C@H](NC(=O)OC(C)(C)C)C(=O)Nc1ccc2c(C(F)(F)F)cc(=O)oc2c1]
panobinostat: 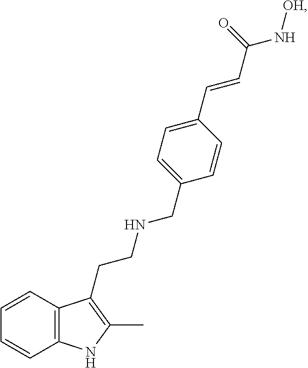 [SMILES: Cc1[nH]c2ccccc2c1CCNCc1ccc(/C=C/C(=O)NO)cc1]
and vorinostat: 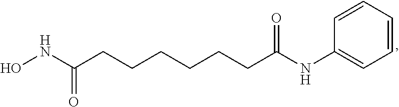 [SMILES: O=C(CCCCCCC(=O)Nc1ccccc1)NO]
and pharmaceutically acceptable salts, solvates, hydrates, cocrystals, or prodrugs thereof.

9. The method of claim 1, further comprising the step of administering a second therapeutically effective dose of an enhancer of zeste homolog 2 (EZH2) inhibitor to the human subject.

10. The method of claim 9, wherein the HDAC6 inhibitor is administered to the subject concurrently with the administration of the EZH2 inhibitor.

11. The method of claim 9, wherein the HDAC6 inhibitor is administered to the subject before administration of the EZH2 inhibitor.

12. The method of claim 9, wherein the HDAC6 inhibitor is administered to the mammal after administration of the EZH2 inhibitor.

13. The method of claim 9, wherein the EZH2 inhibitor is selected from the group consisting of (S)-1-(sec-butyl)-N-((4,6-dimethyl-2-oxo-1,2-dihydropyridin-3-yl)methyl)-3-methyl-6-(6-(piperazin-1-yl)pyridin-3-yl)-1H-indole-4-carboxamide (GSK126): 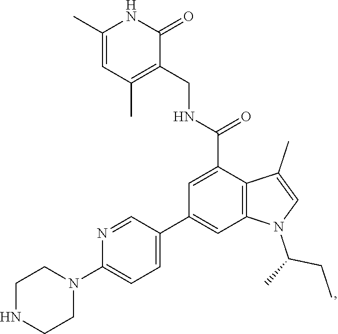 [SMILES: CC[C@H](C)n1cc(C)c2c(C(=O)NCc3c(C)cc(C)[nH]c3=O)cc(-c3ccc(N4CCNCC4)nc3)cc21]
tazemetostat: 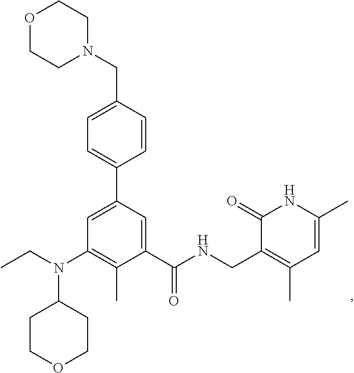 [SMILES: CCN(c1cc(-c2ccc(CN3CCOCC3)cc2)cc(C(=O)NCc2c(C)cc(C)[nH]c2=O)c1C)C1CCOCC1]
(R,Z)-1-(1-(1-(ethylsulfonyl)piperidin-4-yl)ethyl)-N-((2-hydroxy-4-methoxy-6-methylpyridin-3-yl)methyl)-2-methyl-1H-indole-3-carbimidic acid (CPI-169): 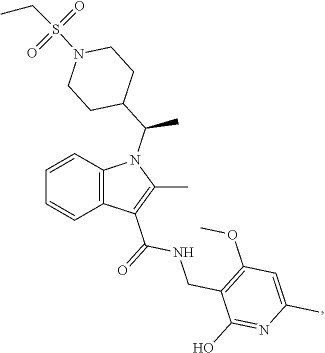 [SMILES: CCS(=O)(=O)N1CCC([C@@H](C)n2c(C)c(C(=O)NCc3c(OC)cc(C)nc3O)c3ccccc32)CC1]
1-cyclopentyl-N-((4,6-dimethyl-2-oxo-1,2-dihydropyridin-3-yl)methyl)-6-(4-(morpholinomethyl)phenyl)-1H-indazole-4-carboxamide (EPZ-5687): 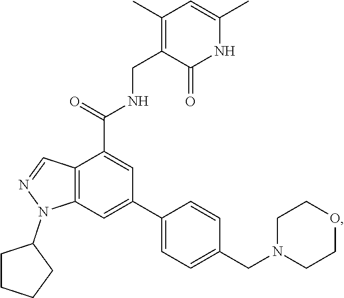 [SMILES: Cc1cc(C)c(CNC(=O)c2cc(-c3ccc(CN4CCOCC4)cc3)cc3c2cnn3C2CCCC2)c(=O)[nH]1]
N-((4,6-dimethyl-2-oxo-1,2-dihydropyridin-3-yl)methyl)-3-(ethyl((1R,4R)-4-((2-methoxyethyl)(methyl)amino)cyclohexyl)amino)-2-methyl-5-(3-morpholinoprop-1-yn-1-yl)benzamide (EPZ-11989): 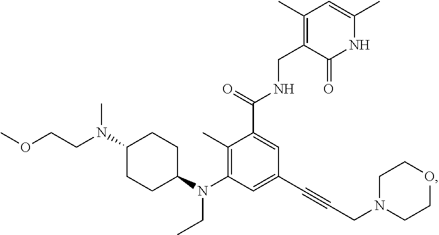 [SMILES: CCN(c1cc(C#CCN2CCOCC2)cc(C(=O)NCc2c(C)cc(C)[nH]c2=O)c1C)[C@H]1CC[C@H](N(C)CCOC)CC1]
1-isopropyl-N-((6-methyl-2-oxo-4-propyl-1,2-dihydropyridin-3-yl)methyl)-6-(2-(4-methylpiperazin-1-yl)pyridin-4-yl)-1H-indazole-4-carboxamide (GSK343): 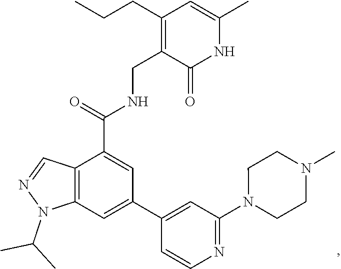 [SMILES: CCCc1cc(C)[nH]c(=O)c1CNC(=O)c1cc(-c2ccnc(N3CCN(C)CC3)c2)cc2c1cnn2C(C)C]
N-((4,6-dimethyl-2-oxo-1,2-dihydropyridin-3-yOmethyl)-1-isopropyl-3-methyl-6-(6-(4-methylpiperazin-1-yl)pyridin-3-yl)-1H-indole-4-carboxamide (GSK503): 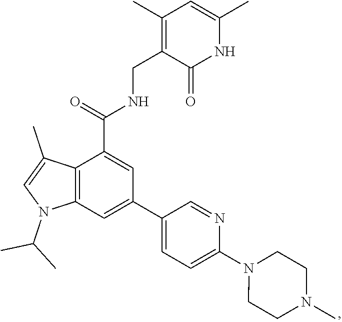 [SMILES: Cc1cc(C)c(CNC(=O)c2cc(-c3ccc(N4CCN(C)CC4)nc3)cc3c2c(C)cn3C(C)C)c(=O)[nH]1]
1-isopropyl-6-(6-(4-isopropylpiperazin-1-yl)pyridin-3-yl)-N-((6-methyl-2-oxo-4-propyl-1,2-dihydropyridin-3-yl)methyl)-1H-indazole-4-carboxamide (UNC-1999): 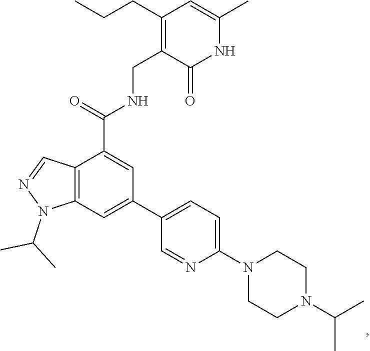 [SMILES: CCCc1cc(C)[nH]c(=O)c1CNC(=O)c1cc(-c2ccc(N3CCN(C(C)C)CC3)nc2)cc2c1cnn2C(C)C]
6-cyano-N-((4,6-dimethyl-2-oxo-1,2-dihydropyridin-3-yl)methyl)-1-(pentan-3-yl)-1H-indole-4-carboxamide (E11): 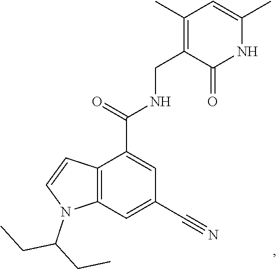 [SMILES: CCC(CC)n1ccc2c(C(=O)NCc3c(C)cc(C)[nH]c3=O)cc(C#N)cc21]
(1S,2R,5R)-5-(4-amino-1H-imidazo[4,5-c]pyridin-1-yl)-3-(hydroxymethyl)-3-cyclopentene-1,2-diol (DZNep): 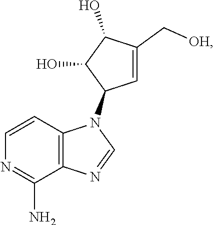 [SMILES: Nc1nccc2c1ncn2[C@@H]1C=C(CO)[C@@H](O)[C@H]1O]
sinefungin: 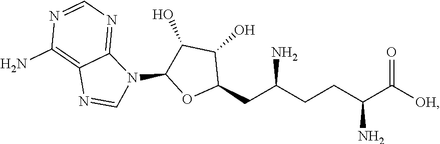 [SMILES: Nc1ncnc2c1ncn2[C@@H]1O[C@H](C[C@@H](N)CC[C@H](N)C(=O)O)[C@@H](O)[C@H]1O]
and pharmaceutically acceptable salts, solvates, hydrates, cocrystals, or prodrugs thereof.
